(19) United States
(12) Patent Application Publication
(10) Pub. No.: US 2024/0116857 A1
(43) Pub. Date: Apr. 11, 2024

(54) QUATERNARY AMMONIUM COMPOUNDS AS ANTIMICROBIALS

(71) Applicants: St. Louis University, St. Louis, MO (US); The Board of Regents of the University of Oklahoma, Norman, OK (US)

(72) Inventors: John K. WALKER, St. Louis, MO (US); Valentin V. RYBENKOV, Norman, OK (US); Ramakumar KINTHADA, St. Louis, MO (US); Hang ZHAO, Norman, OK (US); Elena I. ZGURSKAYA, Norman, OK (US); Terri BOEHM, St. Louis, MO (US); Zoya PETRUSHENKO, Norman, OK (US)

(73) Assignees: Saint Louis University, St. Louis, MO (US); The Board of Regents of the University of Oklahoma, Norman, OK (US)

(21) Appl. No.: 18/348,207

(22) Filed: Jul. 6, 2023

Related U.S. Application Data
(60) Provisional application No. 63/358,808, filed on Jul. 6, 2022.

(51) Int. Cl. C07C 255/51; A61K 45/06; C07C 317/40; C07D 249/04; C07D 295/155; C07D 401/12; C07D 495/04

(52) U.S. Cl. CPC C07C 255/51; A61K 45/06; C07C 317/40; C07D 249/04; C07D 295/155; C07D 401/12; C07D 495/04

(57) ABSTRACT

In one aspect, the present disclosure provides compounds such as those with one or more quaternary ammonium ions joined by one or more aromatic rings. These compounds may have a formula:

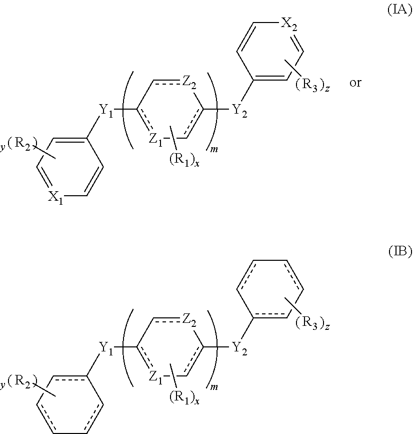

These compounds may be used in the treatment of a microbial infection such as a bacterial infection including infections of drug resistant strains of bacteria.

The present application claims the benefit of priority to U.S. Provisional Application No. 63/358,808, filed on Jul. 6, 2022, the entire contents of which are hereby incorporated by reference.

This invention was made with government support under Grant No. 1R01AI136799-01 awarded by the National Institute of Health. The government has certain rights in the invention.

### BACKGROUND

### 1. Field

This disclosure relates to the fields of biology, biochemistry, chemistry, pharmacology and medicine. In particular, new methods, compounds, and methods of treatment related to microbial infections.

### 2. Related Art

Infectious diseases are currently the second leading cause of death worldwide and the third leading cause of death in economically advanced countries despite the development of antibiotics (Nathan, 2004). Furthermore, the threatening emergence of bacterial resistance to many antibiotics presents a serious challenge for their clinical use. As our antibiotic options continue to shrink, prevention becomes increasingly important in elevating the burden of hospital-associated bacterial infections (Otter et al., 2011; Weber et al., 2010; Barsanti and Woeltje, 2009). Surface decontamination relies heavily on the use of biocides; chemical agents with antimicrobial properties. Among the biocide classes, quaternary amine compounds (QACs) such as benzalkonium chloride (BAC), are frequently used for disinfecting medical equipment, hospital surfaces, patient wounds, and healthcare workers' hands. QACs are recommended for use at concentrations many times their minimal inhibitory concentration (MIC) and effectively eradicate vegetative bacteria at these doses under laboratory conditions. But practical clinical conditions including high bacterial density, the presence of biofilm and organic matter, and high ion concentrations (Klimek and Bailey, 1955; Otter et al., 2015) dramatically decrease the effectiveness of QAC biocides, which allows bacteria to escape death and persist in the hospital environment. Despite their wide use, the antimicrobial effects of QACs are unclear, especially at near inhibitory concentrations. Traditional QACs that have been developed are too toxic for use as pharmaceuticals meaning their only application has been as biocides. Additionally, the incomplete understanding of QAC action has fueled concerns of cross-resistance between biocides and antibiotics. If this were to occur, resistant bacteria could be selected both during antibiotic treatment and clinical disinfection, promoting the propagation of resistant endemic strains. Furthermore, the modification of compounds to include different types of linkers between two or more quaternary ammonium groups is of interest to reduce the toxicity so that these compounds such that they may be useful as pharmaceuticals.

Therefore, there remains a need to develop new compounds for treating bacterial infections.

### SUMMARY

As provided herein, the present disclosure relates to compounds containing one or more quaternary ammonium ions linked by multiple aromatic rings. In some embodiments, the present disclosure provides compounds of the formula:

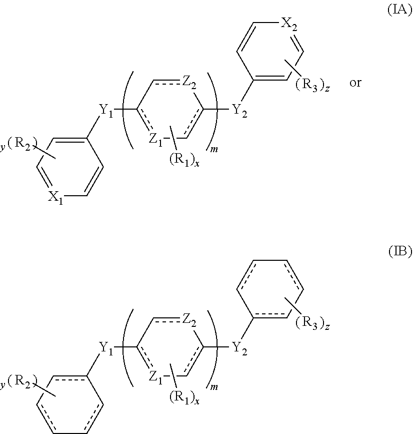

wherein:
m is 1 or 2;
X₁ and X₂ are each C, N, or ⁺NRₐ; wherein:
   Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
   R_{b} is hydrogen, alkyl_{(C≤6)}, or substituted alkyl_{(C≤6)}; and
   a is 0, 1, or 2;
Each R₁ is amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, alkylsulfonyl_{(C≤8)}, substituted alkylsulfonyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x is 0, 1, 2, 3, or 4;
each Z₁ and Z₂ in each ring are each independently C, N, S, or O; R₂ and R₃ are each independently amino, cyano, halo, hydroxy, nitro, aminosulfonyl, hydroxysulfonyl, alkyl_{(C≤12)}, substituted alkyl_{(C≤12)}, alkenyl_{(C≤12)}, substituted alkenyl_{(C≤12)}, alkynyl_{(C≤12)}, substituted alkynyl_{(C≤12)}, heterocycloalkyl_{(C≤8)}, substituted heterocycloalkyl_{(C≤8)}, alkoxy_{(C≤8)}, substituted alkoxy_{(C≤8)}, alkenyloxy_{(C≤8)}, substituted alkenyloxy_{(C≤8)}, alkynyloxy_{(C≤8)}, substituted alkynyloxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; or
a group of the formula: ⁺N(R_{c})₃ wherein:
   each R_{c} are each independently alkyl_{(C≤6)} or substituted alkyl_{(C≤6)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, the compounds are further defined as:

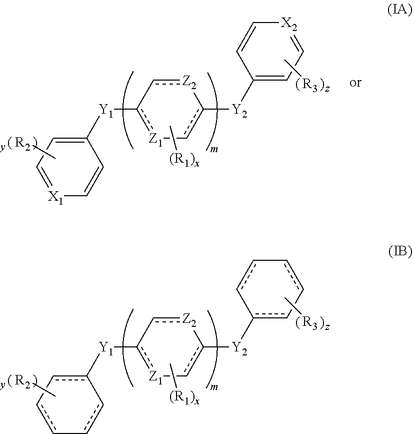

wherein:
m is 1 or 2;
X₁ and X₂ are each C, N, or ⁺NRₐ; wherein:
   Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
   R_{b} is hydrogen, alkyl_{(C≤6)}, or substituted alkyl_{(C≤6)}; and
   a is 0, 1, or 2;
Each R₁ is amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x is 0, 1, 2, 3, or 4;
each Z₁ and Z₂ in each ring are each independently C, N, S, or O; R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; or
a group of the formula: ⁺N(R_{c})₃ wherein:
   each R_{c} are each independently alkyl_{(C≤6)} or substituted alkyl_{(C≤6)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, the compounds are further defined as:

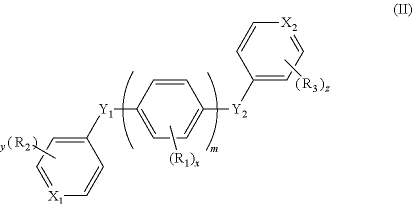

wherein:
m is 1 or 2;
X₁ and X₂ are each CH, N, or NRₐ; wherein:
   Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
   R_{b} is hydrogen, alkyl_{(C≤6)}, or substituted alkyl_{(C≤6)}; and
   a is 0, 1, or 2;
Each R₁ is amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x is 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, the compounds are further defined as:

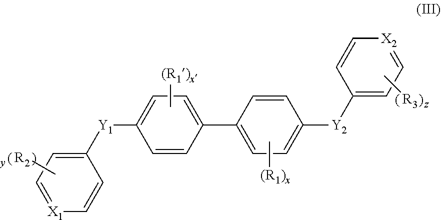

wherein:
X₁ and X₂ are each CH, N, or NRₐ; wherein:
   Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
   R_{b} is hydrogen, alkyl_{(C≤6)}, or substituted alkyl_{(C≤6)}; and
   a is 0, 1, or 2;
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, the compounds are further defined as:

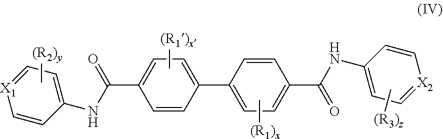

wherein:
X₁ and X₂ are each CH, N, or NRₐ; wherein:
   Rₐ is alkyl_{(C≤6)} or substituted alkyl_{(C≤6)};
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, the compounds are further defined as:

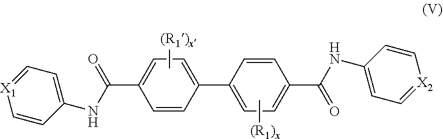

wherein:
X₁ and X₂ are each CH, N, or NRₐ; wherein:
   Rₐ is alkyl_{(C≤6)} or substituted alkyl_{(C≤6)};
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:

—X₃—Y₃—R₄
wherein:
   X₃ is a covalent bond, O, NR_{c}, S, alkanediyl_{(C≤6)}, or substituted alkanediyl_{(C≤6)};
   Y₃ is arenediyl_{(C≤12)}, heteroarenediyl_{(C≤12)}, or a substituted version of either of these groups; and
   R₄ is alkyl_{(C≤12)}, substituted alkyl_{(C≤12)}, or ⁺N(R₅)₃, wherein:
      R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
   or a pharmaceutically acceptable salt thereon.

In some embodiments, m is 1. In other embodiments, m is 2. In some embodiments, Y₁ is C(O)NR_{b}. In some embodiments, R_{b} is hydrogen. In some embodiments, Y₂ is C(O)NR_{b}. In some embodiments, R_{b} is hydrogen. In some embodiments, y is 0, 1, or 2.

In some embodiments, R₂ is alkyl_{(C≤8)} or substituted alkyl_{(C≤8)}. In some embodiments, R₂ is alkyl_{(C≤8)} such as methyl. In other embodiments, R₂ is alkoxy_{(C≤8)} or substituted alkoxy_{(C≤8)}. In some embodiments, R₂ is alkoxy_{(C≤8)} such as methoxy. In other embodiments, R₂ is a group of the formula:

—X₃—Y₃—R₄

wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}.

In some embodiments, X₃ is a covalent bond. In other embodiments, X₃ is O. In some embodiments, X₃ is NR_{c}. In some embodiments, R_{c} is hydrogen. In other embodiments, X₃ is alkanediyl_{(C≤6)} or substituted alkanediyl_{(C≤6)}. In some embodiments, X₃ is alkanediyl_{(C≤6)} such as CH₂. In some embodiments, Y₃ is arenediyl_{(C≤12)} or substituted arenediyl_{(C≤12)}. In some embodiments, Y₃ is arenediyl_{(C≤12)} such as benzenediyl. In other embodiments, Y₃ is heteroarenediyl_{(C≤12)} or substituted heteroarenediyl_{(C≤12)}. In some embodiments, Y₃ is heteroarenediyl_{(C≤12)} such as pyridinediyl. In some embodiments, R₄ is alkyl_{(C≤12)} or substituted alkyl_{(C≤12)}. In some embodiments, R₄ is on a nitrogen atom of a heteroarenediyl group at Y₃. In some embodiments, R₄ is alkyl_{(C≤12)} such as methyl. In other embodiments, R₄ is ⁺N(R₅)₃. In some embodiments, R₅ is alkyl_{(C≤8)} or substituted alkyl_{(C≤8)}. In some embodiments, R₅ is alkyl_{(C≤8)} such as methyl. In some embodiments, z is 0, 1, or 2.

In some embodiments, R₃ is alkyl_{(C≤8)} or substituted alkyl_{(C≤8)}. In some embodiments, R₃ is alkyl_{(C≤8)} such as methyl. In other embodiments, R₃ is alkoxy_{(C≤8)} or substituted alkoxy_{(C≤8)}. In some embodiments, R₃ is alkoxy_{(C≤8)} such as methoxy. In other embodiments, R₃ is a group of the formula:

—X₃—Y₃—R₄

wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}.

In some embodiments, X₃ is a covalent bond. In other embodiments, X₃ is O. In some embodiments, X₃ is NR_{c}. In some embodiments, R_{c} is hydrogen. In other embodiments, X₃ is alkanediyl_{(C≤6)} or substituted alkanediyl_{(C≤6)}. In some embodiments, X₃ is alkanediyl_{(C≤6)} such as CH₂. In some embodiments, Y₃ is arenediyl_{(C≤12)} or substituted arenediyl_{(C≤12)}. In some embodiments, Y₃ is arenediyl_{(C≤12)} such as benzenediyl. In other embodiments, Y₃ is heteroarenediyl_{(C≤12)} or substituted heteroarenediyl_{(C≤12)}. In some embodiments, Y₃ is heteroarenediyl_{(C≤12)} such as pyridinediyl. In some embodiments, R₄ is alkyl_{(C≤12)} or substituted alkyl_{(C≤12)}. In some embodiments, R₄ is on a nitrogen atom of a heteroarenediyl group at Y₃. In some embodiments, R₄ is alkyl_{(C≤12)} such as methyl. In other embodiments, R₄ is ⁺N(R₅)₃. In some embodiments, R₅ is alkyl_{(C≤8)} or substituted alkyl_{(C≤8)}. In some embodiments, R₅ is alkyl_{(C≤8)} such as methyl.

In some embodiments, the compounds are further defined as:

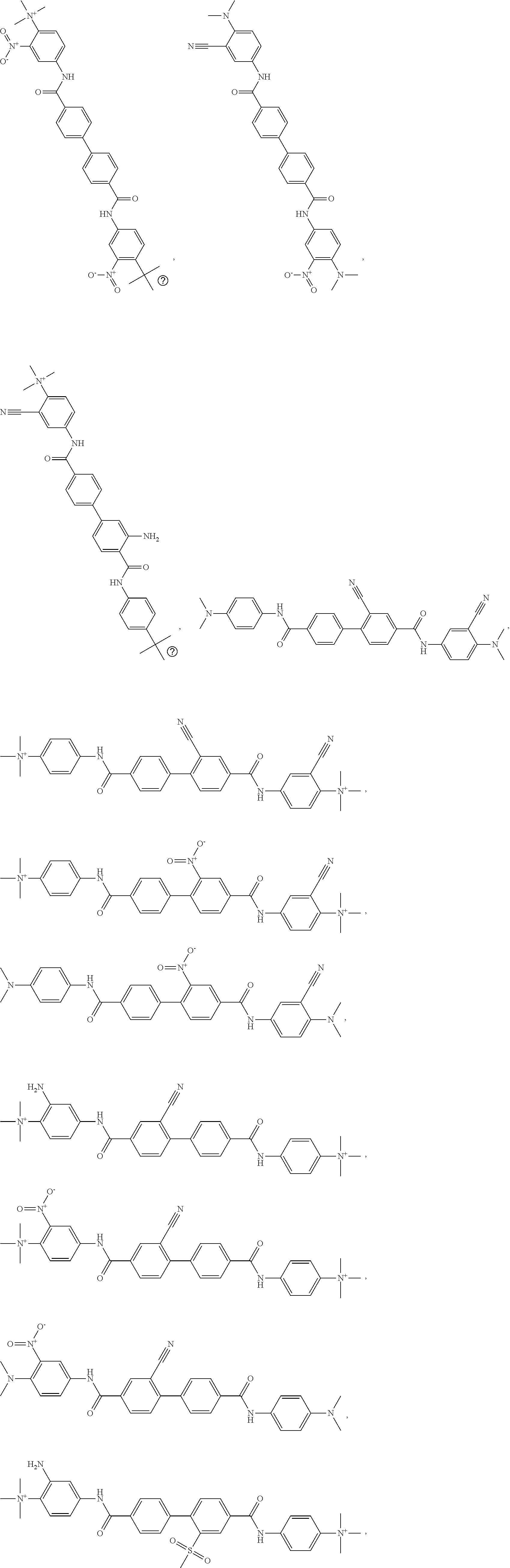

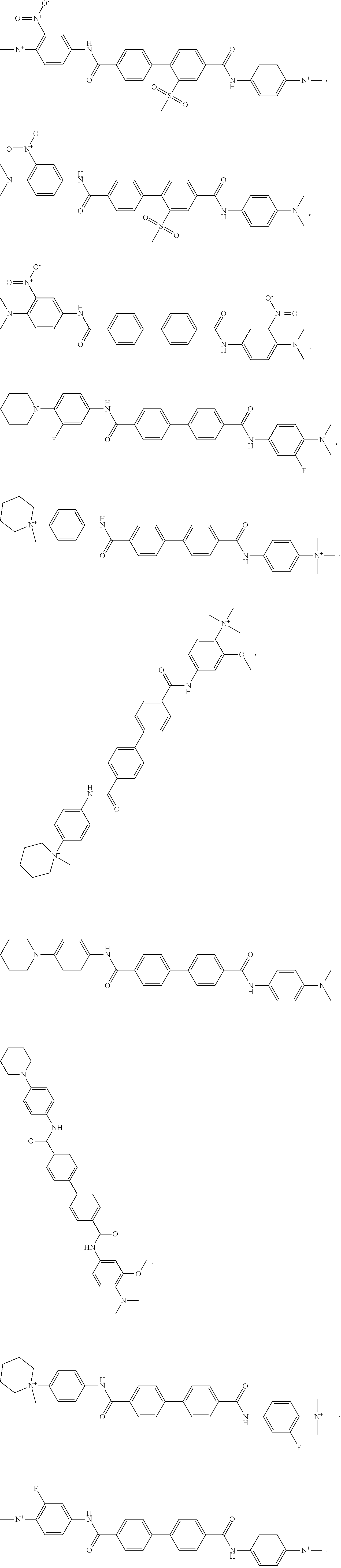

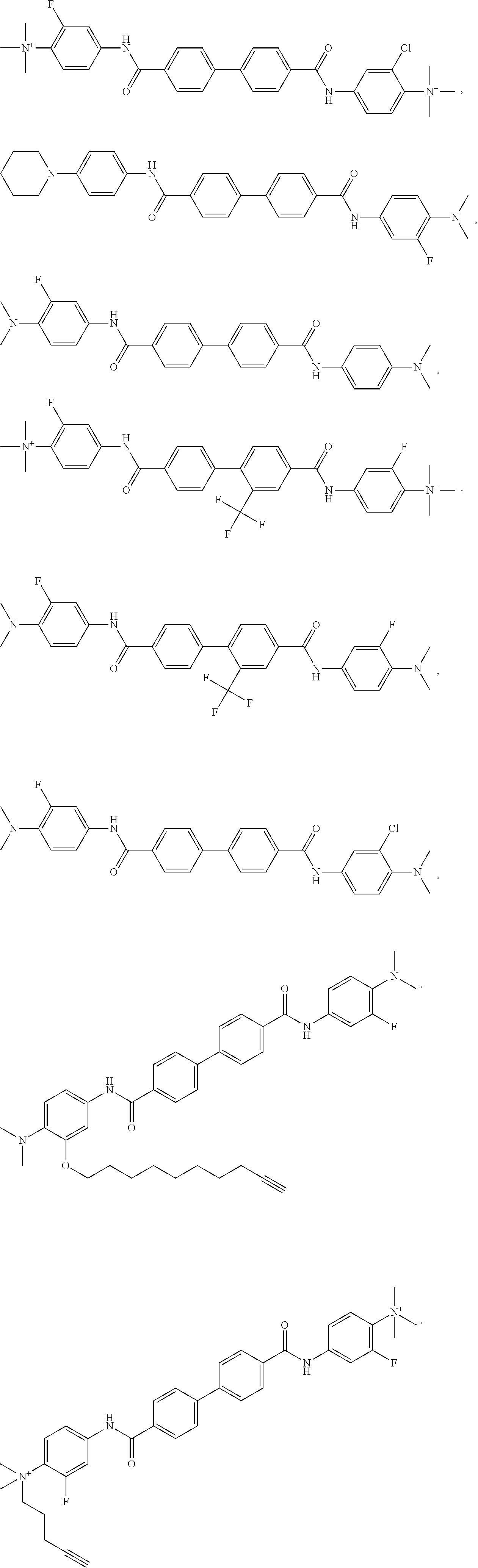

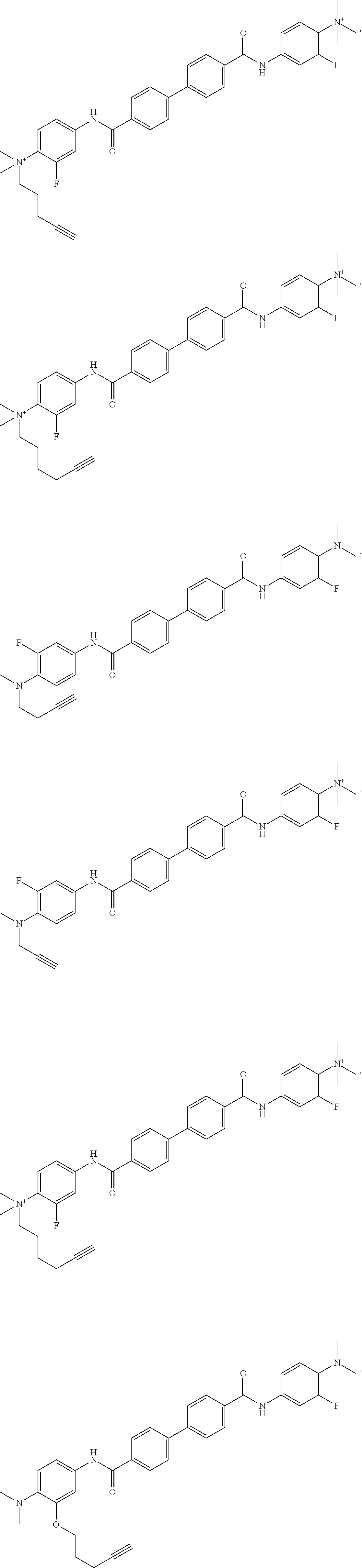

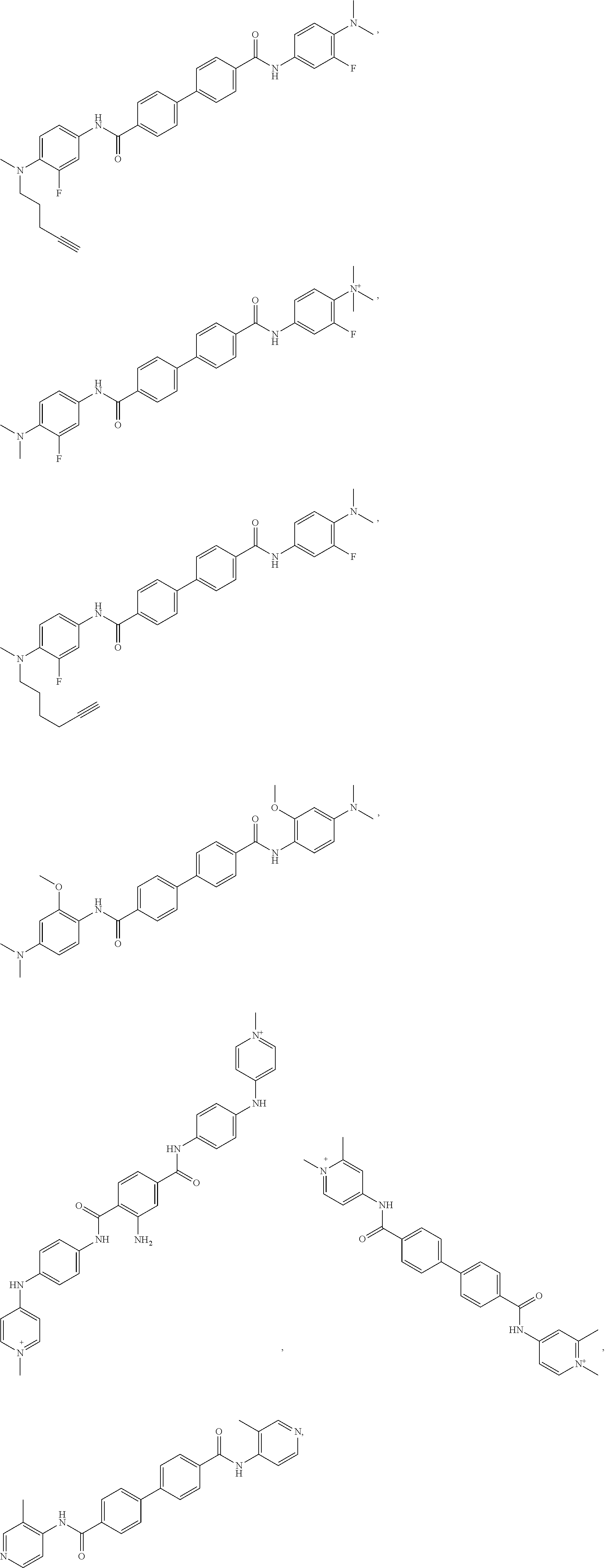

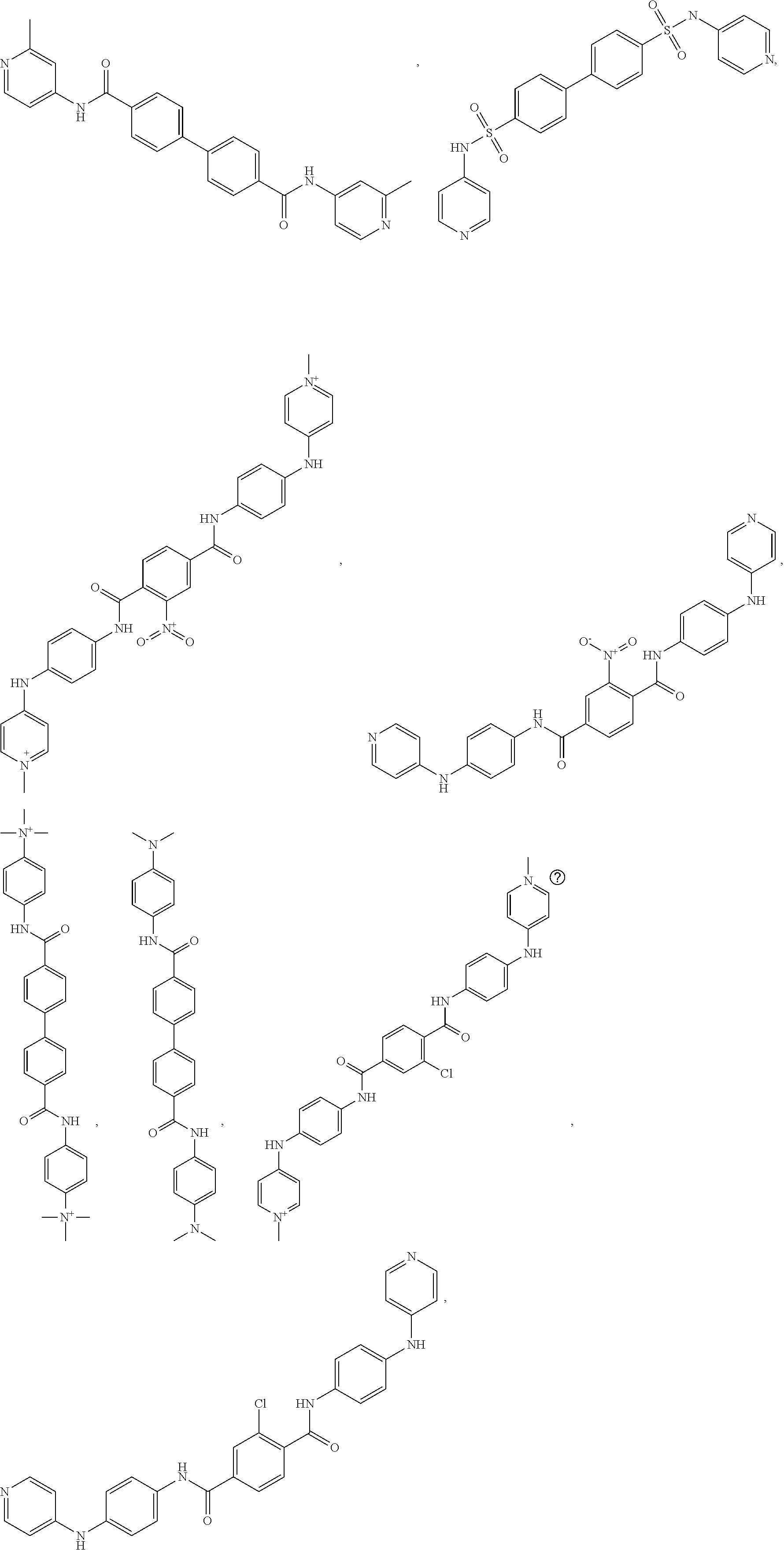

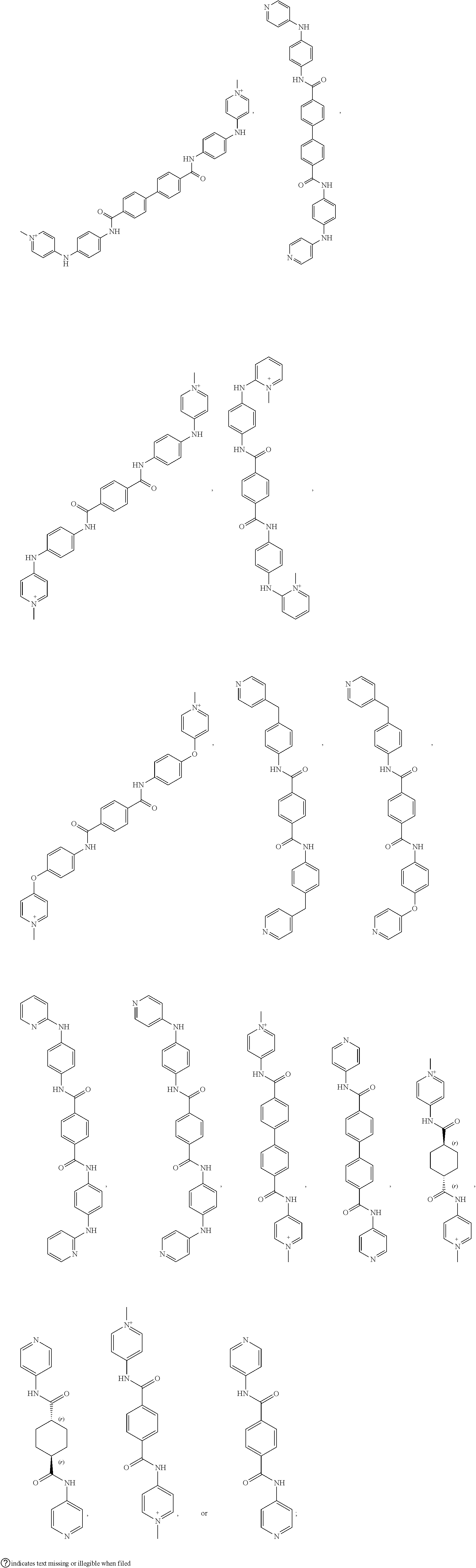

or a pharmaceutically acceptable salt thereof.

In some embodiments, the compounds are further defined as:

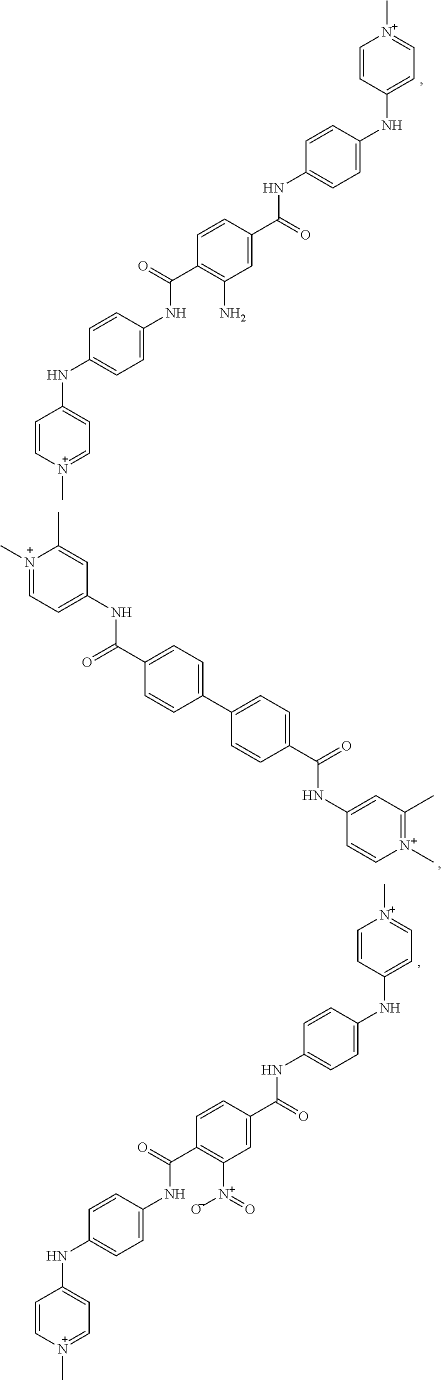

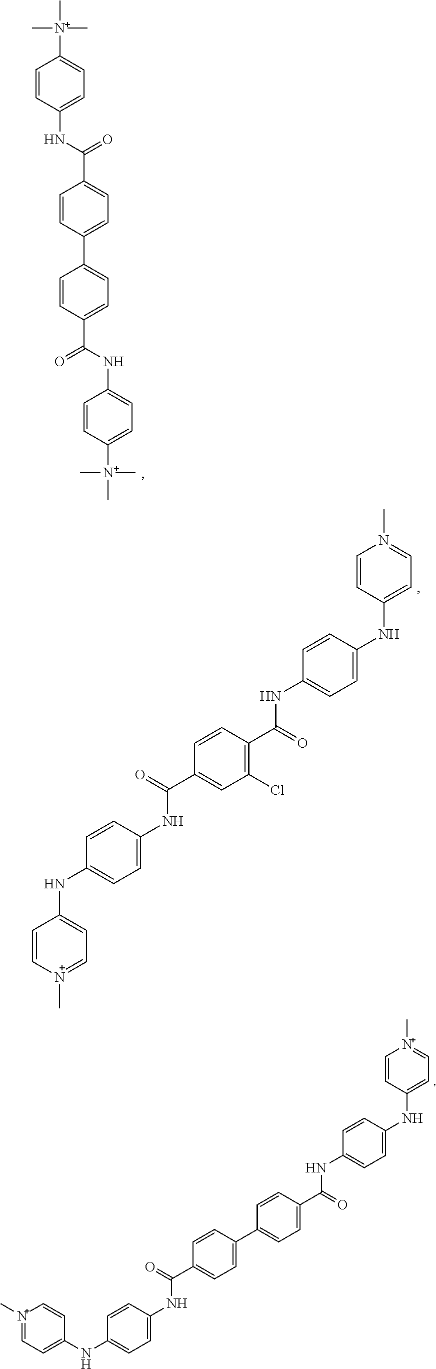

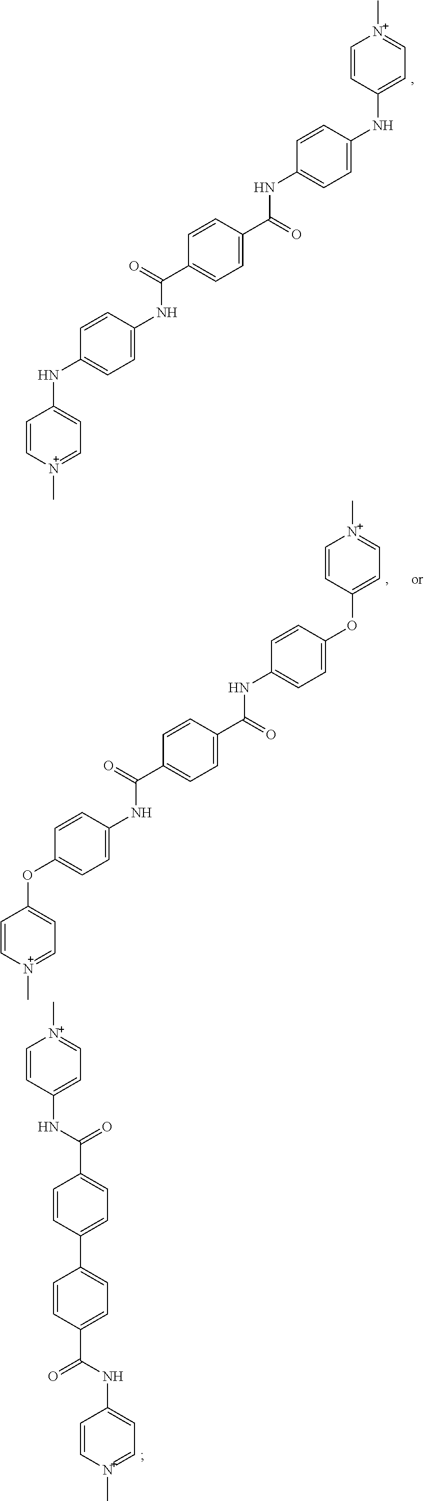 [SMILES: C[n+]1ccc(Nc2ccc(NC(=O)c3ccc(C(=O)Nc4ccc(Nc5cc[n+](C)cc5)cc4)cc3)cc2)cc1 ; C[n+]1ccc(Oc2ccc(NC(=O)c3ccc(C(=O)Nc4ccc(Oc5cc[n+](C)cc5)cc4)cc3)cc2)cc1 ; C[n+]1ccc(NC(=O)c2ccc(-c3ccc(C(=O)Nc4cc[n+](C)cc4)cc3)cc2)cc1]

or a pharmaceutically acceptable salt thereof.

In still another aspect, the present disclosure provides compounds of the formula:

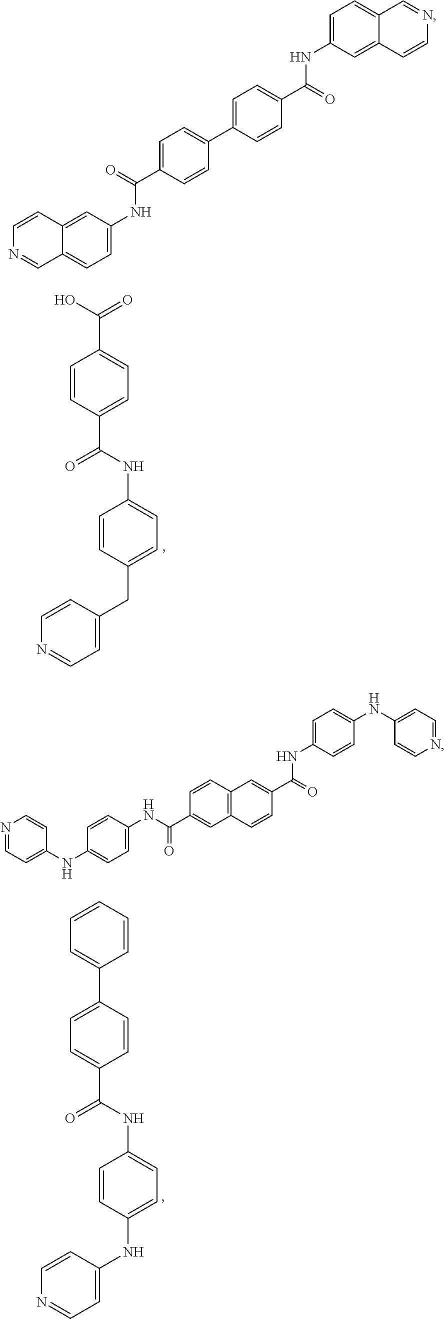

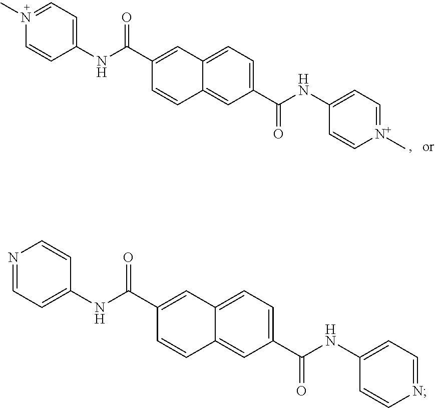

or a pharmaceutically acceptable salt thereof.

In still yet another aspect, the present disclosure provides compounds of the formula:

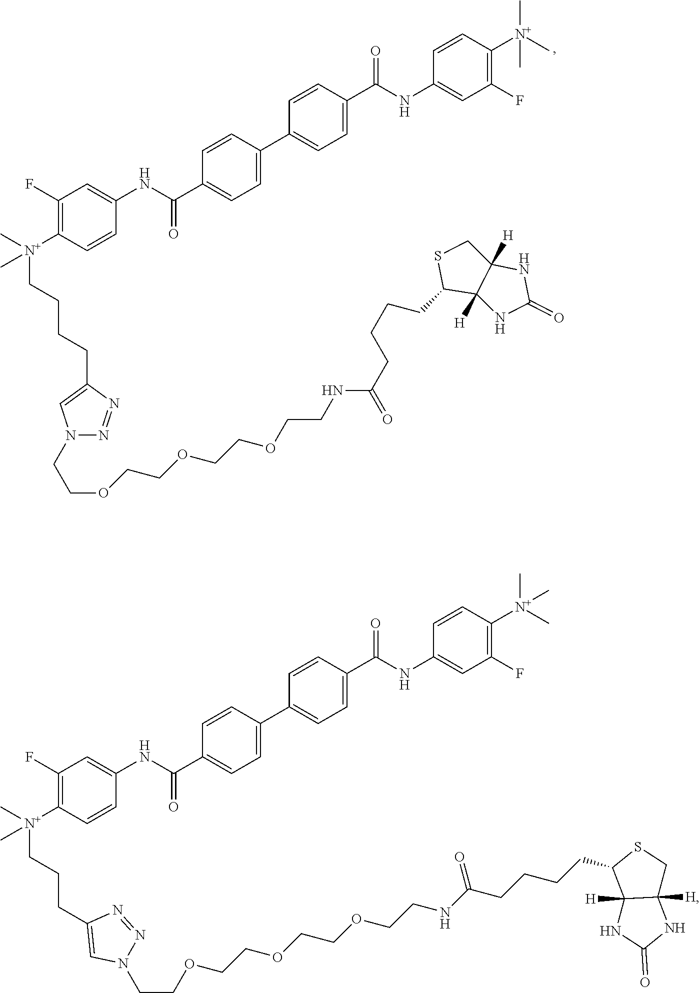

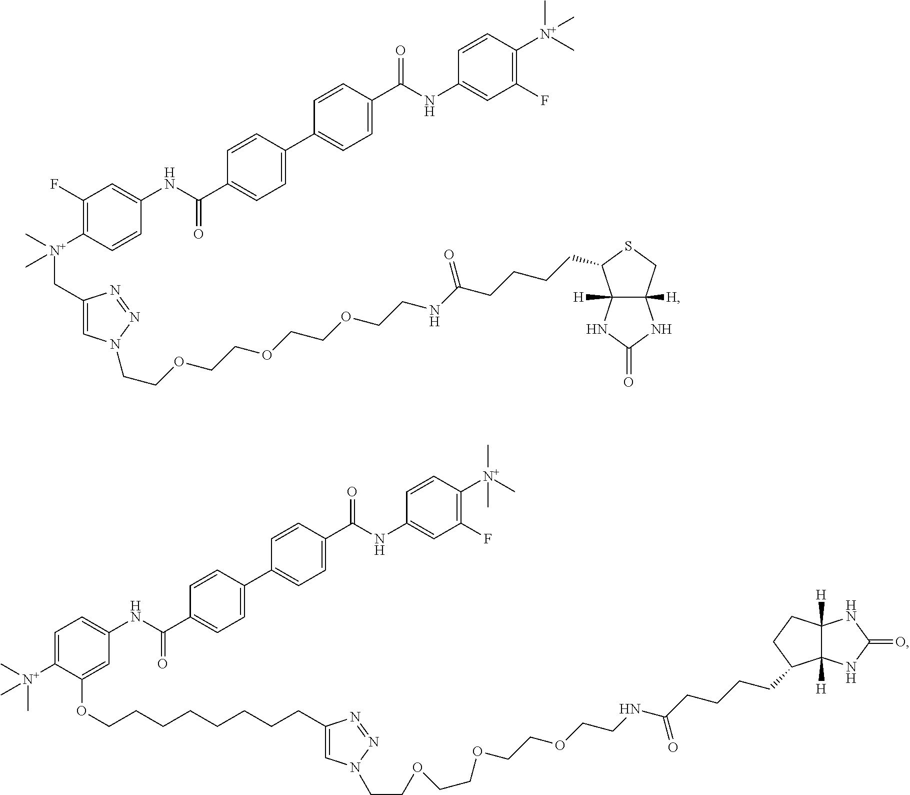

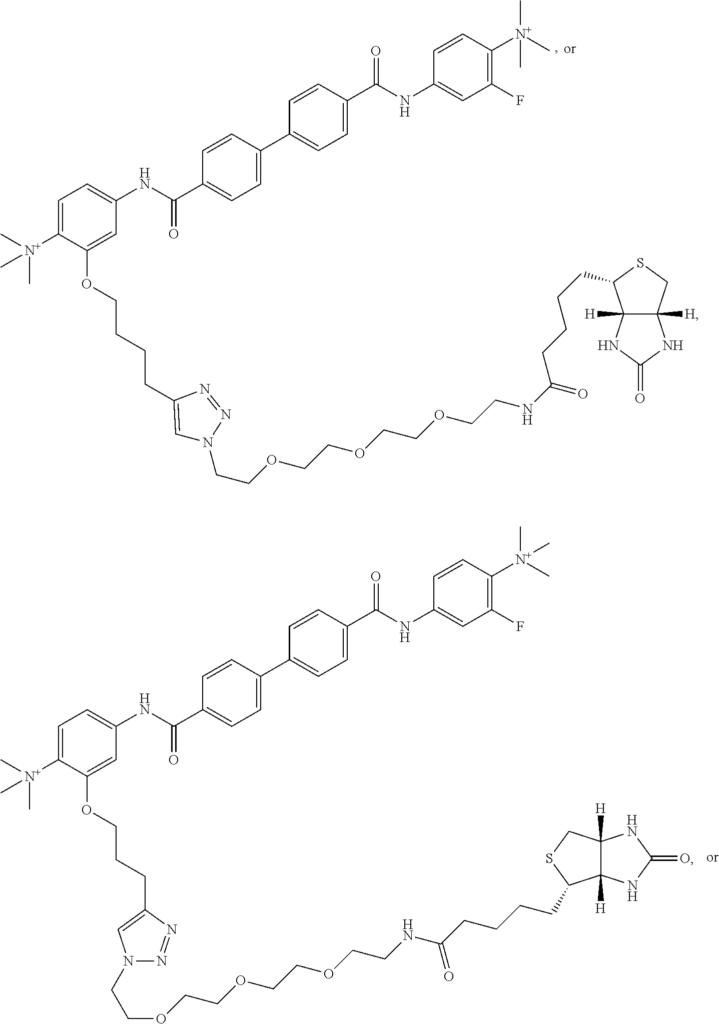

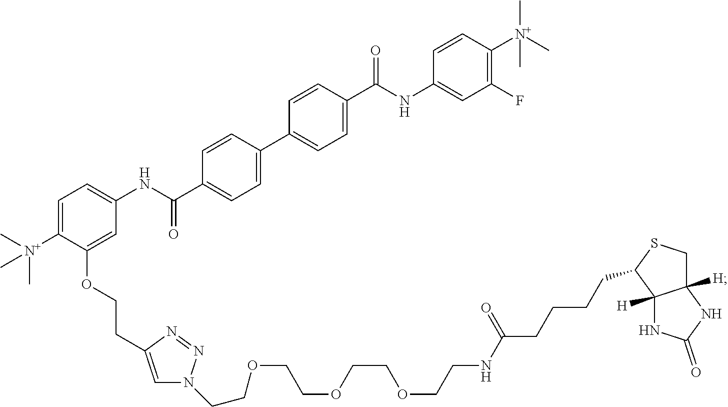

or a pharmaceutically acceptable salt thereof.

In yet another aspect, the present disclosure provides compounds of the formula:

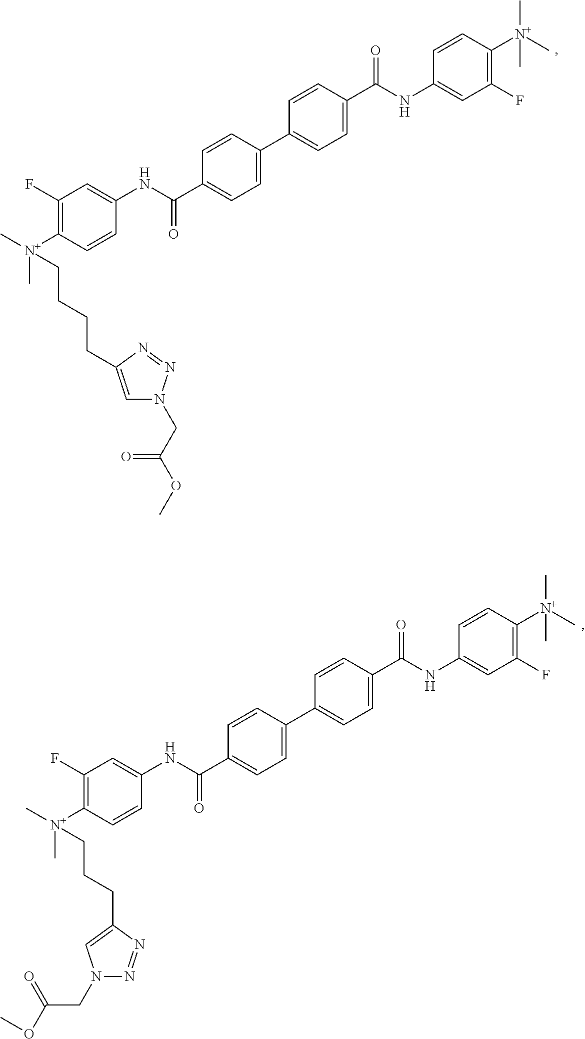

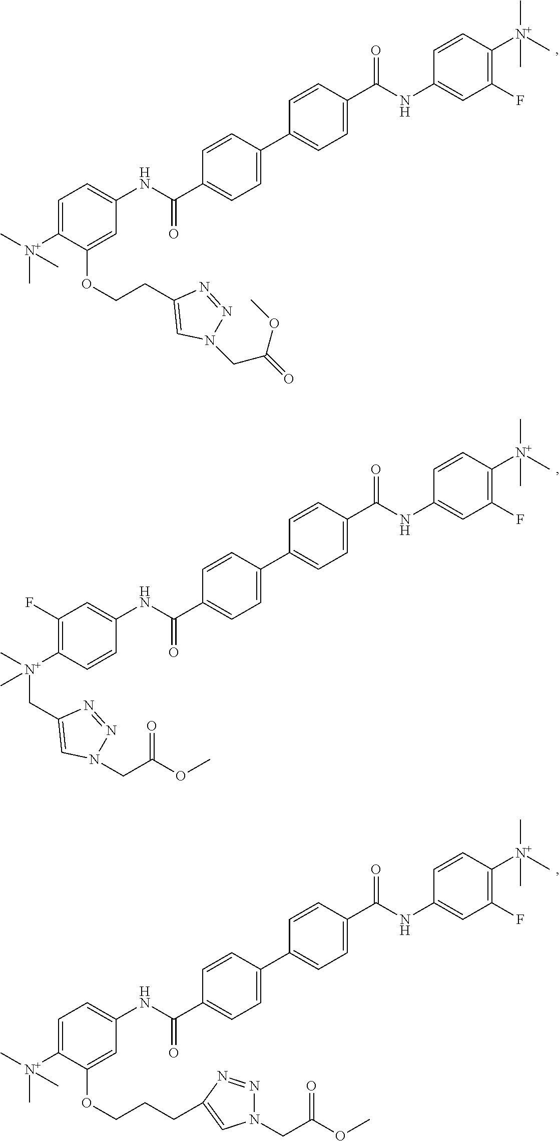

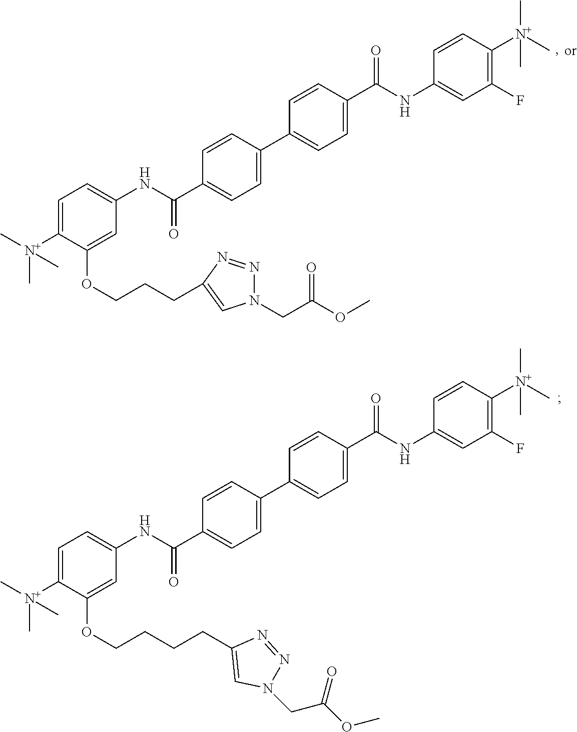

or a pharmaceutically acceptable salt thereof.

In yet another aspect, the present disclosure provides pharmaceutical compositions comprising:
(A) a compound described herein; and
(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical compositions are formulated as a unit dose.

In still another aspect, the present disclosure provides methods of treating a disease or disorder in a patient comprising administering to the patient in need thereof a therapeutically effective amount of a compound or composition described herein. In some embodiments, the disease or disorder is an infection. In some embodiments, the disease or disorder is an infection of a parasite. In some embodiments, the parasite is a bacterium. In some embodiments, the bacteria are a Gram positive bacteria. In other embodiments, the bacteria are a Gram negative bacteria. In some embodiments, the bacteria are resistant to one or more antibiotics. In some embodiments, the bacteria are resistant to methicillin. In some embodiments, the bacteria are E. coli or S. aureus. In some embodiments, the bacteria are methicillin resistant S. aureus. In some embodiments, the methods further comprise administering one or more additional antibiotics. In some embodiments, the methods comprise administering the compound or composition once. In other embodiments, the methods comprise administering the compound or composition one or more times.

In yet another aspect, the present disclosure may relate to compounds of formula IA and IB linked to a cell targeting moiety such as a biotin. The cell targeting moiety may be joined by a linker to the compound. The linker may comprise one or more alkyl chains, polymer chains such as polyethylene glycol, and one or more joining groups such as amines, amides, esters, carboxylic acids, or triazoles resulting from a Click reaction.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the survival of mice in a peritonitis model of infection.

### DESCRIPTION OF ILLUSTRATIVE EMBODIMENTS

The present disclosure provides compounds containing one or more quarternary ammonium ions. These compounds may further comprise one or more aromatic rings linking the two quarternary ammonium ions. These compounds may be used to treat one or more bacterial infections including an infection of a multi-drug resistant bacterial strain. Furthermore, these compounds may represent an improvement over those known in the art as the compounds may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties. These and more details will be discussed in more detail below.

### I. COMPOUNDS AND FORMULATIONS THEREOF

A. Compounds

The compounds of the present disclosure are shown, for example, above, in the summary of the invention section, the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the antibacterial compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the antibacterial compounds of the present disclsoure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the antibacterial compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The antibacterial compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the antibacterial compounds of the present disclosure can have the S or the R configuration. In some embodiments, the present compounds may contain two or more atoms which have a defined stereochemical orientation.

Chemical formulas used to represent the antibacterial compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the antibacterial compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, the antibacterial compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the antibacterial compound of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, the antibacterial compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. It is contemplated that the present methods include all different polymorphos of the compounds used herein. All solid forms of the antibacterial compound provided herein, including any solvates thereof are within the scope of the present invention.

B. Formulations

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of an antibacterial compound disclosed herein formulated with one or more excipients and/or drug carriers appropriate to the indicated route of administration. In some embodiments, the antibacterial compounds disclosed herein are formulated in a manner amenable for the treatment of human and/or veterinary patients. In some embodiments, formulation comprises admixing or combining one or more of the antibacterial compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the antibacterial compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the antibacterial compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water emulsions as well as conventional liposomes.

The antibacterial compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The antibacterial compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the antibacterial compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of an antibacterial compounds of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### III. INDICATIONS

B. Infections

i. Bacterial Pathogens

There are hundreds of bacterial pathogens in both the Gram-positive and Gram-negative families that cause significant illness and mortality around the word, despite decades of effort developing antibiotic agents. Indeed, antibiotic resistance is a growing problem in bacterial disease.

One of the bacterial diseases with highest disease burden is tuberculosis, caused by the bacterium Mycobacterium tuberculosis, which kills about 2 million people a year, mostly in sub-Saharan Africa. Some non-limiting examples of Mycobacterium tuberculosis antigens include recombinant Ag85A, Ag85B, ESAT6, TB10.4, or fragments thereof including those taught by Ottenhoff and Kaufmann, 2012, which is incorporated herein by reference. Pathogenic bacteria contribute to other globally important diseases, such as pneumonia, which can be caused by bacteria such as Streptococcus spp., Pseudomonas aeruginosa, Acinetobacter baumannii and Enterobacteriales, and foodborne illnesses, which can be caused by bacteria such as Shigella spp., Campylobacter spp., and Salmonella spp. Pathogenic bacteria also cause infections such as tetanus, typhoid fever, diphtheria, syphilis, and leprosy.

Conditionally pathogenic bacteria are only pathogenic under certain conditions, such as a wound facilitates entry of bacteria into the blood, or a decrease in immune function. For example, Staphylococcus spp. or Streptococcus spp. are also part of the normal human flora and usually exist on the skin or in the nose without causing disease, but can potentially cause skin infections, pneumonia, meningitis, and even overwhelming sepsis, a systemic inflammatory response producing shock, massive vasodilation and death. Some species of bacteria, such as Pseudomonas aeruginosa, Burkholderia cenocepacia, and Mycobacterium avium, are opportunistic pathogens and cause disease mainly in people suffering from immunosuppression or cystic fibrosis.

Other bacterial invariably cause disease in humans, such as obligate intracellular parasites (e.g., Chlamydophila, Ehrlichia, Rickettsia) that are capable of growing and reproducing only within the cells of other organisms. Still, infections with intracellular bacteria may be asymptomatic, such as during the incubation period. An example of intracellular bacteria is Rickettsiales. One species of Rickettsia prowazekii causes typhus, while another, R. rickettsia, causes Rocky Mountain spotted fever. Chlamydia, another phylum of obligate intracellular parasites, contains species that can cause pneumonia or urinary tract infection and may be involved in coronary heart disease. Mycobacterium spp., Brucella spp., Francisella spp., Legionella spp., and Listeria spp. can exist intracellularly, though they are facultative (not obligate) intracellular parasites. Cell membranes for these bacteria may be disrupted using the methods described herein.

### IV. THERAPIES

A. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of the antibacterial compounds of the present disclsoure formulated with one or more excipients and/or drug carriers appropriate to the indicated route of administration. In some embodiments, the antibacterial compounds disclosed herein are formulated in a manner amenable for the treatment of human and/or veterinary patients. In some embodiments, formulation comprises admixing or combining one or more of the antibacterial compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the antibacterial compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the antibacterial compounds disclosed herein may be coated in a material to protect the compound from the action of acids and other natural conditions which may inactivate the compound. To administer the active compound by other than parenteral administration, it may be necessary to coat the antibacterial compounds with, or co-administer the antibacterial compounds with, a material to prevent its inactivation. In some embodiments, the active antibacterial compounds may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The antibacterial compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The antibacterial compounds disclosed herein can be administered orally, for example, with an inert diluent or an assimilable edible carrier. The antibacterial compounds of the present disclosure and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the antibacterial compounds in the compositions and preparations may, of course, be varied. The amount of the therapeutic antibacterial compounds in such pharmaceutical formulations is such that a suitable dosage will be obtained.

The therapeutic antibiotic compound may also be administered topically to the skin, eye, ear, or mucosal membranes. Administration of the therapeutic antibiotic compound topically may include formulations of the antibiotic compound as a topical solution, lotion, cream, ointment, gel, foam, transdermal patch, or tincture. When the therapeutic antibiotic compound is formulated for topical administration, the antibiotic compound may be combined with one or more agents that increase the permeability of the antibiotic compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the antibiotic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic antibiotic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic antibiotic compound calculated to produce the desired therapeutic effect in association with the required pharmaceutical carrier. In some embodiments, the specification for the dosage unit forms of the disclosure are dictated by and directly dependent on (a) the unique characteristics of the therapeutic antibiotic compound and the particular therapeutic effect to be achieved, and (b) the limitations inherent in the art of compounding such a therapeutic antibiotic compound for the treatment of a selected condition in a patient. In some embodiments, active antibiotic compounds are administered at a therapeutically effective dosage sufficient to treat a condition associated with a condition in a patient. For example, the efficacy of a antibiotic compound can be evaluated in an animal model system that may be predictive of efficacy in treating the disease in a human or another animal.

In some embodiments, the effective dose range for the therapeutic antibiotic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a antibiotic compound of the present disclosure or composition comprising a antibiotic compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active antibiotic compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

B. Methods of Treatment

In particular, the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclsoure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, amount of the antibiotic compounds used is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the antibiotic compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

C. Combination Therapies

It is envisioned that the antibiotic compounds described herein may be used in combination therapies with one or more additional therapies or a compound which mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with a antibiotic compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the compounds described herein may precede or follow the other treatment by intervals ranging from minutes to weeks. One would generally ensure that a significant period of time did not expire between the times of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. A discussion of other potential therapies that may be used combination with the compounds of the present disclosure is presented elsewhere in this document.

### V. CHEMISTRY BACKGROUND

In some aspects, antibiotic compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

A. Process Scale-Up

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof, “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

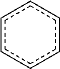

covers, for example,

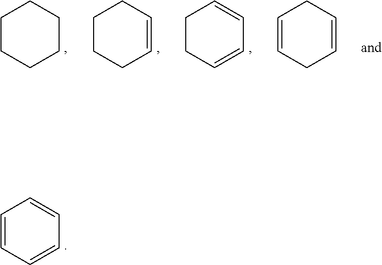

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

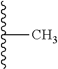

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

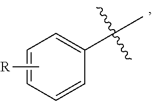

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

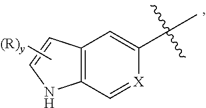

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C₁₋₄-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

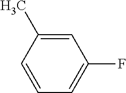

is also taken to refer to

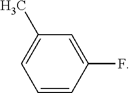

Aromatic compounds may also be depicted using a circle to represent the delocalized nature of the electrons in the fully conjugated cyclic π system, two non-limiting examples of which are shown below:

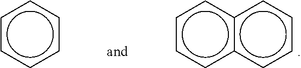

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

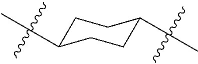 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

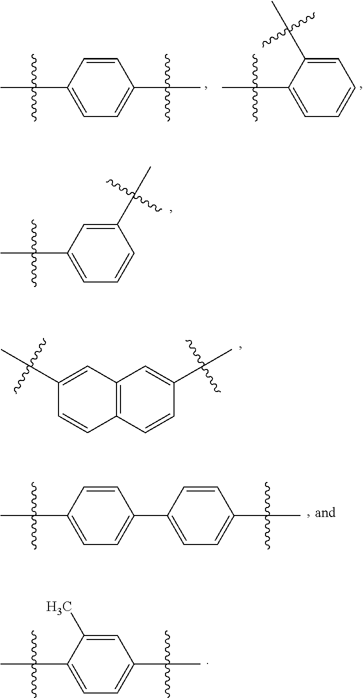

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group -alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alkylsulfonyl” refers to the group: —S(O)₂R, in which R is an alkyl. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of ±10% of the reported value, preferably a value of ±5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “EC₅₀” refers to an amount that is an effective concentration to results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Preparation of Compounds

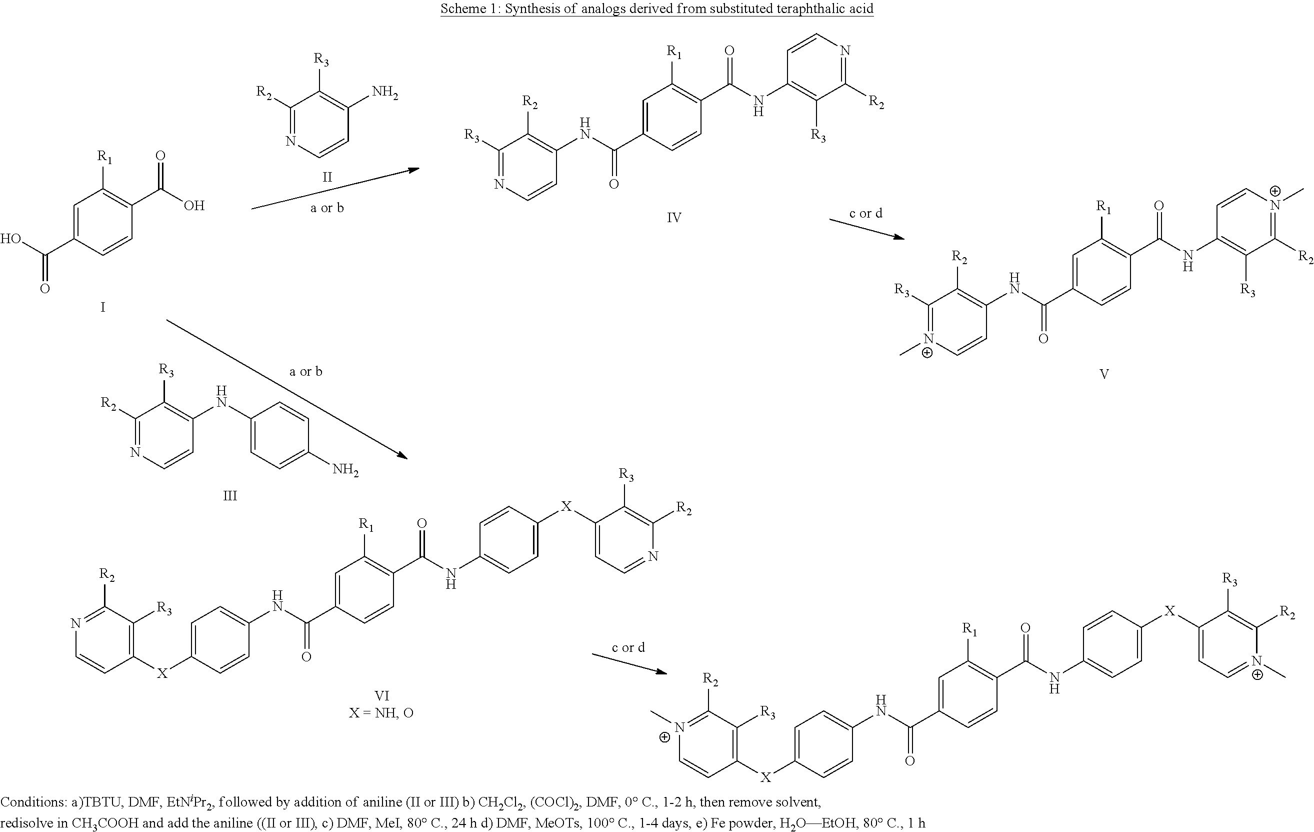

Compounds according to general structures V and VII were prepared from substituted terephthalic acids and various substituted anilines of general structures II or III according to the general procedure outlined in Scheme 1. Starting from the terephthalic acids, I, addition of the various substituted anilines, II or III was achieved either via a standard amide coupling procedure using coupling agents such as TBTU gave the amide intermediates IV or VI. Alternatively, the conversion to the diacid chloride with oxalyl chloride followed by addition of the appropriate anilne also gave amide intermediates of type IV and VI. These could then be converted to the N-methyl pyridinium salts of general structures V and VIII via addition of methyl iodide and heating overnight. Compounds with nitro groups could be converted to the amino compound via reduction such as with iron powder.

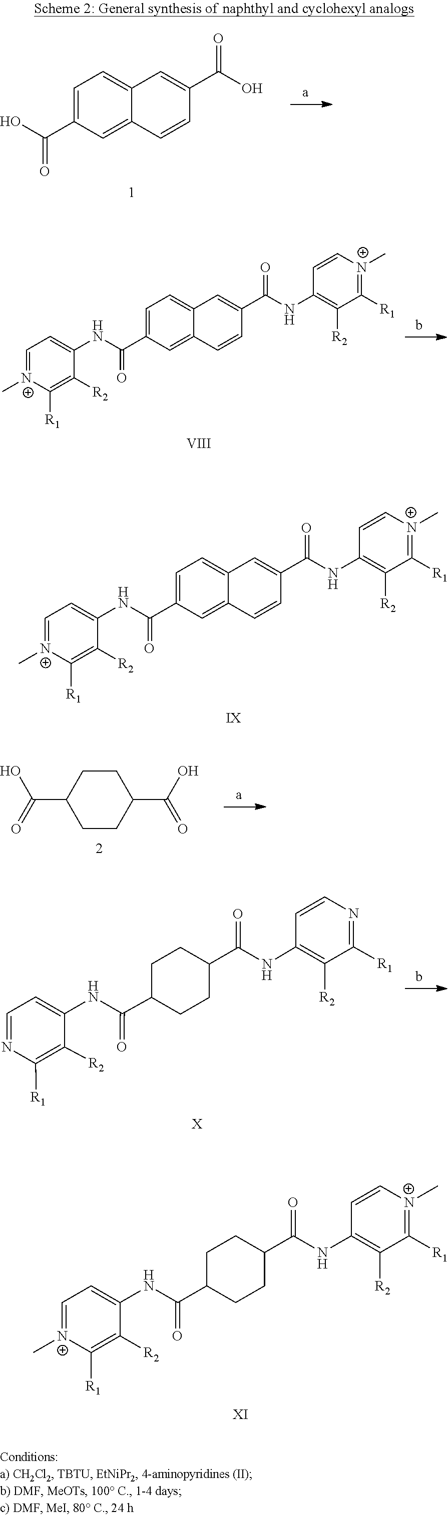

Analogs according to general structures VIII-XI were prepared according to the procedures shown in Scheme 2. Coupling of either diacid 1 or 2 with substituted anilines II or III with standard amide coupling reagents such as TBTU gave rise to the diamides VIII and X. The N-methyl pyridinium compounds could then be prepared via heating in the presence of a methylating agent such as methyl iodide or methyl tosylate.

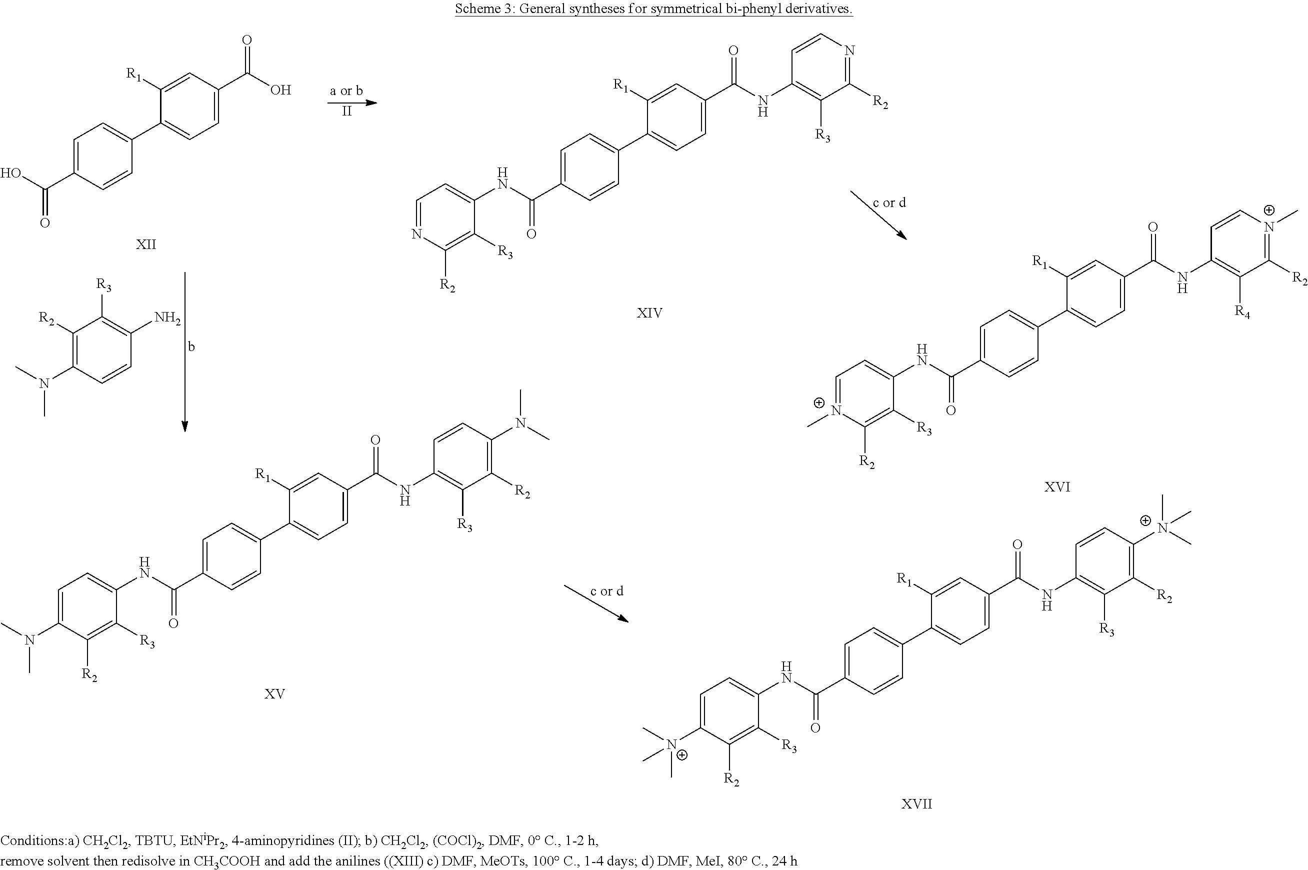

Substituted bi-phenyl diacids of general structure XIV could be prepared via from the biphenyl diacids, XII and anilines of general structure II via standard coupling with reagents such as TBTU or T3P. Alternatively, the di-acids could be converted to the diacid chloride and then reacted with anilines of structure II. Addition of a methylating agent such as methyl iodide or methyl tosylate to XIV and heating gave rise to the methylpyridinium compounds such as XVI. Reaction of bi-phenyl diacid XII with the dimethylamino-substituted anilines of general structure XIII gave rise to the diamide structures of XV. Heating XV in the presence of a methylating agent such as methyl iodide or methyl tosylate gave right the quaternary amine compounds, XVII.

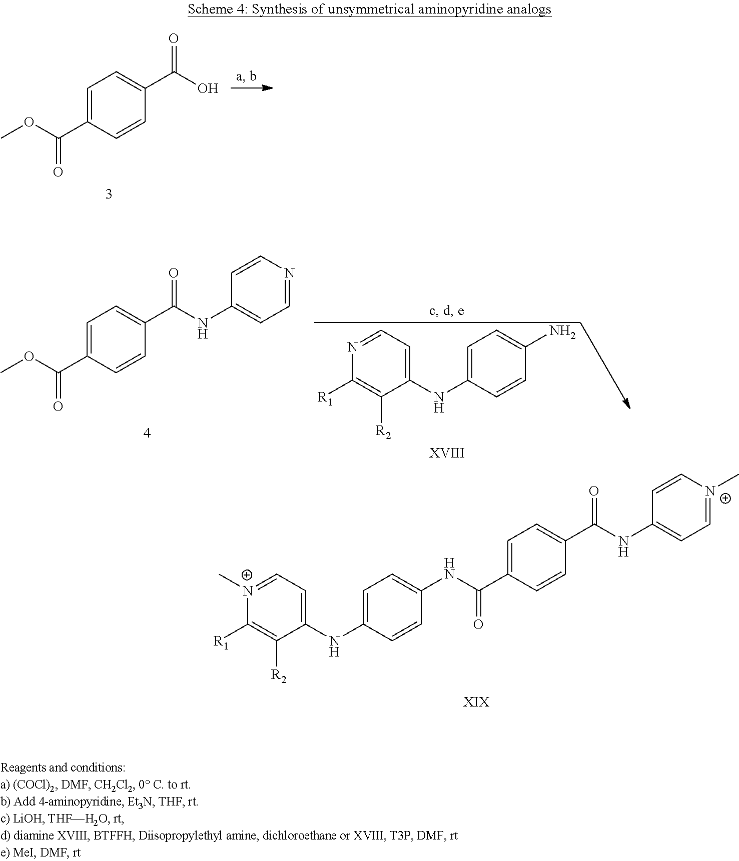

Compounds according to general structure XIX were prepared as outlined in Scheme 4. Starting from known acid 3, conversion to the acid chloride followed by addition of 4-aminopyridine gave amide compound 4. Hydrolysis of the ester followed by amide type couplings, such as T3P in DMF or BTFFH in dichloroethane, of the resultant acid with a various substituted 4-pyridyl-1,4-diaminophenyl compounds of general structure XVIII followed by addition of methyl iodide gave rise to the methylpyridinium compounds of type XIX.

### Experimental Section

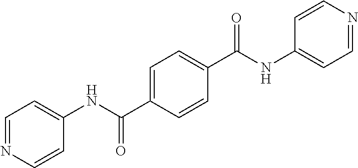 [SMILES: O=C(Nc1ccncc1)c1ccc(C(=O)Nc2ccncc2)cc1]

### Example 1

N¹,N⁴-di(pyridin-4-yl)terephthalamide (Example 1) (General Procedure A): terephthaloyl chloride (306 mg, 1.5 mmol) and TBTU (483 mg, 1.5 mmol) were mixed in dry DMF (2.0 mL, 0.75 M) then added DIPEA (969 mg, 7.5 mmol) and stirred for 10-20 minutes at RT. After 10-20 minutes 4-aminopyridine (285 mg, 3.0 mmol) was added under argon and stirred at RT for 1 hour-1 day. Ether was added to the reaction mixture, and it was filtered to collect the pure product (white solid, 0.201 g, 84%). ¹H NMR (400 MHz, DMSO) δ 10.81 (s, 2H), 8.52 (dd, J=4.9, 1.5 Hz, 4H), 8.13 (s, 4H), 7.83 (dd, J=4.9, 1.5 Hz, 4H). LCMS: 1.455 min, MS: ES+319.1.

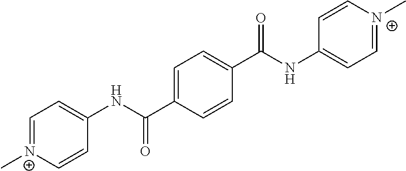

### Example 2

4,4′-(terephthaloylbis(azanediyl))bis(1-methylpyridin-1-ium) (Example 2) (General procedure B): N¹,N⁴-di(pyridin-4-yl)terephthalamide (100 mg, 0.31 mmol) and iodomethane (681 mg, 4.8 mmol, 15 equiv.) were mixed in dry DMF (1.5 mL, 0.2 M) under argon and stirred at 82° C. for 1 day. The reaction mixture was dissolved in minimum amount of DMSO and purified on a 50 g C18 reversed-phase column (acetonitrile/water). Product was isolated as a TFA salt. (white solid, 0.059 g, 33%). ¹H NMR (400 MHz, DMSO) δ 11.82 (s, 2H), 8.80 (d, J=7.2 Hz, 4H), 8.32 (d, J=6.3 Hz, 4H), 8.23 (s, 4H), 4.22 (s, 6H). ¹⁹F NMR (376 MHz, DMSO-D₆) δ −74.32. LCMS: 1.462 min, MS: ES+347.1.

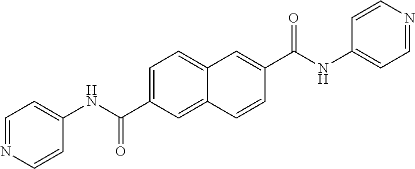 [SMILES: O=C(Nc1ccncc1)c1ccc2cc(C(=O)Nc3ccncc3)ccc2c1]

### Example 3

N²,N⁶-di(pyridin-4-yl)naphthalene-2,6-dicarboxamide (Example 3) (General procedure C): A solution of 2,6-naphthalenedicarboxylic acid (216 mg, 1.0 mmol) in 2.0 mL of dichloromethane was cooled to 0° C. using an ice bath. To this solution was added sequentially a catalytic amount (˜1-2 drops) of DMF followed by a 2.0 M solution of oxalyl chloride (2.0 mL) in dichloromethane. The resulting mixture was stirred overnight, allowing the temperature to gradually warm to rt. The resulting mixture was concentrated down to dryness on a rotary evaporator and then dried under vacuum for 2 h. The resulting solid and TBTU (321 mg, 1.0 mmol) were mixed in dry DMF (2.0 mL, 0.5 M) then added DIPEA (646 mg, 5 mmol) and stirred for 10-30 minutes at RT. After 10-30 minutes 4-aminopyridine (188 mg, 2.0 mmol) was added under argon and stirred at RT for 1 hour-1 day. The reaction mixture was dissolved in minimum amount of DMSO and purified on a 50 g C18 reversed-phase column (acetonitrile/water). Product was isolated as a TFA salt (white solid, 0.202 g, 42%). ¹H NMR (400 MHz, DMSO) δ 11.65 (s, 2H), 8.77 (d, J=7.0 Hz, 6H), 8.33 (d, J=8.6 Hz, 2H), 8.26 (d, J=7.1 Hz, 4H), 8.16 (dd, J=8.5, 1.5 Hz, 2H). ¹⁹F NMR (376 MHz, DMSO-D₆) δ −73.85. LCMS: 1.691 min, MS: ES+368.9.

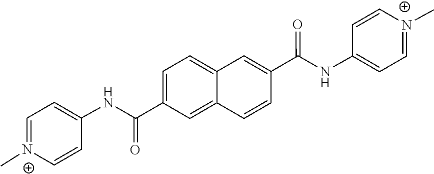

### Example 4

4,4′-((naphthalene-2,6-dicarbonyl)bis(azanediyl))bis(1-methylpyridin-1-ium) (Example 4) (General Procedure D): N²,N⁶-di(pyridin-4-yl)naphthalene-2,6-dicarboxamide (50 mg, 0.135 mmol) was dissolved in ammonia solution (7 N in methanol) (2 ml) and stirred for 1 hour at room temperature. The ammonia was then allowed to evaporate off and resulting mixture was dried under high vacuum over night before being used for the methylation reaction. To the resulting residue was added methyl p-toluenesulfonate (376 mg, 2.0 mmol, 7.5-10 equiv.) and the mixture was then dissolved in dry DMF (0.7 mL, 0.2 M) under argon and stirred at 100° C. for 1 to 4 days. The reaction mixture was dissolved in minimum amount of DMSO and purified on a 50 g C18 reversed-phase column (acetonitrile/water). Product was isolated as a TFA salt. Isolated product was dissolved in water (2.5 mL) and stirred with ion-exchange resin (1-X₄, BioRad AG, 200-400 chloride form) (0.35 g) for 1 hour. The resulting slurry was loaded onto the 1 g column of the same resin. The column was eluted with water and fractions containing the compound were combined and evaporated to dryness to get the pure chloride salt (white solid, 0.043, 67%). ¹H NMR (400 MHz, D₂O) δ 8.65-8.53 (m, 6H), 8.29-8.18 (m, 6H), 8.05 (d, J=8.6 Hz, 2H), 4.27 (s, 6H). LCMS: 1.697 min, MS: ES+397.9.

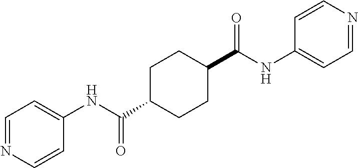 [SMILES: O=C(Nc1ccncc1)[C@H]1CC[C@H](C(=O)Nc2ccncc2)CC1]

### Example 5

(E)-N¹,N⁴-di(pyridin-4-yl)cyclohexane-1,4-dicarboxamide (Example 5): Prepared from (E)-cyclohexane-1,4-dicarboxylic acid and pyridin-4-amine following the procedure C (white solid, 0.157 g, 24%.). ¹H NMR (400 MHz, DMSO) δ 11.14 (s, 2H), 8.63 (d, J=6.7 Hz, 4H), 7.96 (d, J=6.6 Hz, 4H), 3.65-3.58 (m, 1H), 2.09-1.91 (m, 4H), 1.59-1.37 (m, 4H), 1.25 (dd, J=13.0, 6.6 Hz, 1H). ¹⁹F NMR (376 MHz, DMSO-D₆) δ −73.64. LCMS: 1.444 min, MS: ES+325.0.

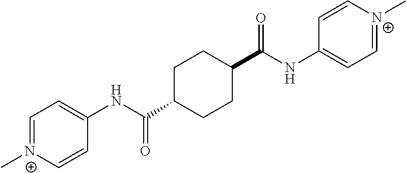

### Example 6

4,4′-((E)-cyclohexanedicarbonylbis(azanediyl))bis(1-methylpyridin-1-ium) (Example 6): Prepared from (E)-N¹,N⁴-di(pyridin-4-yl)cyclohexane-1,4-dicarboxamide and methyl p-toluenesulfonate following the procedure D (white solid, 0.021 g, 54%.). ¹H NMR (400 MHz, D₂O) δ 8.54 (d, J=7.3 Hz, 4H), 8.09 (d, J=7.4 Hz, 4H), 4.23 (s, 6H), 2.63 (p, J=9.4 Hz, 2H), 2.20-2.06 (m, 4H), 1.71-1.55 (m, 4H). LCMS: 1.436 min, MS: ES+354.0.

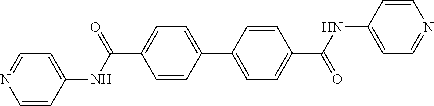 [SMILES: O=C(Nc1ccncc1)c1ccc(-c2ccc(C(=O)Nc3ccncc3)cc2)cc1]

### Example 7

N⁴,N^{4′}-di(pyridin-4-yl)-[1,1′-biphenyl]-4,4′-dicarboxamide (Example 7): Prepared from biphenyl-4,4′-dicarboxylic acid and pyridin-4-amine following the procedure C (white solid, 0.106 g, 21%.). ¹H NMR (400 MHz, DMSO) δ 11.49 (s, 2H), 8.76 (d, J=7.1 Hz, 4H), 8.28 (d, J=7.2 Hz, 4H), 8.18 (d, J=8.5 Hz, 4H), 8.04 (d, J=8.5 Hz, 4H). ¹⁹F NMR (376 MHz, DMSO-D₆) δ −73.90. LCMS: 1.786 min, MS: ES+395.9.

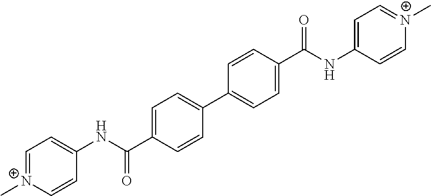

### Example 8

4,4′-(([1,1′-biphenyl]-4,4′-dicarbonyl)bis(azanediyl))bis(1-methylpyridin-1-ium)) (Example 8): Prepared from N⁴,N^{4′}-di(pyridin-4-yl)-[1,1′-biphenyl]-4,4′-dicarboxamide and methyl p-toluenesulfonate following the procedure D (white solid, 0.025 g, 43%.). ¹H NMR (400 MHz, D₂O) δ 8.53 (d, J=7.3 Hz, 4H), 8.16 (d, J=7.3 Hz, 4H), 7.91 (d, J=8.4 Hz, 4H), 7.80 (d, J=8.4 Hz, 4H), 4.19 (s, 6H). LCMS: 1.839 min, MS: ES+424.2.

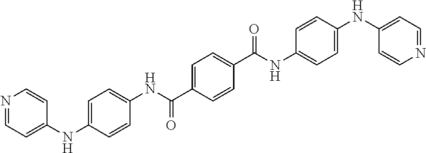 [SMILES: O=C(Nc1ccc(Nc2ccncc2)cc1)c1ccc(C(=O)Nc2ccc(Nc3ccncc3)cc2)cc1]

### Example 9

N¹,N⁴-bis(4-(pyridin-4-ylamino)phenyl)terephthalamide (Example 9) (General Procedure E): N¹-(pyridin-4-yl)benzene-1,4-diamine (47 mg, 0.25 mmol) was dissolved in glacial acetic acid (0.5 mL, 0.2 M) before being added to the terephthaloyl chloride (20 mg, 0.1 mmol). The resulting mixture was stirred for 1 hour at room temperature. The solvent was removed, and crude product was dissolved in minimum amount of DMSO and purified on a 50 g C18 reversed-phase column (acetonitrile/water). Product was isolated as a TFA salt (Flesh colored solid, 0.020 g, 27%.) ¹H NMR (400 MHz, DMSO) δ 13.53 (s, 2H), 10.57 (s, 2H), 10.40 (s, 2H), 8.27 (d, J=7.1 Hz, 4H), 8.13 (s, 4H), 7.93 (d, J=8.8 Hz, 4H), 7.36 (d, J=8.8 Hz, 4H), 7.07 (d, J=7.1 Hz, 4H). ¹⁹F NMR (376 MHz, DMSO-D₆) δ −73.45. LCMS: 1.818 min, MS: ES+501.2.

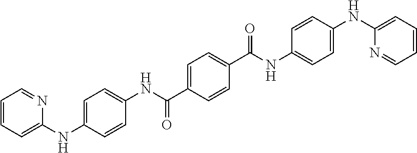 [SMILES: O=C(Nc1ccc(Nc2ccccn2)cc1)c1ccc(C(=O)Nc2ccc(Nc3ccccn3)cc2)cc1]

### Example 10

N¹,N⁴-bis(4-(pyridin-2-ylamino)phenyl)terephthalamide (Example 10): Prepared from terephthaloyl chloride and N¹-(pyridin-2-yl)benzene-1,4-diamine following the procedure E (light yellow solid, 0.025 g, 35%.). ¹H NMR (400 MHz, DMSO) δ 10.39 (s, 2H), 9.59 (s, 2H), 8.11 (s, 4H), 8.08 (d, J=4.4 Hz, 2H), 7.76 (dd, J=19.4, 8.2 Hz, 6H), 7.57 (d, J=8.8 Hz, 4H), 6.95 (d, J=8.7 Hz, 2H), 6.88-6.78 (m, 2H). ¹⁹F NMR (376 MHz, DMSO-D₆) δ −74.34. LCMS: 1.976 min, MS: ES+501.2.

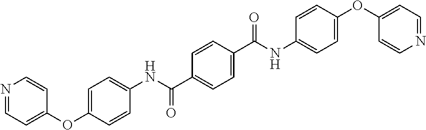 [SMILES: O=C(Nc1ccc(Oc2ccncc2)cc1)c1ccc(C(=O)Nc2ccc(Oc3ccncc3)cc2)cc1]

### Example 11

N¹,N⁴-bis(4-(pyridin-4-yloxy)phenyl)terephthalamide (Example 11): Prepared from terephthaloyl chloride and 4-(pyridin-4-yloxy)aniline following the procedure E (white solid, 0.025 g, 34%.). ¹H NMR (400 MHz, DMSO) δ 10.61 (s, 2H), 8.71 (d, J=6.4 Hz, 4H), 8.14 (s, 4H), 7.98 (d, J=9.0 Hz, 4H), 7.34-7.32 (m, 8H). ¹⁹F NMR (376 MHz, DMSO-D₆) d −74.04. LCMS: 1.928 min, MS: ES+503.1.

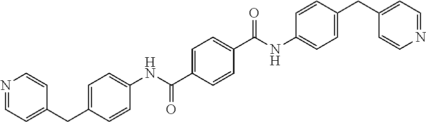 [SMILES: O=C(Nc1ccc(Cc2ccncc2)cc1)c1ccc(C(=O)Nc2ccc(Cc3ccncc3)cc2)cc1]

### Example 12

N¹,N⁴-bis(4-(pyridin-4-ylmethyl)phenyl)terephthalamide (Example 12): Prepared from terephthaloyl chloride and 4-(pyridin-4-ylmethyl)aniline following the procedure E (white solid, 0.095 g, 65%.). ¹H NMR (400 MHz, DMSO) δ 10.40 (s, 2H), 8.66 (d, J=4.3 Hz, 4H), 8.08 (s, 4H), 7.75 (d, J=8.5 Hz, 4H), 7.61 (d, J=5.7 Hz, 4H), 7.29 (d, J=8.5 Hz, 4H), 4.11 (s, 4H). ¹⁹F NMR (376 MHz, DMSO-D₆) d −73.85. LCMS: 1.8600 min, MS: ES+499.2.

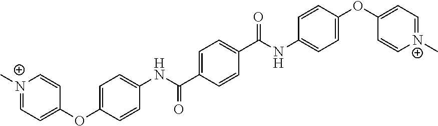

### Example 13

4,4′-(((terephthaloylbis(azanediyl))bis(4,1-phenylene))bis(oxy))bis(1-methylpyridin-1-ium) (Example 13): Prepared from N¹,N⁴-bis(4-(pyridin-4-yloxy)phenyl)terephthalamide and methyl p-toluenesulfonate following the procedure D (white solid, 0.021 g, 91%.). ¹H NMR (400 MHz, D₂O) δ 8.56 (d, J=7.2 Hz, 4H), 8.00 (s, 4H), 7.70 (d, J=8.8 Hz, 4H), 7.41 (d, J=7.3 Hz, 4H), 7.31 (d, J=8.9 Hz, 4H), 4.21 (s, 6H). LCMS: 1.949 min, MS: ES+266.1.

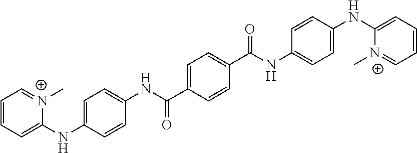 [SMILES: C[n+]1ccccc1Nc1ccc(NC(=O)c2ccc(C(=O)Nc3ccc(Nc4cccc[n+]4C)cc3)cc2)cc1]

### Example 14

2,2′-(((terephthaloylbis(azanediyl))bis(4,1-phenylene))bis(azanediyl))bis(1-methylpyridin-1-ium) (Example 14): Prepared from N¹,N⁴-bis(4-(pyridin-2-ylamino)phenyl)terephthalamide and methyl p-toluenesulfonate following the procedure D (white solid, 0.051 g, 84%.). ¹H NMR (400 MHz, D₂O) δ 8.02 (d, J=6.6 Hz, 2H), 7.97 (d, J=7.3 Hz, 4H), 7.87 (t, J=8.1 Hz, 2H), 7.66 (d, J=8.4 Hz, 4H), 7.37 (d, J=8.5 Hz, 4H), 7.11 (d, J=9.0 Hz, 2H), 7.01 (t, J=6.9 Hz, 2H), 3.99 (s, 6H). LCMS: 1.877 min, MS: ES+530.2.

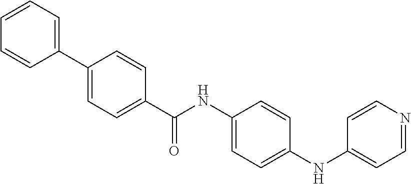 [SMILES: O=C(Nc1ccc(Nc2ccncc2)cc1)c1ccc(-c2ccccc2)cc1]

### Example 15

N-(4-(pyridin-4-ylamino)phenyl)-[1,1′-biphenyl]-4-carboxamide (Example 15): Prepared from biphenyl-4-carbonyl chloride and N¹-(pyridin-4-yl)benzene-1,4-diamine following the procedure E (tan colored solid, 0.075 g, 78%.). ¹H NMR (400 MHz, DMSO) δ 13.49 (s, 1H), 10.46 (s, 1H), 10.42 (s, 1H), 8.27 (d, J=6.7 Hz, 2H), 8.07 (d, J=8.1 Hz, 2H), 7.94 (d, J=8.6 Hz, 2H), 7.86 (d, J=8.1 Hz, 2H), 7.77 (d, J=7.8 Hz, 2H), 7.52 (t, J=7.5 Hz, 2H), 7.44 (t, J=7.3 Hz, 1H), 7.35 (d, J=8.6 Hz, 2H), 7.06 (d, J=6.7 Hz, 2H). ¹⁹F NMR (376 MHz, DMSO-D₆) d −73.46. LCMS: 2.377 min, MS: ES+366.2.

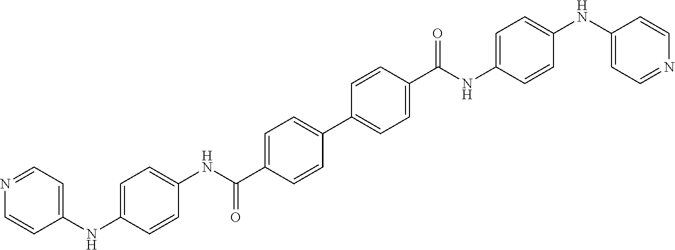 [SMILES: O=C(Nc1ccc(Nc2ccncc2)cc1)c1ccc(-c2ccc(C(=O)Nc3ccc(Nc4ccncc4)cc3)cc2)cc1]

### Example 16

N⁴,N^{4′}-bis(4-(pyridin-4-ylamino)phenyl)-[1,1′-biphenyl]-4,4′-dicarboxamide (Example 16): Prepared from [1,1′-biphenyl]-4,4′-dicarbonyl dichloride and N¹-(pyridin-4-yl)benzene-1,4-diamine following the procedure E (tan colored solid, 0.034 g, 42%.). ¹H NMR (400 MHz, DMSO) δ 13.54 (s, 2H), 10.51 (s, 2H), 10.46 (s, 2H), 8.28 (d, J=6.8 Hz, 4H), 8.13 (d, J=8.2 Hz, 4H), 7.96 (t, J=7.9 Hz, 8H), 7.36 (d, J=8.6 Hz, 4H), 7.07 (d, J=6.8 Hz, 4H). ¹⁹F NMR (376 MHz, DMSO-D₆) d −73.49. LCMS: 2.116 min, MS: ES+577.3.

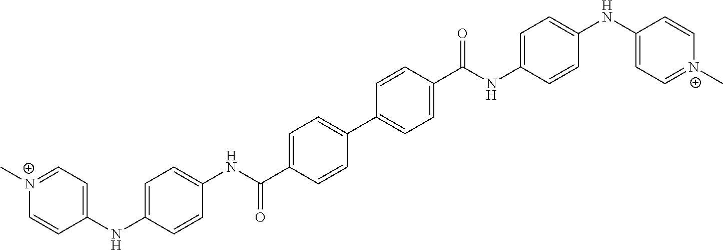

### Example 17

4,4′-(((([1,1′-biphenyl]-4,4′-dicarbonyl)bis(azanediyl))bis(4,1-phenylene))bis(azanediyl))bis(1-methylpyridin-1-ium) (Example 17): Prepared from N⁴,N^{4′}-bis(4-(pyridin-4-ylamino)phenyl)-[1,1′-biphenyl]-4,4′-dicarboxamide (Example 16) and methyl p-toluenesulfonate following the procedure D (yellow solid, 0.034 g, 91%.). ¹H NMR (400 MHz, DMSO) δ 10.86 (s, 1H), 10.80 (s, 1H), 10.59 (s, 1H), 10.58 (s, 1H), 8.27 (d, J=7.3 Hz, 4H), 8.15 (d, J=8.3 Hz, 4H), 7.97 (dd, J=8.7, 2.2 Hz, 8H), 7.35 (d, J=8.8 Hz, 4H), 7.15 (d, J=6.1 Hz, 4H), 3.95 (s, 6H). LCMS: 2.243 min, MS: ES+605.3.

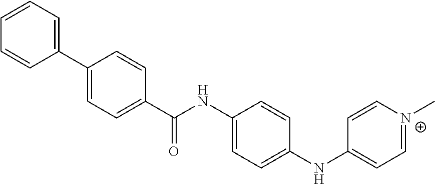 [SMILES: C[n+]1ccc(Nc2ccc(NC(=O)c3ccc(-c4ccccc4)cc3)cc2)cc1]

### Example 18

4-((4-([1,1′-biphenyl]-4-ylcarboxamido)phenyl)amino)-1-methylpyridin-1-ium (Example 18): Prepared from N-(4-(pyridin-4-ylamino)phenyl)-[1,1′-biphenyl]-4-carboxamide (Example 15) and methyl p-toluenesulfonate following the procedure D (white solid, 0.046 g, 74%.). ¹H NMR (400 MHz, MeOD) δ 8.14 (d, J=7.0 Hz, 2H), 8.04 (d, J=8.1 Hz, 2H), 7.88 (d, J=8.6 Hz, 2H), 7.78 (d, J=8.0 Hz, 2H), 7.69 (d, J=7.9 Hz, 2H), 7.48 (t, J=7.6 Hz, 2H), 7.39 (t, J=7.3 Hz, 1H), 7.34 (d, J=8.5 Hz, 2H), 7.09 (d, J=7.0 Hz, 2H), 3.98 (s, 3H). LCMS: 2.446 min, MS: ES+381.2.

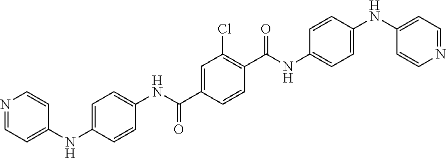 [SMILES: O=C(Nc1ccc(Nc2ccncc2)cc1)c1ccc(C(=O)Nc2ccc(Nc3ccncc3)cc2)c(Cl)c1]

### Example 19

### 2-chloro-N¹,N⁴-bis(4-(pyridin-4-ylamino)phenyl)terephthalamide (Example 19) (General Procedure F)

Step 1: A solution of 2-chloroterephthalic acid (40 mg, 0.2 mmol) in 1.0 mL of dichloromethane was cooled to 0° C. using an ice bath. To this solution was added sequentially a catalytic amount (˜1-2 drops) of DMF followed by a 2.0 M solution of oxalyl chloride (1.0 mL) in dichloromethane. The resulting mixture was stirred overnight, allowing the temperature to gradually warm to rt. The resulting mixture was concentrated down to dryness on a rotary evaporator and then dried under vacuum for 2 h to get the pure 2-chloroterephthaloyl chloride.

Step 2: N¹-(pyridin-4-yl)benzene-1,4-diamine (94 mg, 0.5 mmol) was dissolved in glacial acetic acid (1.0 mL, 0.2 M) and stirred at rt for 5 min. This solution was then added via sytinge to the 2-chloroterephthaloyl chloride (48 mg, 0.2 mmol) in a 2-dram vial. The resulting mixture was stirred for 30 minutes at room temperature. The solvent was removed, and crude product was dissolved in minimum amount of DMSO and purified on a 50 g C18 reversed-phase column (acetonitrile/water). Product was isolated as a TFA salt. (tan colored solid, 0.039 g, 26%). ¹H NMR (400 MHz, DMSO) δ 13.58 (s, 2H), 10.82 (s, 1H), 10.62 (s, 1H), 10.46 (s, 2H), 8.28 (d, J=4.4 Hz, 4H), 8.18 (s, 1H), 8.06 (d, J=8.1 Hz, 1H), 7.93 (d, J=8.6 Hz, 2H), 7.85 (d, J=8.6 Hz, 2H), 7.79 (d, J=7.9 Hz, 1H), 7.36 (dd, J=8.7, 2.6 Hz, 4H), 7.07 (d, J=6.8 Hz, 4H). ¹⁹F NMR (376 MHz, DMSO-D₆) δ −73.49. LCMS: 1.912 min, MS: ES+536.2.

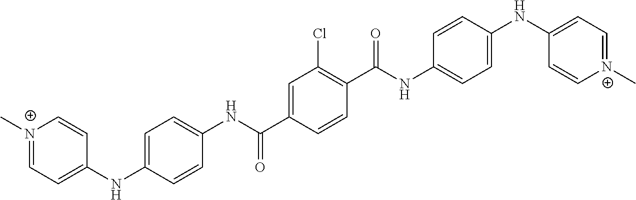

### Example 20

4,4′-((((2-chloroterephthaloyl)bis(azanediyl))bis(4,1-phenylene))bis(azanediyl))bis(1-methylpyridin-1-ium) (Example 20): Prepared from 2-chloro-N¹,N⁴-bis(4-(pyridin-4-ylamino)phenyl)terephthalamide and methyl p-toluenesulfonate following the procedure D (white solid, 0.039 g, 70%.). ¹H NMR (400 MHz, DMSO) δ 10.86 (s, 1H), 10.82 (s, 1H), 10.79 (s, 1H), 10.72 (s, 1H), 8.27 (d, J=5.8 Hz, 4H), 8.21 (s, 1H), 8.09 (d, J=8.0 Hz, 1H), 7.96 (d, J=8.8 Hz, 2H), 7.86 (d, J=8.8 Hz, 2H), 7.78 (d, J=7.9 Hz, 1H), 7.36 (dd, J=8.9, 2.9 Hz, 4H), 7.15 (d, J=4.8 Hz, 4H), 3.96 (s, 6H). LCMS: 2.121 min, MS: ES+282.2.

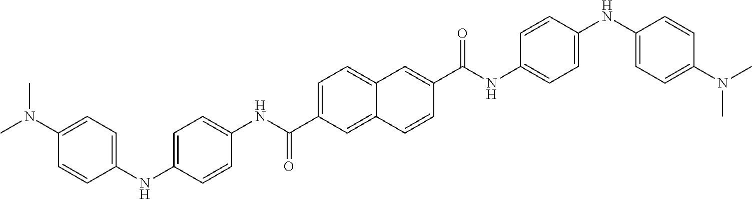

### Example 21

N²,N⁶-bis(4-(pyridin-4-ylamino)phenyl)naphthalene-2,6-dicarboxamide (Example 21): Prepared from naphthalene-2,6-dicarbonyl dichloride and N¹-(pyridin-4-yl)benzene-1,4-diamine following the procedure F (tan colored solid, 0.010 g, 13%.). ¹H NMR (400 MHz, DMSO) δ 13.55 (s, 2H), 10.71 (s, 2H), 10.46 (s, 2H), 8.67 (s, 2H), 8.27 (t, J=8.3 Hz, 6H), 8.13 (d, J=8.6 Hz, 2H), 7.97 (d, J=8.6 Hz, 4H), 7.38 (d, J=8.6 Hz, 4H), 7.08 (d, J=6.8 Hz, 4H). ¹⁹F NMR (376 MHz, DMSO-D₆) d −73.49. LCMS: 2.053 min, MS: ES+551.3.

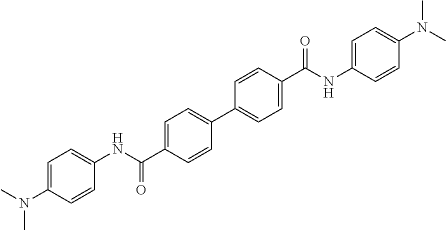

### Example 22

N⁴,N^{4′}-bis(4-(dimethylamino)phenyl)-[1,1′-biphenyl]-4,4′-dicarboxamide (Example 22): Prepared from [1,1′-biphenyl]-4,4′-dicarbonyl dichloride and N,N-dimethyl-p-phenylenediamine following the procedure C (light green solid, 0.078 g, 81%.). ¹H NMR (400 MHz, DMSO) δ 10.06 (s, 2H), 8.08 (d, J=8.1 Hz, 4H), 7.91 (d, J=8.2 Hz, 4H), 7.60 (d, J=8.8 Hz, 4H), 6.74 (d, J=8.9 Hz, 4H), 2.88 (s, 12H). LCMS: 2.545 min, MS: ES+479.3.

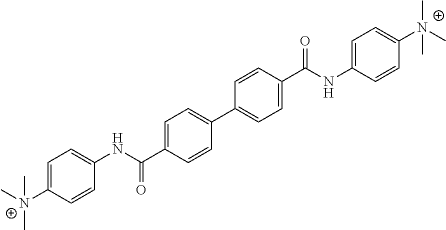

### Example 23

4,4′-(([1,1′-biphenyl]-4,4′-dicarbonyl)bis(azanediyl))bis(N,N,N-trimethylbenzenaminium) (Example 23): Prepared from example 22 and methyl p-toluenesulfonate following the procedure D (tan colored solid, 0.063 g, 72%.). ¹H NMR (400 MHz, DMSO) δ 10.92 (s, 2H), 8.22 (d, J=8.4 Hz, 4H), 8.11 (d, J=9.4 Hz, 4H), 7.98 (dd, J=12.5, 9.0 Hz, 8H), 3.63 (s, 18H). LCMS: 1.875 min, MS: ES+254.2.

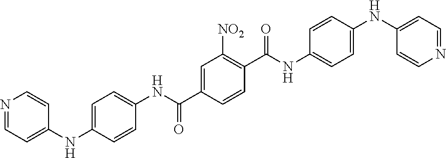 [SMILES: O=C(Nc1ccc(Nc2ccncc2)cc1)c1ccc(C(=O)Nc2ccc(Nc3ccncc3)cc2)c([N+](=O)[O-])c1]

### Example 24

2-nitro-N¹,N⁴-bis(4-(pyridin-4-ylamino)phenyl)terephthalamide (Example 24): Prepared from 2-nitroterephthaloyl dichloride and N¹-(pyridin-4-yl)benzene-1,4-diamine following the procedure F (yellow solid, 0.087 g, 28%). ¹H NMR (400 MHz, DMSO) δ 13.80 (s, 2H), 10.95 (s, 1H), 10.82 (s, 1H), 10.06 (s, 2H), 8.74 (d, J=1.4 Hz, 1H), 8.53-8.44 (m, 1H), 8.24 (dd, J=6.9, 3.0 Hz, 4H), 7.98 (d, J=7.9 Hz, 1H), 7.91 (d, J=8.8 Hz, 2H), 7.77 (d, J=8.8 Hz, 2H), 7.33 (dd, J=8.8, 5.0 Hz, 4H), 7.04 (d, J=5.5 Hz, 4H). ¹⁹F NMR (376 MHz, DMSO-D₆) δ −73.45. LCMS: 2.563 min, MS: ES+546.2.

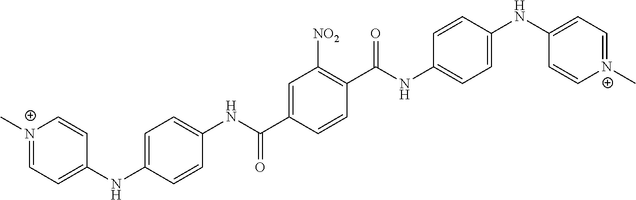

### Example 25

4,4′-((((2-nitroterephthaloyl)bis(azanediyl))bis(4,1-phenylene))bis(azanediyl))bis(1-methylpyridin-1-ium) (Example 25): Prepared from example 24 and methyl p-toluenesulfonate following the procedure D (yellow solid, 0.053 g, 77%). ¹H NMR (400 MHz, DMSO) δ 11.05 (s, 1H), 10.93 (s, 1H), 10.78 (s, 1H), 10.74 (s, 1H), 8.77 (d, J=1.4 Hz, 1H), 8.52 (dd, J=7.9, 1.4 Hz, 1H), 8.28 (dd, J=7.3, 3.1 Hz, 4H), 7.99 (t, J=7.9 Hz, 3H), 7.83 (d, J=8.8 Hz, 2H), 7.38 (dd, J=8.7, 6.7 Hz, 4H), 7.16 (dd, J=7.2, 3.4 Hz, 4H), 3.97 (s, 6H). LCMS: 1.899 min, MS: ES+287.7.

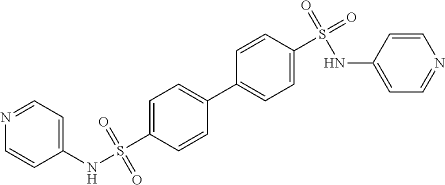 [SMILES: O=S(=O)(Nc1ccncc1)c1ccc(-c2ccc(S(=O)(=O)Nc3ccncc3)cc2)cc1]

### Example 26

N⁴,N^{4′}-di(pyridin-4-yl)-[1,1′-biphenyl]-4,4′-disulfonamide (Example 26) (General procedure G): [1,1′-biphenyl]-4,4′-disulfonyl dichloride (176 mg, 0.5 mmol) and pyridin-4-amine (95 mg, 1 mmol) were mixed in dry DMF (2.0 mL, 0.75 M) then added DIPEA (323 mg, 2.5 mmol) and stirred overnight under argon at RT. The reaction mixture was dissolved in minimum amount of DMSO and purified on a 50 g C18 reversed-phase column (acetonitrile/water). Product was isolated as a TFA salt. (white solid, 0.233 g, 20%). ¹H NMR (400 MHz, DMSO) δ 8.16 (d, J=6.8 Hz, 4H), 8.00-7.90 (m, 5H), 7.86 (d, J=8.5 Hz, 3H), 7.11 (d, J=6.6 Hz, 4H). ¹⁹F NMR (376 MHz, DMSO-D₆) d −73.82. LCMS: 1.764 min, MS: ES+467.1.

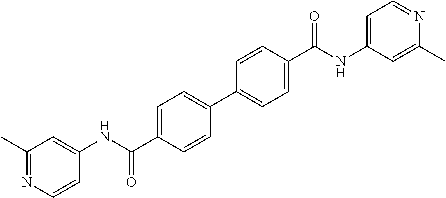

### Example 27

N⁴,N^{4′}-bis(2-methylpyridin-4-yl)-[1,1′-biphenyl]-4,4′-dicarboxamide (Example 27): Prepared from terephthaloyl chloride and 2-methylpyridin-4-amine following the procedure A (orange solid, 0.345 g, 82%.). ¹H NMR (400 MHz, DMSO) δ 10.58 (s, 2H), 8.36 (d, J=5.6 Hz, 2H), 8.11 (d, J=8.4 Hz, 4H), 7.97 (d, J=8.5 Hz, 4H), 7.71 (d, J=1.7 Hz, 2H), 7.63 (dd, J=5.6, 1.8 Hz, 2H), 2.46 (s, 6H). LCMS: 1.906 min, MS: ES+423.2.

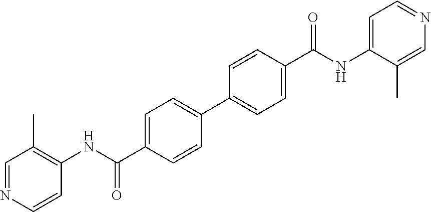 [SMILES: Cc1cnccc1NC(=O)c1ccc(-c2ccc(C(=O)Nc3ccncc3C)cc2)cc1]

### Example 28

N⁴,N^{4′}-bis(3-methylpyridin-4-yl)-[1,1′-biphenyl]-4,4′-dicarboxamide (Example 28): Prepared from terephthaloyl chloride and 3-methylpyridin-4-amine following the procedure A. The reaction mixture was dissolved in minimum amount of DMSO and purified on a 50 g C18 reversed-phase column (acetonitrile/water). Product was isolated as a TFA salt (light orange solid, 0.127 g, 30%.). ¹H NMR (400 MHz, DMSO) δ 10.37 (s, 2H), 8.71 (s, 2H), 8.66 (d, J=6.2 Hz, 2H), 8.35 (d, J=6.2 Hz, 2H), 8.16 (d, J=8.3 Hz, 4H), 8.01 (d, J=8.3 Hz, 4H), 2.48 (s, 6H). ¹⁹F NMR (376 MHz, DMSO-D₆) d −73.82. LCMS: 1.842 min, MS: ES+423.2.

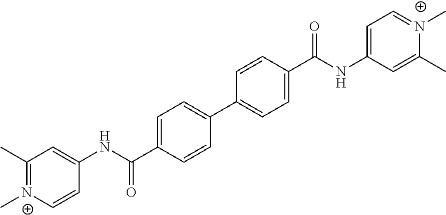

### Example 29

4,4′-(([1,1′-biphenyl]-4,4′-dicarbonyl)bis(azanediyl))bis(1,2-dimethylpyridin-1-ium) (Example 29): Prepared from N⁴,N^{4′}-bis(2-methylpyridin-4-yl)-[1,1′-biphenyl]-4,4′-dicarboxamide (example 27) and methyl p-toluenesulfonate following the procedure D (white solid, 0.025 g, 46%). ¹H NMR (400 MHz, DMSO) δ 11.87 (s, 2H), 8.79 (d, J=7.2 Hz, 2H), 8.41 (s, 2H), 8.28 (d, J=7.7 Hz, 6H), 8.04 (d, J=8.3 Hz, 4H), 4.10 (s, 6H), 2.74 (s, 6H). LCMS: 1.942 min, MS: ES+451.2.

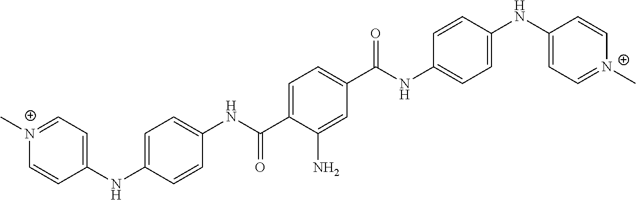

### Example 30

4,4′-((((2-aminoterephthaloyl)bis(azanediyl))bis(4,1-phenylene))bis(azanediyl))bis(1-methylpyridin-1-ium) (Example 30) (procedure H): 4,4′-((((2-nitroterephthaloyl)bis(azanediyl))bis(4,1-phenylene))bis(azanediyl))bis(1-methylpyridin-1-ium) (example 25) (23 mg, 0.034 mmol) and Fe powder (10 mg, 0.17 mmol, 5 equiv.) were mixed in water (0.25 mL) and ethanol (0.5 mL) and stirred for 1 hour at 78° C. The reaction mixture was diluted in additional hot ethanol and the Fe was filtered through a pad of celite and PTFE filter. The solvent was removed under reduced pressure and purified using 50 g C18 reversed-phase column (acetonitrile/water). Product was isolated as a TFA salt. (yellow solid, 0.082 g, 92%). ¹H NMR (400 MHz, DMSO) δ 10.65 (s, 2H), 10.44 (s, 1H), 10.34 (s, 1H), 8.26 (d, J=7.3 Hz, 4H), 7.92 (d, J=8.8 Hz, 2H), 7.87 (d, J=8.8 Hz, 2H), 7.80 (d, J=8.3 Hz, 1H), 7.38-7.29 (m, 5H), 7.15 (dd, J=22.7, 7.5 Hz, 5H), 3.95 (s, 6H), 2.54 (s, 2H). ¹⁹F NMR (376 MHz, DMSO-D₆) d −73.75. LCMS: 1.880 min, MS: ES+272.7.

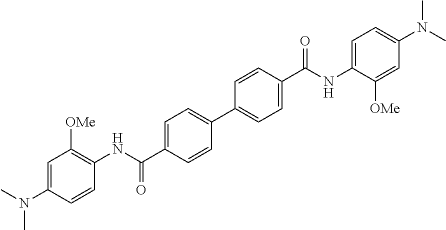

### Example 31

N⁴,N^{4′}-bis(4-(dimethylamino)-2-methoxyphenyl)-[1,1′-biphenyl]-4,4′-dicarboxamide (Example 31): Prepared from terephthaloyl chloride and 3-methoxy-N¹,N¹-dimethylbenzene-1,4-diamine following the procedure A (light green solid, 0.449 g, 83%). ¹H NMR (400 MHz, DMSO) δ 9.34 (s, 2H), 8.07 (d, J=8.1 Hz, 4H), 7.89 (d, J=8.3 Hz, 4H), 7.42 (d, J=8.6 Hz, 2H), 6.41 (d, J=2.4 Hz, 2H), 6.32 (dd, J=8.8, 2.5 Hz, 2H), 3.82 (s, 6H), 2.93 (s, 12H). LCMS: 2.132 min, MS: ES+539.3.

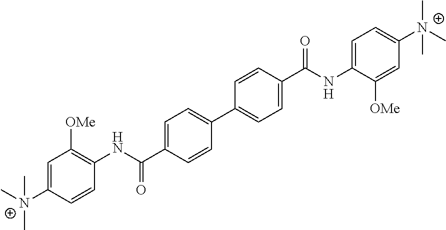

### Example 32

4,4′-(([1,1′-biphenyl]-4,4′-dicarbonyl)bis(azanediyl))bis(3-methoxy-1-methylpyridin-1-ium) (Example 32): Prepared from example 31 and methyl p-toluenesulfonate following the procedure D (white solid, 0.218 g, 92%.). ¹H NMR (400 MHz, DMSO) δ 9.82 (s, 2H), 8.12 (d, J=8.4 Hz, 4H), 8.00 (d, J=9.0 Hz, 2H), 7.95 (d, J=8.4 Hz, 4H), 7.71 (d, J=2.8 Hz, 2H), 7.54 (dd, J=9.0, 2.8 Hz, 2H), 3.99 (s, 6H), 3.67 (s, 18H). LCMS: 2.074 min, MS: ES+284.2.

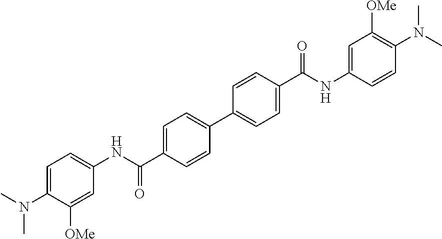

### Example 33

N⁴,N^{4′}-bis(4-(dimethylamino)-3-methoxyphenyl)-[1,1′-biphenyl]-4,4′-dicarboxamide (Example 33): Prepared from terephthaloyl chloride and 2-methoxy-N¹,N¹-dimethylbenzene-1,4-diamine following the procedure A (yellow solid, 0.318 g, 59%). ¹H NMR (400 MHz, DMSO) δ 10.16 (s, 2H), 8.10 (d, J=8.4 Hz, 4H), 7.93 (d, J=8.5 Hz, 4H), 7.48 (d, J=2.2 Hz, 2H), 7.35 (dd, J=8.6, 2.2 Hz, 2H), 6.86 (d, J=8.6 Hz, 2H), 3.80 (s, 6H), 2.67 (s, 12H).

### Example 34. Synthesis of 4,4′-(([1,1′-biphenyl]-4,4′-dicarbonyl)bis(azanediyl))bis(1,2-dimethylpyridin-1-ium) (Example 34)

Prepared from example 28 and methyl p-toluenesulfonate following the procedure D (white solid, 0.114 g, 90%.) ¹H NMR (400 MHz, DMSO) δ 10.78 (s, 2H), 8.91 (s, 2H), 8.78 (d, J=6.7 Hz, 2H), 8.54 (d, J=6.9 Hz, 2H), 8.22 (d, J=8.4 Hz, 4H), 8.02 (d, J=8.5 Hz, 4H), 4.23 (s, 6H), 2.54 (s, 6H). LCMS: 2.461 min, MS: ES+539.3.

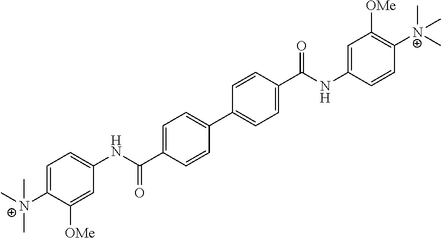

### Example 35

4,4′-(([1,1′-biphenyl]-4,4′-dicarbonyl)bis(azanediyl))bis(2-methoxy-N,N,N-trimethylbenzenaminium) (Example 35): Prepared from example 33 and methyl p-toluenesulfonate following the procedure D (white solid, 0.118 g, 68%.). ¹H NMR (400 MHz, DMSO) δ 10.83 (s, 2H), 8.21 (d, J=8.5 Hz, 4H), 8.02 (d, J=2.2 Hz, 2H), 7.98 (d, J=8.5 Hz, 4H), 7.78 (d, J=9.3 Hz, 2H), 7.69 (dd, J=9.2, 2.1 Hz, 2H), 4.02 (s, 6H), 3.66 (s, 18H). LCMS: 2.138 min, MS: ES+284.2.

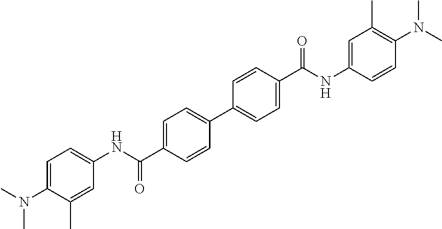

### Example 36

N⁴,N^{4′}-bis(4-(dimethylamino)-3-methylphenyl)-[1,1′-biphenyl]-4,4′-dicarboxamide (Example 36): Prepared from [1,1′-biphenyl]-4,4′-dicarboxylic acid and N¹,N¹,2-trimethylbenzene-1,4-diamine following the procedure C (light green solid, 0.311 g, 62%.). ¹H NMR (400 MHz, DMSO) δ 9.73 (s, 2H), 8.10 (d, J=8.3 Hz, 4H), 7.90 (d, J=8.3 Hz, 4H), 7.11 (d, J=8.6 Hz, 2H), 6.64 (d, J=2.6 Hz, 2H), 6.59 (dd, J=8.7, 2.7 Hz, 2H), 2.90 (s, 12H), 2.19 (s, 6H). LCMS: 1.902 min, MS: ES+507.3.

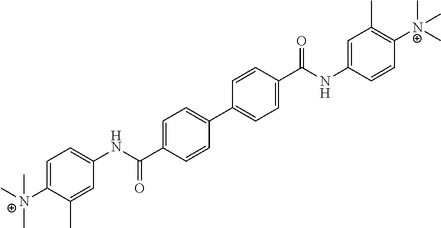

### Example 37

4,4′-(([1,1′-biphenyl]-4,4′-dicarbonyl)bis(azanediyl))bis(N,N,N,2-tetramethylbenzenaminium) (Example 37): Prepared from example 36 and methyl p-toluenesulfonate following the procedure D (white solid, 0.211 g, 89%.). ¹H NMR (400 MHz, DMSO) δ 10.28 (s, 2H), 8.17 (d, J=8.4 Hz, 4H), 7.96 (d, J=8.3 Hz, 6H), 7.83 (dd, J=8.9, 3.1 Hz, 2H), 7.64 (d, J=9.0 Hz, 2H), 3.63 (s, 18H), 2.38 (s, 6H). LCMS: 1.888 min, MS: ES+268.3.

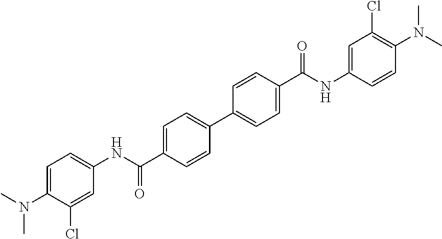

### Example 38

N⁴,N^{4′}-bis(3-chloro-4-(dimethylamino)phenyl)-[1,1′-biphenyl]-4,4′-dicarboxamide (Example 38): Prepared from terephthaloyl chloride and 2-chloro-N¹,N¹-dimethylbenzene-1,4-diamine following the procedure A (grey solid, 0.424 g, 90%.). ¹H NMR (400 MHz, DMSO) δ 10.48 (s, 2H), 8.12 (d, J=8.4 Hz, 4H), 8.03 (d, J=2.3 Hz, 2H), 7.95 (d, J=8.4 Hz, 4H), 7.77 (dd, J=8.8, 2.3 Hz, 2H), 7.38 (d, J=8.8 Hz, 2H), 2.84 (s, 12H). LCMS: 2.188 min, MS: ES+547.2.

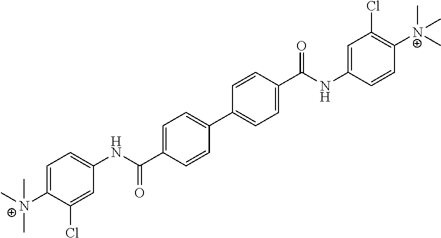

### Example 39

4,4′-(([1,1′-biphenyl]-4,4′-dicarbonyl)bis(azanediyl))bis(2-chloro-N,N,N-trimethylbenzenaminium) (Example 39): Prepared from example 38 above and methyl p-toluenesulfonate following the procedure D (flesh colored solid, 0.157 g, 88%.). ¹H NMR (400 MHz, DMSO) δ 8.26 (d, J=2.3 Hz, 2H), 8.12 (d, J=8.4 Hz, 4H), 8.03 (d, J=9.5 Hz, 2H), 8.01-7.98 (m, 2H), 7.96 (t, J=5.9 Hz, 4H), 3.78 (s, 18H). LCMS: 2.248 min, MS: ES+288.2.

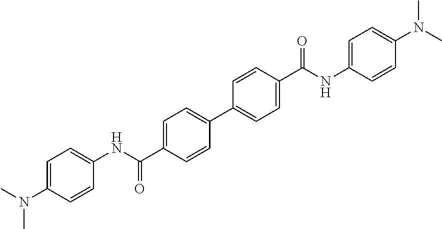

### Example 40

N⁴,N^{4′}-bis(4-(dimethylamino)phenyl)-2-methyl-[1,1′-biphenyl]-4,4′-dicarboxamide (Example 40): Prepared from 2-methyl-[1,1′-biphenyl]-4,4′-dicarboxylic acid and N¹,N¹-dimethylbenzene-1,4-diamine following the procedure C (light green solid, 0.081 g, 82%.). ¹H NMR (400 MHz, DMSO) δ 10.06 (s, 1H), 10.00 (s, 1H), 8.04 (d, J=8.2 Hz, 2H), 7.92 (s, 1H), 7.86 (d, J=7.9 Hz, 1H), 7.60 (d, J=7.3 Hz, 4H), 7.53 (d, J=8.2 Hz, 2H), 7.38 (d, J=7.9 Hz, 1H), 6.74 (d, J=9.0 Hz, 4H), 2.88 (s, 12H), 2.35 (s, 3H). LCMS: 1.990 min, MS: ES+493.3.

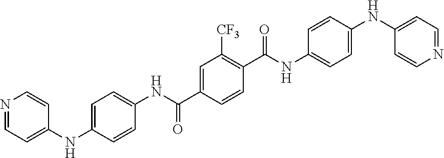 [SMILES: O=C(Nc1ccc(Nc2ccncc2)cc1)c1ccc(C(=O)Nc2ccc(Nc3ccncc3)cc2)c(C(F)(F)F)c1]

### Example 41

N¹,N⁴-bis(4-(pyridin-4-ylamino)phenyl)-2-(trifluoromethyl)terephthalamide (Example 41): Prepared from 2-(trifluoromethyl)terephthaloyl dichloride and N¹-(pyridin-2-yl)benzene-1,4-diamine following the procedure E (tan colored solid, 0.058 g, 21%.). ¹H NMR (400 MHz, DMSO) δ 10.83 (s, 1H), 10.72 (s, 1H), 10.22 (s, 2H), 8.42-8.39 (m, 2H), 8.27-8.24 (m, 4H), 7.92 (dd, J=8.1, 2.6 Hz, 3H), 7.80 (d, J=8.7 Hz, 2H), 7.35 (t, J=7.8 Hz, 4H), 7.05 (d, J=6.9 Hz, 4H). ¹⁹F NMR (376 MHz, DMSO) d −57.99, −73.45. LCMS: 1.942 min, MS: ES+569.3.

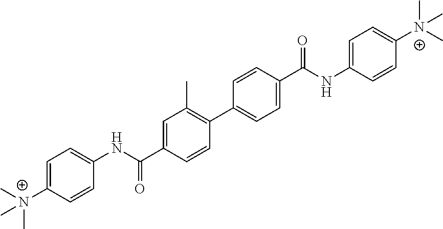

### Example 42

4,4′-((2-methyl-[1,1′-biphenyl]-4,4′-dicarbonyl)bis(azanediyl))bis(N,N,N-trimethylbenzenaminium) (Example 42): Prepared from example 40 and methyl p-toluenesulfonate following the procedure D (white solid, 0.049 g, 82%.). ¹H NMR (400 MHz, DMSO) δ 10.86 (s, 1H), 10.81 (s, 1H), 8.16 (d, J=7.9 Hz, 2H), 8.08 (t, J=8.9 Hz, 5H), 7.99 (d, J=9.4 Hz, 5H), 7.58 (d, J=8.2 Hz, 2H), 7.43 (d, J=7.9 Hz, 1H), 3.63 (s, 18H), 2.37 (s, 3H). LCMS: 2.041 min, MS: ES+261.2.

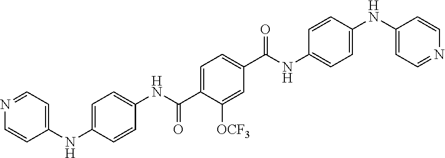 [SMILES: O=C(Nc1ccc(Nc2ccncc2)cc1)c1ccc(C(=O)Nc2ccc(Nc3ccncc3)cc2)c(OC(F)(F)F)c1]

### Example 43

N¹,N⁴-bis(4-(pyridin-4-ylamino)phenyl)-2-(trifluoromethoxy)terephthalamide (Example 43): Prepared from 2-(trifluoromethoxy)terephthaloyl dichloride and N¹-(pyridin-2-yl)benzene-1,4-diamine following the procedure E (tan colored solid, 0.125 g, 43%.). ¹H NMR (400 MHz, DMSO) δ 10.80 (s, 1H), 10.67 (s, 1H), 10.36 (s, 2H), 8.31-8.23 (m, 4H), 8.18 (d, J=8.0 Hz, 1H), 8.06 (s, 1H), 7.97-7.88 (m, 3H), 7.83 (d, J=8.8 Hz, 2H), 7.36 (dd, J=8.7, 5.0 Hz, 4H), 7.07 (d, J=7.1 Hz, 4H). ¹⁹F NMR (376 MHz, DMSO) d −56.43, −73.49. LCMS: 2.029 min, MS: ES+585.2.

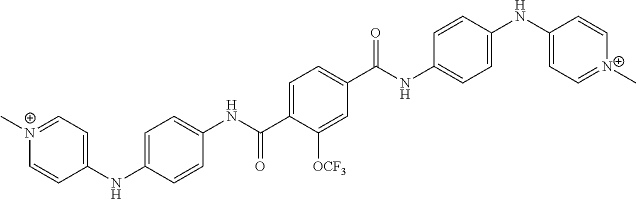

### Example 44

4,4′-((((2-(trifluoromethoxy)terephthaloyl)bis(azanediyl))bis(4,1-phenylene))bis(azanediyl))bis(1-methylpyridin-1-ium) (Example 44): Prepared from example 43 and methyl p-toluenesulfonate following the procedure D (tan colored solid, 0.037 g, 60%). ¹H NMR (400 MHz, DMSO) δ 11.02 (s, 1H), 10.98 (s, 1H), 10.88 (s, 1H), 10.86 (s, 1H), 8.26 (td, J=7.9, 2.4 Hz, 5H), 8.10 (s, 1H), 7.97 (d, J=8.9 Hz, 2H), 7.91 (d, J=8.0 Hz, 1H), 7.84 (d, J=8.8 Hz, 2H), 7.36 (dd, J=8.8, 4.9 Hz, 4H), 7.19 (d, J=6.1 Hz, 4H), 3.96 (s, 6H). LCMS: 2.127 min, MS: ES+307.2.

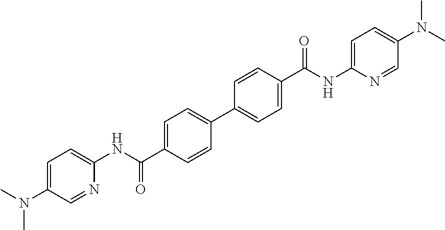

### Example 45

N⁴,N^{4′}-bis(6-(dimethylamino)pyridin-3-yl)-[1,1′-biphenyl]-4,4′-dicarboxamide (Example 45): Prepared from biphenyl-4,4′-dicarboxylic acid and N²,N²-dimethylpyridine-2,5-diamine following the procedure C (green solid, 0.070 g, 58%). ¹H NMR (400 MHz, DMSO) δ 10.56 (s, 2H), 8.15 (d, J=8.4 Hz, 4H), 8.00 (d, J=9.0 Hz, 2H), 7.90 (t, J=6.6 Hz, 6H), 7.26 (dd, J=9.1, 3.1 Hz, 2H), 2.93 (s, 12H). LCMS: 2.187 min, MS: ES+481.3.

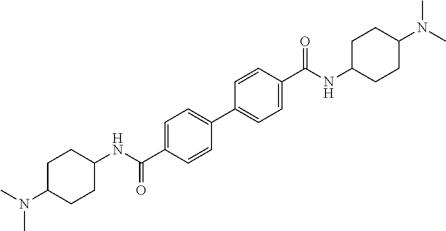

### Example 46

N⁴,N^{4′}-bis(4-(dimethylamino)cyclohexyl)-[1,1′-biphenyl]-4,4′-dicarboxamide (Example 46): Prepared from biphenyl-4,4′-dicarboxylic acid and N¹,N¹-dimethylcyclohexane-1,4-diamine following the procedure C (white solid, 0.060 g, 51%). ¹H NMR (400 MHz, 2:1 ratio of diastereomers, DMSO) δ 8.31 (d, J=7.7 Hz, 2H), 8.22 (d, J=7.1 Hz, 2H), 7.96 (t, J=5.9 Hz, 8H), 7.87-7.77 (m, 8H), 3.96 (s, 4H), 2.90 (s, 4H), 2.39 (s, 12H), 2.29 (s, 12H), 1.93 (s, 8H), 1.83 (s, 8H), 1.55 (s, 8H), 1.39 (s, 8H). LCMS: 1.751 min, MS: ES+491.4.

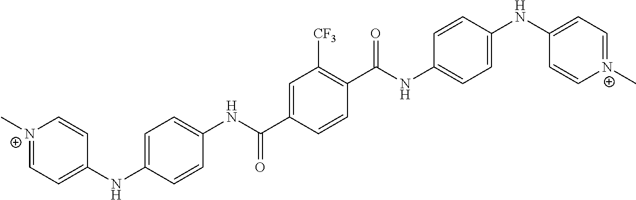

### Example 47

4,4′-((((2-(trifluoromethyl)terephthaloyl)bis(azanediyl))bis(4,1-phenylene))bis(azanediyl))bis(1-methylpyridin-1-ium)) (Example 47): Prepared from example 41 and methyl p-toluenesulfonate following the procedure D (tan colored solid, 0.024 g, 36%). ¹H NMR (400 MHz, DMSO) δ 8.36 (s, 1H), 8.33 (d, J=8.0 Hz, 1H), 8.17 (dd, J=7.6, 2.3 Hz, 4H), 7.85 (dd, J=10.4, 8.5 Hz, 3H), 7.77 (d, J=8.8 Hz, 2H), 7.38-7.29 (m, 4H), 7.07 (dd, J=7.6, 2.7 Hz, 4H), 3.90 (s, 6H). LCMS: 2.156 min, MS: ES+299.2.

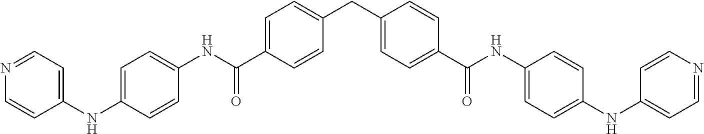 [SMILES: O=C(Nc1ccc(Nc2ccncc2)cc1)c1ccc(Cc2ccc(C(=O)Nc3ccc(Nc4ccncc4)cc3)cc2)cc1]

### Example 48

N¹,N⁴-bis(4-(pyridin-4-ylamino)phenyl)-2-(trifluoromethoxy)terephthalamide (Example 48): Prepared from 4,4′-methylenedibenzoyl chloride and N¹-(pyridin-2-yl)benzene-1,4-diamine following the procedure E (tan colored solid, 0.368 g, 89%). ¹H NMR (400 MHz, DMSO) δ 10.48 (s, 2H), 10.36 (s, 2H), 8.26 (d, J=7.2 Hz, 4H), 7.91 (t, J=8.6 Hz, 8H), 7.44 (d, J=8.3 Hz, 4H), 7.33 (d, J=8.9 Hz, 4H), 7.06 (d, J=7.3 Hz, 4H), 4.14 (s, 2H). ¹⁹F NMR (376 MHz, DMSO) δ −73.51. LCMS: 2.178 min, MS: ES+591.3.

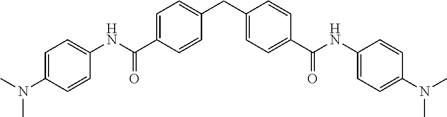

### Example 49

4,4′-methylenebis(N-(4-(dimethylamino)phenyl)benzamide) (Example 49): Prepared from 4,4′-methylenedibenzoic acid and N¹,N¹-dimethylbenzene-1,4-diamine following the procedure C (black solid, 0.369 g, 98%). ¹H NMR (400 MHz, DMSO) δ 10.12 (s, 2H), 7.89 (d, J=8.2 Hz, 4H), 7.70 (d, J=8.7 Hz, 4H), 7.41 (d, J=8.2 Hz, 4H), 7.11 (s, 4H), 4.12 (s, 2H) 2.99 (s, 12H). ¹⁹F NMR (376 MHz, DMSO) d-74.57. LCMS: 2.041 min, MS: ES+493.3.

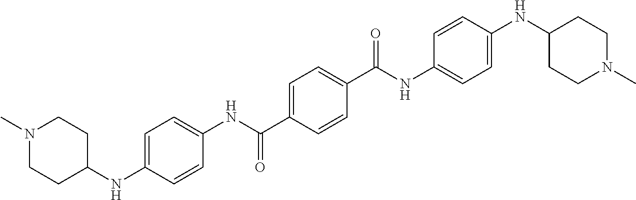

### Example 50

N¹,N⁴-bis(4-((1-methylpiperidin-4-yl)amino)phenyl)terephthalamide (Example 50): Prepared from terephthaloyl chloride and N¹-(1-methylpiperidin-4-yl)benzene-1,4-diamine following the procedure E (brown solid, 0.120 g, 78%). ¹H NMR (400 MHz, DMSO) δ 10.12 (s, 2H), 9.65 (s, 2H), 8.04 (s, 4H), 7.52 (d, J=8.5 Hz, 4H), 6.68 (d, J=8.2 Hz, 4H), 3.46 (t, J=13.1 Hz, 5H), 3.31 (d, J=13.5 Hz, 1H), 3.21 (dd, J=19.7, 9.3 Hz, 1H), 3.05 (dd, J=22.9, 11.2 Hz, 3H), 2.78 (s, 3H), 2.77 (s, 3H), 2.13 (d, J=13.5 Hz, 3H), 1.94 (dt, J=14.8, 11.5 Hz, 2H), 1.58 (dd, J=24.1, 11.3 Hz, 3H). ¹⁹F NMR (376 MHz, DMSO) δ −74.38. LCMS: 2.043 min, MS: ES+541.3.

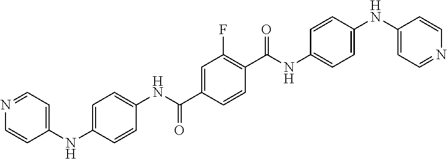 [SMILES: O=C(Nc1ccc(Nc2ccncc2)cc1)c1ccc(C(=O)Nc2ccc(Nc3ccncc3)cc2)c(F)c1]

### Example 51

2-fluoro-N¹,N⁴-bis(4-(pyridin-4-ylamino)phenyl)terephthalamide (Example 51): Prepared from 2-fluoroterephthaloyl dichloride and N¹-(pyridin-4-yl)benzene-1,4-diamine following the procedure E (tan colored solid, 0.061 g, 16%). ¹H NMR (400 MHz, DMSO) δ 10.77 (s, 1H), 10.65 (s, 1H), 10.49 (s, 2H), 8.27 (d, J=6.5 Hz, 4H), 8.01-7.90 (m, 4H), 7.86 (d, J=8.7 Hz, 3H), 7.36 (d, J=6.8 Hz, 4H), 7.08 (d, J=5.8 Hz, 4H). ¹⁹F NMR (376 MHz, DMSO) d −73.47, −114.09. LCMS: 1.763 min, MS: ES+519.3.

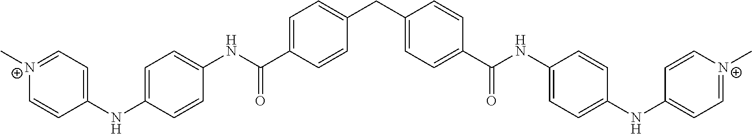

### Example 52

4,4′-((((4,4′-methylenebis(benzoyl))bis(azanediyl))bis(4,1-phenylene))bis(azanediyl))bis(1-methylpyridin-1-ium) (Example 52): Prepared from example 48 and methyl p-toluenesulfonate following the procedure D (light yellow solid, 0.016 g, 28%). ¹H NMR (400 MHz, DMSO) δ 10.97 (s, 2H), 10.46 (s, 2H), 8.24 (d, J=7.2 Hz, 4H), 7.94 (dd, J=12.1, 8.6 Hz, 8H), 7.43 (d, J=8.2 Hz, 4H), 7.31 (d, J=8.7 Hz, 4H), 7.15 (s, 4H), 4.14 (s, 2H), 3.94 (s, 6H). LCMS: 2.082 min, MS: ES+310.2.

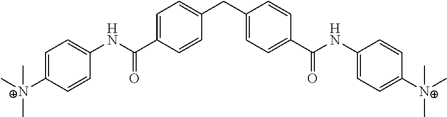

### Example 53

4,4′-((4,4′-methylenebis(benzoyl))bis(azanediyl))bis(N,N,N-trimethylbenzenaminium)) (Example 53): Prepared from example 49 and methyl p-toluenesulfonate following the procedure D (tan colored solid, 0.063 g, 98%). ¹H NMR (400 MHz, DMSO) δ 10.71 (s, 2H), 8.04 (d, J=9.4 Hz, 4H), 7.97 (dd, J=16.5, 8.8 Hz, 8H), 7.44 (d, J=8.2 Hz, 4H), 4.14 (s, 2H), 3.60 (s, 18H). LCMS: 2.126 min, MS: ES+261.2.

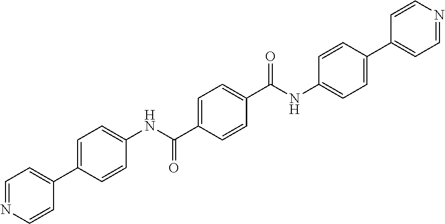 [SMILES: O=C(Nc1ccc(-c2ccncc2)cc1)c1ccc(C(=O)Nc2ccc(-c3ccncc3)cc2)cc1]

### Example 54

N¹,N⁴-bis(4-(pyridin-4-yl)phenyl)terephthalamide (Example 54): Prepared from terephthaloyl chloride and 4-(pyridin-4-yl)aniline following the procedure A (light yellow solid, 0.200 g, 85%). ¹H NMR (400 MHz, DMSO) δ 10.61 (s, 2H), 8.62 (d, J=5.2 Hz, 4H), 8.14 (s, 4H), 7.97 (t, J=9.0 Hz, 4H), 7.88 (d, J=8.2 Hz, 4H), 7.74 (d, J=5.1 Hz, 4H). LCMS: 1.767 min, MS: ES+471.2.

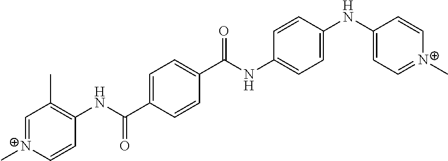

### Example 55

### 1,3-dimethyl-4-(4-((4-((1-methylpyridin-1-ium-4-yl)amino)phenyl)carbamoyl)benzamido)pyridin-1-ium (Example 55)

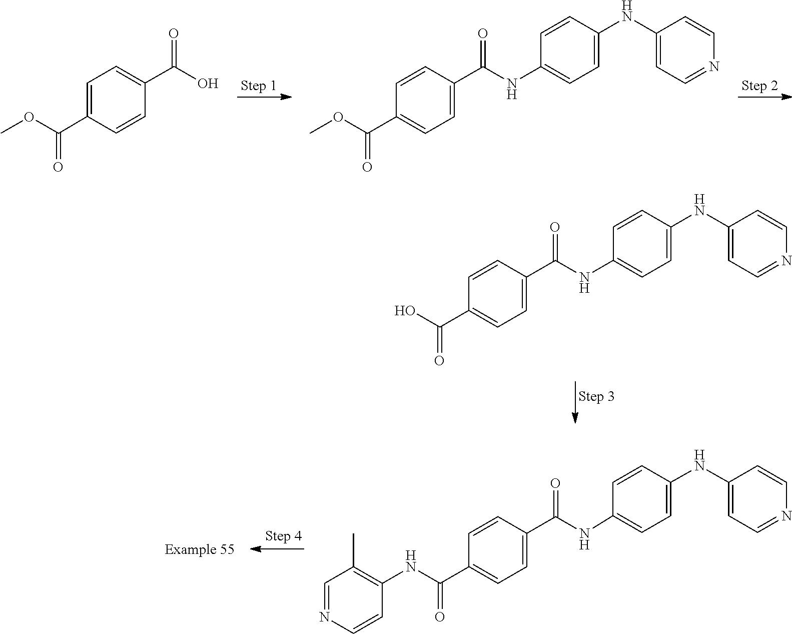 [SMILES: COC(=O)c1ccc(C(=O)O)cc1 ; COC(=O)c1ccc(C(=O)Nc2ccc(Nc3ccncc3)cc2)cc1 ; O=C(O)c1ccc(C(=O)Nc2ccc(Nc3ccncc3)cc2)cc1 ; Cc1cnccc1NC(=O)c1ccc(C(=O)Nc2ccc(Nc3ccncc3)cc2)cc1]

Step 1: Synthesis of methyl 4-((4-(pyridin-4-ylamino)phenyl)carbamoyl)benzoate: 4-(Methoxycarbonyl)benzoic acid (0.180 g, 1.00 mmol) and 1-N-(Pyridin-4-yl)benzene-1,4-diamine (0.204 g, 1.10 mmol) were combined in ethyl acetate (0.5 mL) and to this mixture was added pyridine (0.242 mL, 3.00 mmol) followed by T3P (1.19 mL, 2.00 mmol, 50% wt in ethyl acetate). After stirring at room temperature overnight the reaction was cooled in an ice bath and quenched by the addition of 0.5 M HCl. The precipitate that formed was collected by filtration and dried under vacuum for several days to give the desired compound (0.364 g, >100%) as a dark purple solid. ¹H-NMR (400 MHz, DMSO-d₆) δ ppm 10.70 (s, 2H), 8.30 (d, J=7.0 Hz, 2H), 8.15 (m, 4H), 8.10 (s, 1H), 7.96 (d, J=8.8 Hz, 2H), 7.40 (d, J=8.8 Hz, 2H), 7.14 (d, J=6.7 Hz, 2H). 3.95 (s. 3H). LCMS: 1.91 min, MS: ES+348.2

Step 2: Synthesis of 4-((4-(pyridin-4-ylamino)phenyl)carbamoyl)benzoic acid: Crude ester from above (0.300 g, 0.864 mmol) was suspended in tetrahydrofuran (4.0 mL) and treated with a solution of LiOH (0.062 g, 2.59 mmol) in water (2.0 mL). The resulting mixture was stirred at room temperature for 3 hours at which time the reaction was concentrated to remove the organics. The aqueous reaction mixture was treated with a sat'd solution of NH₄Cl and the resulting mixture was stirred at room temperature for 30 minutes. The precipitate that formed was collected by filtration washing with water and then hexanes. The solids were dried under vacuum overnight to give the product (0.176 g, 61%) as a pale purple solid. ¹H-NMR (400 MHz, DMSO-d₆) δ ppm 10.43 (s, 1H), 8.84 (s, 1H), 8.20 (d, J=6.2 Hz, 2H), 8.12-8.06 (m, 4H), 7.80 (d, J=8.8 Hz, 2H), 7.24 (d, J=8.8 Hz, 2H), 6.90 (d, J=6.3 Hz, 2H). LCMS: 1.67 min, MS: ES+334.1

Step 3: Synthesis of N¹-(3-methylpyridin-4-yl)-N⁴-(4-(pyridin-4-ylamino)phenyl)terephthalamide: The crude acid from above (0.057 g, 0.171 mmol) and 4-amino-3-methylpridine (0.020, 0.188 mmol) were combined in DMF (0.5 mL) and to the mixture was added diisopropylethylamine (0.089 mL, 0.513 mmol) followed by PyBroP (0.159 g, 0.342 mmol). After stirring at room temperature for 4 hours the reaction was quenched by the addition of water. The pH of the reaction mixture was adjusted to approximately 9-10 with 1.0 N NaOH. The precipitate that formed was collected by filtration. The collected solids were dissolved in methanol, absorbed onto silica gel and purified by silica gel chromatography (10% methanol in ethyl acetate and then with a 2.5%, followed by a 5.0% and finally a 10% solution of (7N NH₃ in methanol) in ethyl acetate) to give the product (0.023 g, 18%) as a yellow solid. LCMS: 1.64 min, MS: ES+424.2

Step 4: Synthesis of 1,3-dimethyl-4-(4-((4-((1-methylpyridin-1-ium-4-yl)amino)phenyl)carbamoyl)benzamido)pyridin-1-ium: The crude product from above (0.023 g 0.054 mmol) was added to a solution of iodomethane (0.020 g, 0.141 mmol) in DMF (1.0 mL) in a microwave vial that was sealed and stirred at room temperature for 48 hours. Water was added to the reaction mixture which was stirred at room temperature for several hours. The precipitate that developed was collected by filtration washing with water and then hexanes to give (0.013 g, 34%) as a bright yellow solid. ¹H-NMR (400 MHz, DMSO-d₆) δ ppm 11.0 (br s, 1H), 10.8 (m, 2H), 8.93 (br s, 1H), 8.80 (br d, J=6.1 Hz, 1H), 8.31 (d, J=7.2 Hz, 2H), 8.27-8.22 (m, 4H), 8.02 (d, J=8.7 Hz, 2H), 7.39 (d, J=6.0 Hz, 2H), 7.21 (br d, J=6.0 Hz, 2H), 4.27 (s, 3H), 4.00 (s, 3H), 2.54 (d, J=6.3 Hz, 3H). LCMS: 1.64 min, MS: ES+452.2

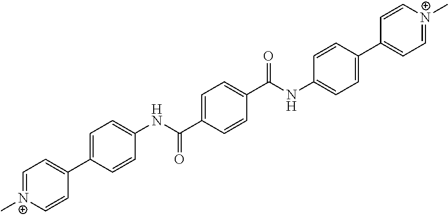

### Example 56

4,4′-((terephthaloylbis(azanediyl))bis(4,1-phenylene))bis(1-methylpyridin-1-ium)) (Example 56): Prepared from example 54 and methyl p-toluenesulfonate according to procedure D (white solid, 0.053 g, 84%). ¹H NMR (400 MHz, DMSO) δ 8.80 (d, J=6.7 Hz, 4H), 8.37 (d, J=6.7 Hz, 4H), 8.09 (d, J=8.0 Hz, 8H), 8.03 (d, J=8.8 Hz, 4H), 4.26 (s, 6H). LCMS: 1.768 min, MS: ES+250.2.

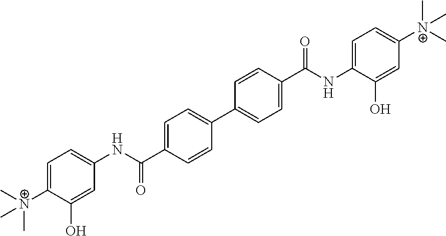

### Example 57

4,4′-(([1,1′-biphenyl]-4,4′-dicarbonyl)bis(azanediyl))bis(3-hydroxy-N,N,N-trimethylbenzenaminium) (Example 57) (General Procedure I): Example 32 (50 mg, 0.09 mmol) was dissolved in dry DCM (2 ml) and cooled the reaction mixture to 0° C. and added boron tribromide (BBr₃) (0.2 ml) and allowed the reaction to come to room temperature and stirred overnight. The solvent was removed, and the crude product was dissolved in minimum amount of DMSO and purified on a 50 g C18 reversed-phase column (acetonitrile/water). Product was isolated as a TFA salt (white solid, 0.052 g, 75%). ¹H NMR (400 MHz, DMSO) δ 10.70 (s, 2H), 9.91 (s, 2H), 8.15 (d, J=8.2 Hz, 4H), 7.96 (d, J=8.3 Hz, 4H), 7.90 (d, J=8.8 Hz, 2H), 7.46 (d, J=11.0 Hz, 4H), 3.59 (s, 18H). ¹⁹F NMR (376 MHz, DMSO) δ −73.45. LCMS: 1.883 min, MS: ES+270.2.

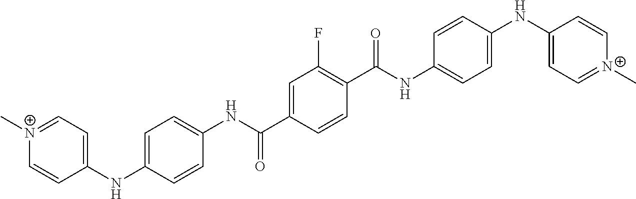

### Example 58

4,4′-((((2-fluoroterephthaloyl)bis(azanediyl))bis(4,1-phenylene))bis(azanediyl))bis(1-methylpyridin-1-ium) (Example 58): Prepared from example 51 and methyl p-toluenesulfonate according to procedure D (yellow solid, 0.049 g, 79%). ¹H NMR (400 MHz, DMSO) δ 11.12 (s, 1H), 11.10 (s, 1H), 10.83 (s, 1H), 10.81 (s, 1H), 8.28 (d, J=7.1 Hz, 4H), 8.01 (dt, J=22.0, 12.2 Hz, 5H), 7.88 (d, J=8.9 Hz, 2H), 7.36 (d, J=7.9 Hz, 4H), 7.20 (s, 4H), 3.96 (s, 6H). LCMS: 1.892 min, MS: ES+274.1.

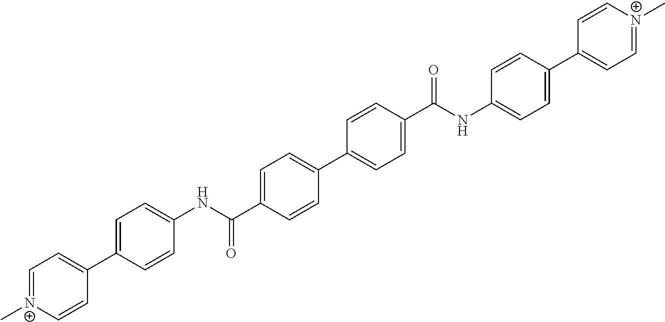

### Example 59

4,4′-((([1,1′-biphenyl]-4,4′-dicarbonyl)bis(azanediyl))bis(4,1-phenylene))bis(1-methylpyridin-1-ium) (Example 59): Prepared from N⁴,N^{4′}-bis(4-(pyridin-4-yl)phenyl)-[1,1′-biphenyl]-4,4′-dicarboxamide and methyl p-toluenesulfonate following the procedure D (white solid, 0.025 g, 43%). ¹H NMR (400 MHz, DMSO) δ 8.86 (d, J=6.4 Hz, 4H), 8.41 (d, J=6.6 Hz, 4H), 8.10 (dd, J=17.8, 9.3 Hz, 12H), 7.95 (d, J=8.2 Hz, 4H), 4.28 (s, 6H). LCMS: 2.032 min, MS: ES+288.2.

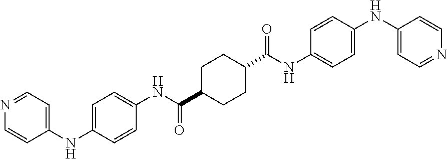 [SMILES: O=C(Nc1ccc(Nc2ccncc2)cc1)[C@H]1CC[C@H](C(=O)Nc2ccc(Nc3ccncc3)cc2)CC1]

### Example 60

(E)-N¹,N⁴-bis(4-(pyridin-4-ylamino)phenyl)cyclohexane-1,4-dicarboxamide (Example 60): Prepared from (E)-cyclohexane-1,4-dicarbonyl dichloride and N¹-(pyridin-4-yl)benzene-1,4-diamine following the procedure E (tan colored solid, 0.077 g, 41%). ¹H NMR (400 MHz, DMSO) δ 10.32 (s, 2H), 10.09 (s, 2H), 8.23 (d, J=6.2 Hz, 4H), 7.73 (d, J=7.7 Hz, 4H), 7.25 (d, J=7.6 Hz, 4H), 7.01 (d, J=6.1 Hz, 4H), 2.39 (s, 2H), 1.93 (d, J=8.5 Hz, 4H), 1.58-1.43 (m, 4H). ¹⁹F NMR (376 MHz, DMSO) δ −73.48. LCMS: 1.719 min, MS: ES+507.2.

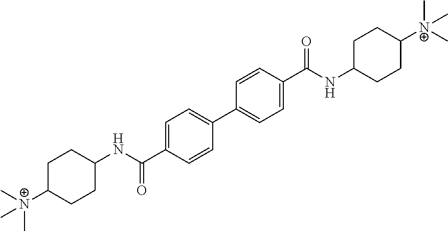

### Example 61

4,4′-(([1,1′-biphenyl]-4,4′-dicarbonyl)bis(azanediyl))bis(N,N,N-trimethylcyclohexan-1-aminium) (Example 61): Prepared from example 46 and methyl p-toluenesulfonate following the procedure D (white solid, 0.049 g, 82%). 1H NMR (400 MHz, 2:1 ratio of diastereomers, DMSO) δ 8.53 (s, 1H), 8.51 (s, 1H), 8.37 (s, 1H), 8.35 (s, 1H), 8.00 (d, J=6.6 Hz, 8H), 7.82 (d, J=7.9 Hz, 8H), 4.14 (dd, J=10.1, 5.1 Hz, 2H), 3.89-3.79 (m, 2H), 3.06 (s, 36H), 2.68 (s, 1H), 2.33 (s, 1H), 2.19 (d, J=10.4 Hz, 10H), 2.08-1.88 (m, 10H), 1.66 (dd, J=24.0, 12.2 Hz, 8H), 1.50 (dd, J=24.7, 12.0 Hz, 6H). LCMS: 1.624 min, MS: ES+260.3.

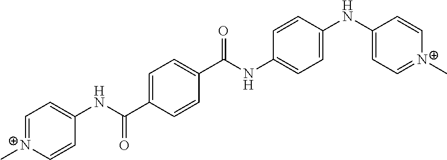

### Example 62

### 1-methyl-4-(4-((4-((1-methylpyridin-1-ium-4-yl)amino)phenyl)carbamoyl)benzamido)pyridin-1-ium (Example 62)

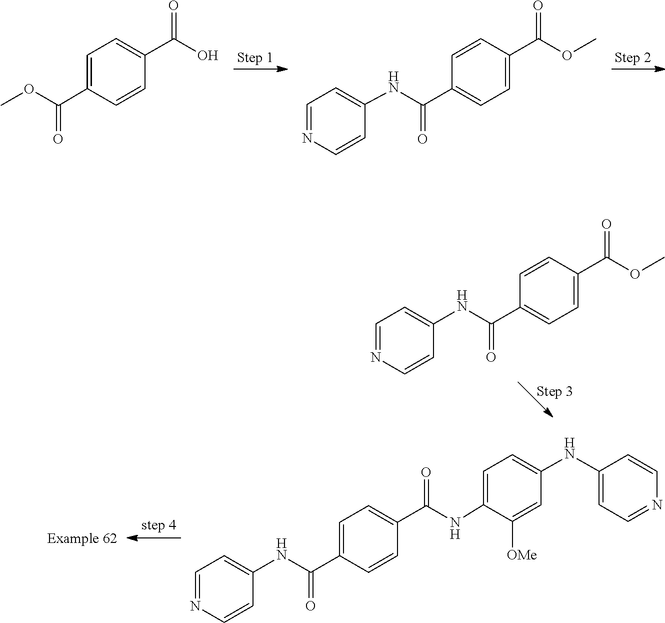 [SMILES: COC(=O)c1ccc(C(=O)O)cc1 ; COC(=O)c1ccc(C(=O)Nc2ccncc2)cc1 ; COC(=O)c1ccc(C(=O)Nc2ccncc2)cc1 ; COc1cc(Nc2ccncc2)ccc1NC(=O)c1ccc(C(=O)Nc2ccncc2)cc1]

Step 1: Synthesis of methyl 4-(pyridin-4-ylcarbamoyl)benzoate: 4-(Methoxycarbonyl) benzoic acid (2.00 g, 11.10 mmol) suspended in dichloromethane (25 mL) and DMF (0.1 mL) was cooled in an ice-bath. To the ice-cold suspension was slowly added oxalyl chloride (11.10 mL, 2.0 M in dichloromethane, 22.20 mmol). The resulting mixture was allowed to slowly warm to room temperature overnight. The reaction was concentrated and then dissolved in tetrahydrofuran (150 mL). To this was added a mixture of 4-aminopyridine (1.11 g, 11.77 mmol) and triethylamine (8.66 mL, 62.16 mmol) in tetrahydrofuran (100 mL). After stirring at room temperature overnight the precipitate that formed was filtered off. The filtrate was partially concentrated, and water was added to the mixture. The precipitate that formed was collected by filtration washing with water and then diethyl ether to give the product (2.49 g, 88%) as an off-white solid. ¹H-NMR (400 MHz, DMSO-d₆) δ ppm 10.81 (s, 1H), 8.55-8.53 (m, 2H), 8.17-8.11 (m, 4H), 7.83-7.81 (m, 2H), 3.94 (s, 3H). LCMS: 1.74 min, MS: ES+257.1

Step 2: Synthesis of 4-(pyridin-4-ylcarbamoyl)benzoic acid: To a suspension of of the above ester (2.40 g, 9.37 mmol) in tetrahydrofuran (20 mL) was added a solution of LiOH (0.673 g, 28.10 mmol) in water (10 mL) at room temperature. The resulting mixture was stirred at room temperature for 2 hours and then concentrated to remove the organics. The aqueous reaction mixture was made acidic (pH 5-6) with 6N HCl and a small amount of concentrated HCl. The precipitate that formed was collected by filtration washing with water and then diethyl ether to give the product (2.57 g, >100%) as white powder. ¹H-NMR (400 MHz, DMSO-d₆) δ ppm 13.40 (br s, 1H), 11.81 (s, 1H), 8.80 (d, J=7.1 Hz, 2H), 8.37 (s, J=6.9 Hz, 2H), 8.22-8.15 (m, 4H), 8.08 (s, 1H). LCMS: 1.47 min, MS: ES+243.1

Step 3: Synthesis of N¹-(pyridin-4-yl)-N⁴-(4-(pyridin-4-ylamino)phenyl)terephthalamide: To a suspension of above acid (0.125 g, 0.516 mmol) in dichloroethane (1.0 mL) was added diisopropylethylamine (0.461 mL, 2.58 mmol) followed by bis(tetramethylene)fluoroformamidinium hexafluorophosphate (0.196 g, 0.619 mmol). The resulting mixture was stirred at room temperature for 1.5 hours at which time 1-N-(Pyridin-4-yl) benzene-1,4-diamine (0.105 g, 0.568 mmol) was added. After 2 hours additional bis(tetramethylene)fluoroformamidinium hexafluorophosphate (0.082 g, 0.258 mmol) was added and stirring was continued at room temperature. After 45 minutes additional bis(tetramethylene)fluoroformamidinium hexafluorophosphate (0.065 g, 0.206 mmol) was added and stirring was continued at room temperature overnight. The reaction mixture was diluted with dichloromethane and the precipitate that formed was collected by filtration. The resulting residue was subjected twice to preparative HPLC (RP18, acetonitrile/water w/0/1% TFA). The fractions containing the desired compound were partially concentrated to remove the acetonitrile. The aqueous layer was treated with 1.0 N NaOH to adjust pH>10 and then the aqueous layer was concentrated. A small amount of water was added to the residue and the precipitate was collected by filtration washing with a minimum amount of water and then diethyl ether to give the product (0.020 g, 10%) as a bright yellow solid. ¹H-NMR (400 MHz, DMSO-d₆) δ ppm 10.79 (s, 1H), 10.53 (s, 1H), 9.59 (s, 1H), 8.54 (d, J=5.7 Hz, 2H), 8.25 (d, J=6.2 Hz, 2H), 8.16 (m, 4H), 7.90-7.85 (m, 4H), 7.32 (d, J=8.7 Hz, 2H), 7.00 (d, J=6.2 Hz, 2H). LCMS: 1.68 min, MS: ES+410.1

Step 4: Synthesis of 1-methyl-4-(4-((4-((1-methylpyridin-1-ium-4-yl)amino)phenyl)carbamoyl)benzamido)pyridin-1-ium: To a solution of above pyridyl compound (0.020 g 0.049 mmol) in DMF (0.5 mL) was added to a solution of iodomethane (0.122 mL g, 1.0 M in DMF, 0.122 mmol) in a microwave vial that was sealed and stirred at room temperature for 48 hours. The solvent was removed by azeotrope with toluene to give the desired product (0.31 g, 91%) as a yellow/orange solid. ¹H-NMR (400 MHz, DMSO-d₆) δ ppm 11.71 (br s, 1H), 10.64 (s, 1H), 10.47 (s, 1H), 8.83-8.79 (br m, 2H), 8.31-8.29 (m, 4H), 8.24-8.18 (m, 4H), 7.96 (d, J=8.8 Hz, 2H), 7.39 (d, J=8.8 Hz, 2H), 7.23 (d, J=7.5 Hz, 2H) 4.26 (s, 3H), 4.02 (s, 3H). LCMS: 1.70 min, MS: ES+219.6

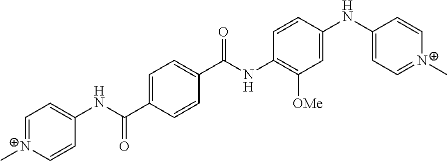

### Example 63

### 4-(4-((2-methoxy-4-((1-methylpyridin-1-ium-4-yl)amino)phenyl)carbamoyl)benzamido)-1-methylpyridin-1-ium (Example 63)

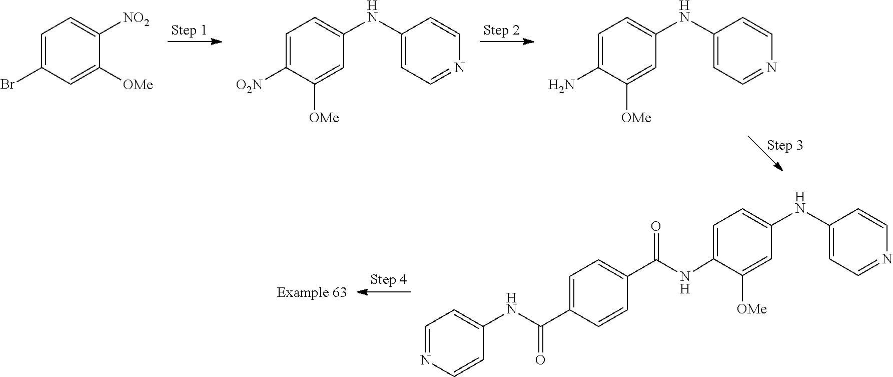 [SMILES: COc1cc(Br)ccc1[N+](=O)[O-] ; COc1cc(Nc2ccncc2)ccc1[N+](=O)[O-] ; COc1cc(Nc2ccncc2)ccc1N ; COc1cc(Nc2ccncc2)ccc1NC(=O)c1ccc(C(=O)Nc2ccncc2)cc1]

Step 1: Synthesis of N-(2-methoxy-4-nitrophenyl)pyridin-4-amine. 5-Bromo-2-nitro-methoxybenzene (0.464 g, 2.00 mmol), 4-aminopyridine (0.226 g, 2.40 mmol) and Cs₂CO₃ (1.63 g, 5.00 mmol) were suspended in dioxane (6.0 mL) and purged with argon. To the suspension was added BINAP (0.125 g, 0.200 mmol) and Pd₂(dba)₃-CHCl₃ (0.104 g, 0.100 mmol). The resulting mixture was heated to 80° C. for 20 h. After cooling to room temperature, the reaction was filtered through a pad of Celite® washing with methanol. The filtrate was concentrated and then water was added. The resulting mixture was sonicated for several minutes before the solids were collected by filtration washing with water and then dichloromethane. The nitro product was isolated (0.366 g, 75%) as a brick orange solid. ¹H-NMR (400 MHz, DMSO-d₆) δ ppm 8.39-8.37 (dd, J=1.5, 4.8 Hz, 2H), 7.98 (d, J=9.0 Hz, 1H), 7.17-7.15 (dd, J=1.5, 4.8 Hz, 2H), 6.90 (d, J=1.9 Hz, 1H), 6.85-6.82 (dd, J=1.7, 4.8 Hz, 1H), 3.94 (s, 3H). LCMS: 1.70 min, MS: ES+246.2.

Step 2: Synthesis of 3-methoxy-N¹-(pyridin-4-yl)benzene-1,4-diamine. To a suspension of the above nitro compound (0.150 g, 0.612 mmol) and ammonium formate (0.154 g, 2.45 mmol) in ethanol (4.0 mL) was added 10% Pd/C (15 mg) and the resulting mixture was heated to reflux for 1 hour. After cooling to room temperature, the reaction mixture was filtered through a pad of Celite washing with methanol. The filtrate was concentrated to give the desired compound (0.131 g, 99%) as a brown oily residue. LCMS: 1.68 min, MS: ES+254.1. LCMS: 0.295 min, MS: ES+216.1

Step 3: Synthesis of N¹-(2-methoxy-4-(pyridin-4-ylamino)phenyl)-N⁴-(pyridin-4-yl)terephthalamide The above amine was reacted with 4-(pyridin-4-ylcarbamoyl)benzoic acid according to step 3 for the procedure outlined for example 62 to give the desired product (0.031 g, 15%) as a yellow/green solid. LCMS: 1.71 min, MS: ES+440.1

Step 4: Synthesis of 4-(4-((2-methoxy-4-((1-methylpyridin-1-ium-4-yl)amino)phenyl)carbamoyl)benzamido)-1-methylpyridin-1-ium. The title compound was prepared according to step 4 of example 62. The product was isolated (0.030 g, 61%) as a bright yellow solid. ¹H-NMR (400 MHz, DMSO-d₆) δ ppm 11.70 (s, 1H), 10.52 (s, 1H), 9.83 (s, 1H), 8.80 (br m, 2H), 8.33-8.31 (m 4H), 8.20 (m, 4H), 7.88 (d, J=8.4 Hz, 1H), 7.20 (d, J=7.3 Hz, 2H), 7.09 (d, J=2.1 Hz, 1H), 7.03-7.00 (dd, J=2.1, 8.4 Hz, 1H), 4.24 (s, 3H), 4.02 (s, 3H). 3.91 (s, 3H). LCMS: 1.73 min, MS: ES+468.2.

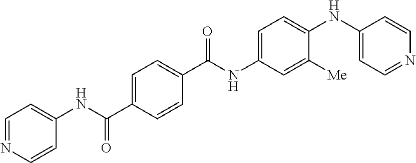 [SMILES: Cc1cc(NC(=O)c2ccc(C(=O)Nc3ccncc3)cc2)ccc1Nc1ccncc1]

### Example 64

### N¹-(3-methyl-4-(pyridin-4-ylamino)phenyl)-N⁴-(pyridin-4-yl)terephthalamide (Example 64)

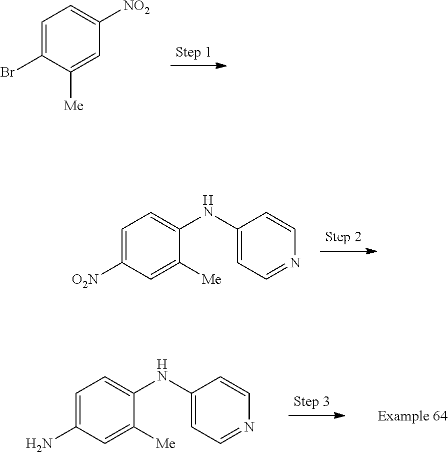 [SMILES: Cc1cc([N+](=O)[O-])ccc1Br ; Cc1cc([N+](=O)[O-])ccc1Nc1ccncc1 ; Cc1cc(N)ccc1Nc1ccncc1]

Step 1: Synthesis of N-(2-methyl-4-nitrophenyl)pyridin-4-amine. The desired aniline was prepared from 2-methyl-4-nitro-bromobenzene and 4-aminopyridine according to the procedure outlined in step for example 62. The nitro product was isolated (0.267 g, 56%) as a mustard colored solid. ¹H NMR (400 MHz, DMSO-d₆) δ ppm 8.67 (s, 1H), 8.39 (br s, 2H), 8.19 (d, J=2.2 Hz, 1H), 8.09-8.06 (dd, J=2.5, 8.9 Hz, 1H), 7.51 (d, J=8.9 Hz, 1H), 7.10 (br d, J=4.4 Hz, 2H), 2.39 (s, 3H). LCMS: 1.79 min, MS: ES+230.2.

Step 2: Synthesis of N-(2-methyl-4-aminophenyl)pyridin-4-amine. The nitro compound was reduced according to step 2 for the procedure outlined in example 62. The product was isolated (0.128 g, 98%) as a tan foam. LCMS: 0.277 min, MS: ES+200.1

Step 3: Synthesis of N¹-(3-methyl-4-(pyridin-4-ylamino)phenyl)-N⁴-(pyridin-4-yl)terephthalamide. The desired product was prepared according to the procedure outlined in step 3 of example 62. The desired product was isolated (0.044 g, 21%) as a bright yellow solid. ¹H NMR (400 MHz, DMSO-d₆) δ ppm 10.77 (s, 1H), 10.42 (s, 1H), 8.54 (dd, J=1.5, 4.8 Hz, 2H), 8.24 (s, 1H), 8.17-8.11 (m, 6H), 7.86-7.84 (dd, J=1.5, 4.8 Hz, 2H), 7.78 (d, J=2.2 Hz, 1H), 7.71-7.68 (dd, J=2.2, 8.6 Hz, 1H), 7.25 (d, J=8.6 Hz, 1H), 6.62-6.60 (dd, J=1.5, 4.8 Hz, 2H), 2.23 (s, 3H). LCMS: 1.71 min, MS: ES+424.1

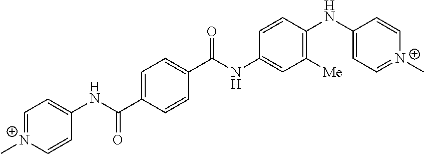

### Example 65

### 1-methyl-4-(4-((3-methyl-4-((1-methylpyridin-1-ium-4-yl)amino)phenyl)carbamoyl)benzamido)pyridin-1-ium (Example 65)

The desired product was prepared according to the procedure in step 4 outlined in example 63. The product was isolated (0.048 g, 96%) as a yellow solid. ¹H-NMR (400 MHz, DMSO-d₆) δ ppm 11.70 (s, 1H), 10.59 (s, 1H), 10.15 (s, 1H), 8.79 (br m, 2H), 8.34-8.29 (br m, 2H), 8.28-8.25 (m 2H), 8.24-8.18 (m, 4H), 7.89 (d, J=2.2 Hz, 1H), 7.84-7.81 (dd, J=2.2, 8.5 Hz, 1H), 7.32 (d, J=8.5 Hz, 2H), 4.24 (s, 3H), 3.98 (s, 3H). 2.24 (s, 3H). LCMS: 1.75 min, MS: ES+452.2.

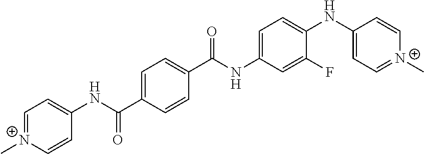

### Example 66

### 1-methyl-4-(4-((3-fluoro-4-((1-methylpyridin-1-ium-4-yl)amino)phenyl)carbamoyl)benzamido)pyridin-1-ium (Example 66)

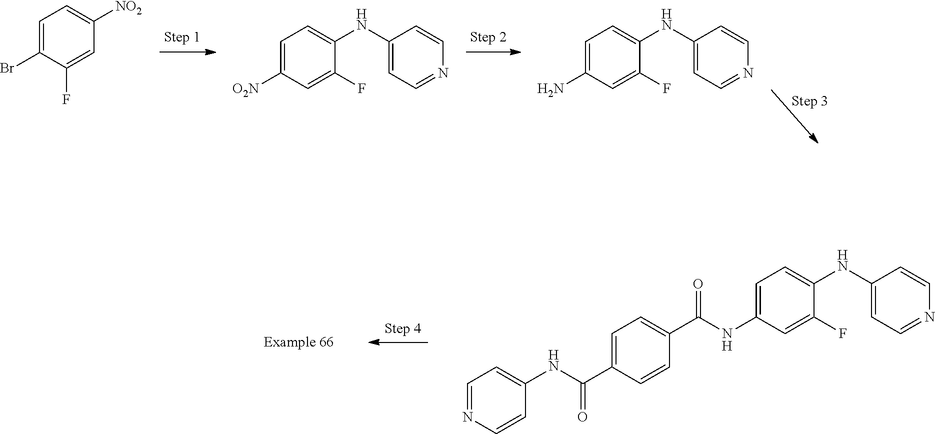 [SMILES: O=[N+]([O-])c1ccc(Br)c(F)c1 ; O=[N+]([O-])c1ccc(Nc2ccncc2)c(F)c1 ; Nc1ccc(Nc2ccncc2)c(F)c1 ; O=C(Nc1ccncc1)c1ccc(C(=O)Nc2ccc(Nc3ccncc3)c(F)c2)cc1]

Step 1: Synthesis of N-(2-fluoro-4-nitrophenyl)pyridin-4-amine. The aniline was prepared from 2-fluoro-4-nitro-bromobenzene and 4-aminopyridine according to step 1 outlined in example 62. The nitro product was isolated (0.238 g, 51%) as a brick orange solid. ¹H-NMR (400 MHz, DMSO-d₆) δ ppm 9.39 (br s, 1H), 8.40 (d, J=5.7 Hz, 2H), 8.19-8.16 (dd, J=2.0, 9.5 Hz, 1H), 8.06 (d, J=9.1 Hz, 1H), 7.58 (t, J=Hz, 8.8 Hz, 1H), 7.15 (d, J=5.7 Hz, 2H). LCMS: 1.57 min, MS: ES+234.1.

Step 2: Synthesis of N-(2-methyl-4-aminophenyl)pyridin-4-amine. The above nitro compound was reduced according to step 2 for the procedure outlined in example 62. The product was isolated (0.120 g, 92%) as a tan solid. ¹H-NMR (400 MHz, DMSO-d₆) δ ppm 8.08-8.06 (m, 3H), 6.97 (t, J=8.8 Hz, 1H), 6.50-6.48 (m, 2H), 6.45-6.42 (m, 1H), 5.41 (s, 2H). LCMS: 0.411 min, MS: ES+204.1

Step 3: Synthesis of N¹-(3-fluoro-4-(pyridin-4-ylamino)phenyl)-N⁴-(pyridin-4-yl)terephthalamide. To a suspension of the above aniline (0.100 g, 0.492 mmol) and 4-(pyridin-4-ylcarbamoyl)benzoic acid (0.155 g, 0.640 mmol) in DMF (1.5 mL) was added diisopropylethylamine (0.439 mL, 2.46 mmol). The resulting mixture was stirred at room temperature until all the solids dissolved at which time the solution of T3P (0.574 mL, 50% in DMF, 0.984 mmol) was slowly added. After 5 minutes the reaction mixture was partially concentrated by azeotrope with toluene. After cooling to room temperature water was added to the reaction mixture and the solids were collected by filtration. The crude compound was subjected to preparative HPLC (RP18, acetonitrile/water w/0/1% TFA). The fractions containing the desired compound were partially concentrated to remove the acetonitrile. The aqueous layer was treated with 1.0 N NaOH to adjust pH≥10 and then the aqueous layer was concentrated. A small amount of water was added to the residue and the precipitate was collected by filtration washing with a minimum amount of water and then diethyl ether to give the product (0.070 g, 33%) as a bright yellow solid. ¹H-NMR (400 MHz, DMSO-d₆) δ ppm 10.78 (br s, 1H), 10.65 (br s, 1H), 8.59 (s, 1H), 8.54 (dd, J=1.4, 4.9 Hz, 2H), 8.20 (d, J=6.2 Hz, 2H), 8.16 (m, 4H), 7.95-7.91 (dd, J=2.1, 13.1 Hz, 1H) 7.32 (dd, J=1.4, 4.9 Hz, 2H), 7.65-7.62 (dd, J=1.7, 8.6 Hz, 2H), 7.42 (t, J=8.9 Hz, 1H), 6.74 (d, J=5.5 Hz, 2H). LCMS: 1.71 min, MS: ES+428.1

Step 4: 1-methyl-4-(4-((3-fluoro-4-((1-methylpyridin-1-ium-4-yl)amino)phenyl)carbamoyl)benzamido)pyridin-1-ium. To a solution of the above compound (0.020 g 0.047 mmol) in DMF (0.5 mL) in a microwave vial was added to a solution of iodomethane (0.117 mL, 1M in DMF, 0.117 mmol). The microwave vial was sealed and stirred at room temperature for 24 hours. The reaction mixture was concentrated by azeotrope with toluene. The residue was subjected to preparative HPLC (RP18, acetonitrile/water w/0/1% TFA) to give the product (0.030 g, 94%) as a yellow solid. ¹H-NMR (400 MHz, DMSO-d₆) δ ppm 11.92 (s, 1H), 10.95 (s, 1H), 10.53 (s, 1H), 8.89 (d, J=7.4 Hz, 2H), 8.43 (d, J=7.4 Hz, 2H), 8.39 (d, J=7.5 Hz, 2H), 8.32-8.26 (m, 4H), 8.13-8.09 (dd, J=2.2, 13.0 Hz, 1H), 7.83-7.81 (dd, J=1.7, 8.6 Hz, 1H), 7.57 (t, J=8.9 Hz, 1H), 7.10 (br s, 2H), 4.31 (s, 3H), 4.07 (s, 3H). LCMS: 1.74 min, MS: ES+456.2.

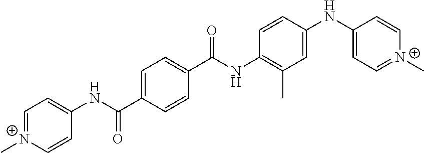

### Example 67

### 1-methyl-4-(4-((2-methyl-4-((1-methylpyridin-1-ium-4-yl)amino)phenyl)carbamoyl)benzamido)pyridin-1-ium (Example 67)

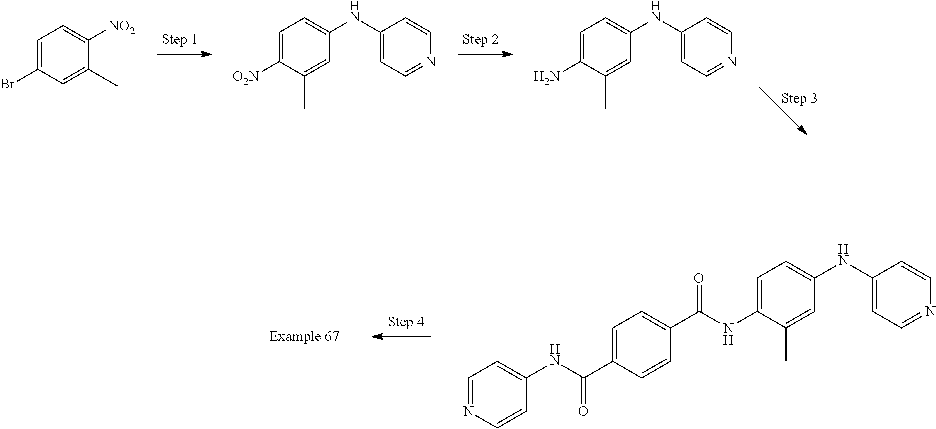 [SMILES: Cc1cc(Br)ccc1[N+](=O)[O-] ; Cc1cc(Nc2ccncc2)ccc1[N+](=O)[O-] ; Cc1cc(Nc2ccncc2)ccc1N ; Cc1cc(Nc2ccncc2)ccc1NC(=O)c1ccc(C(=O)Nc2ccncc2)cc1]

Step 1: Synthesis of N-(3-methyl-4-nitrophenyl)pyridin-4-amine The aniline was prepared from 3-methyl-4-nitro-bromobenzene and 4-aminopyridine according to step 1 for the procedure outlined for example 62. The nitro product was isolated (0.386 g, 60%) as a bright yellow solid. ¹H-NMR (400 MHz, DMSO-d₆) δ ppm 9.60 (s, 1H), 8.39 (d, J=5.9 Hz, 2H), 8.10 (d, J=9.7 Hz, 1H), 7.22 (m, 2H), 7.17 (d, J=6.0 Hz, 2H). LCMS: 1.76 min, MS: ES+230.1.

Step 2: Synthesis of N-(3-methyl-4-aminophenyl)pyridin-4-amine The reduced product was prepared according to step 2 for the procedure outlined for example 62. The product was isolated (0.120 g, 92%) as a brick orange solid. ¹H-NMR (400 MHz, DMSO-d₆) δ ppm 8.29 (br s, 1H), 8.07-8.06 (dd, J=1.4, 4.9 Hz, 2H), 6.82-6.77 (m, 2H), 6.66-6.63 (m, 3H), 2.09 (s, 3H). LCMS: 0.353 min, MS: ES+200.1

Step 3: Synthesis of N¹-(2-methyl-4-(pyridin-4-ylamino)phenyl)-N⁴-(pyridin-4-yl)terephthalamide The coupled product was obtained according to step 3 for the procedure outlined for example 66 to give the product (0.023 g, 11%) as a pale-yellow solid. LCMS: 1.65 min, MS: ES+424.1

Step 4: 1-methyl-4-(4-((2-methyl-4-((1-methylpyridin-1-ium-4-yl)amino)phenyl)carbamoyl)benzamido)pyridin-1-ium was prepared as outlined in example 66 the product was isolated (0.039 g, >100%) as a yellow solid. ¹H-NMR (400 MHz, DMSO-d₆) δ ppm 11.88 (s, 1H), 10.64 (s, 1H), 10.25 (s, 1H), 8.83 (d, J=7.3 Hz, 2H), 8.40 (d, J=7.2 Hz, 2H), 8.32 (d, J=7.5 Hz, 2H), 8.27-8.21 (m, 4H), 7.53 (d, J=8.4 Hz, 1H), 7.31 (d, J=2.3 Hz, 1H), 7.27-7.24 (dd, J=2.3, 8.4 Hz, 1H), 7.19 (d, J=7.5 Hz, 2H), 4.26 (s, 3H), 4.01 (s, 3H). 2.34 (s, 3H). LCMS: 1.74 min, MS: ES+456.2.

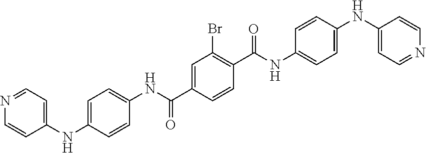 [SMILES: O=C(Nc1ccc(Nc2ccncc2)cc1)c1ccc(C(=O)Nc2ccc(Nc3ccncc3)cc2)c(Br)c1]

### Example 68

2-bromo-N¹,N⁴-bis(4-(pyridin-4-ylamino)phenyl)terephthalamide (Example 68): Prepared from 2-bromoterephthaloyl dichloride and N¹-(pyridin-4-yl)benzene-1,4-diamine following the procedure E (tan colored solid, 0.186 g, 54%). ¹H NMR (400 MHz, DMSO) δ 10.77 (s, 1H), 10.62 (s, 1H), 10.40 (s, 2H), 8.33 (d, J=1.2 Hz, 1H), 8.26 (dd, J=7.0, 2.8 Hz, 4H), 8.11 (dd, J=7.9, 1.3 Hz, 1H), 7.92 (d, J=8.8 Hz, 2H), 7.84 (d, J=8.8 Hz, 2H), 7.75 (d, J=7.9 Hz, 1H), 7.35 (dd, J=8.9, 2.2 Hz, 4H), 7.07 (d, J=6.4 Hz, 4H). ¹⁹F NMR (376 MHz, DMSO) δ −73.47. LCMS: 1.979 min, MS: ES+579.1.

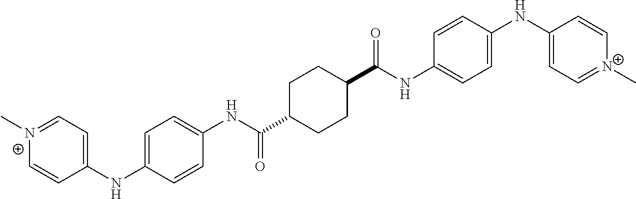

### Example 69

4,4′-((((E)-cyclohexanedicarbonylbis(azanediyl))bis(4,1-phenylene))bis(azanediyl))bis(1-methylpyridin-1-ium) (Example 69): Prepared from (E)-N¹,N⁴-bis(4-(pyridin-4-ylamino)phenyl)cyclohexane-1,4-dicarboxamide (example 60) and methyl p-toluenesulfonate following the procedure D (brown solid, 0.046 g, 69%). ¹H NMR (400 MHz, DMSO) δ 10.26 (s, 2H), 8.24 (d, J=7.5 Hz, 4H), 7.76 (d, J=8.9 Hz, 4H), 7.25 (d, J=8.8 Hz, 4H), 7.12 (s, 4H), 3.93 (s, 6H), 2.43 (dd, J=7.8, 4.0 Hz, 2H), 1.93 (d, J=8.0 Hz, 5H), 1.50 (t, J=10.2 Hz, 5H). LCMS: 1.778 min, MS: ES+268.2.

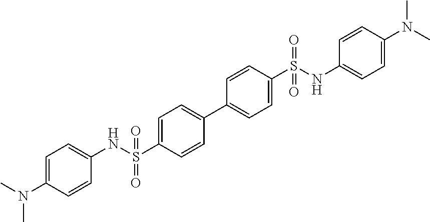

### Example 70

N⁴, N^{4′}-bis(4-(dimethylamino)phenyl)-[1,1′-biphenyl]-4,4′-disulfonamide (Example 70): Prepared from [1,1′-biphenyl]-4,4′-disulfonyl dichloride and N¹,N¹-dimethylbenzene-1,4-diamine following the procedure G (grey solid, 0.645 g, 97%). ¹H NMR (400 MHz, DMSO) δ 9.89 (s, 2H), 7.88 (d, J=8.6 Hz, 4H), 7.77 (d, J=8.5 Hz, 4H), 6.94 (d, J=8.9 Hz, 4H), 6.72 (d, J=7.4 Hz, 4H), 2.84 (s, 12H). ¹⁹F NMR (376 MHz, DMSO) δ −74.66. LCMS: 1.967 min, MS: ES+551.2.

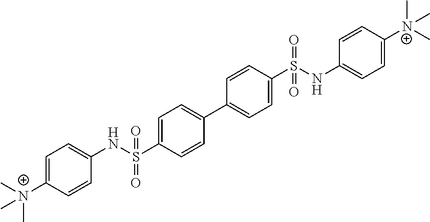

### Example 71

4,4′-(([1,1′-biphenyl]-4,4′-disulfonyl)bis(azanediyl))bis(1-methylpyridin-1-ium) (Example 71): Prepared from N⁴, N^{4′}-bis(4-(dimethylamino)phenyl)-[1,1′-biphenyl]-4,4′-disulfonamide and methyl p-toluenesulfonate following the procedure D (white solid, 0.042 g, 66%). ¹H NMR (400 MHz, DMSO) δ 7.92 (q, J=8.7 Hz, 8H), 7.79 (d, J=9.4 Hz, 4H), 7.28 (d, J=9.3 Hz, 4H), 3.47 (s, 18H). LCMS: 1.967 min, MS: ES+290.2.

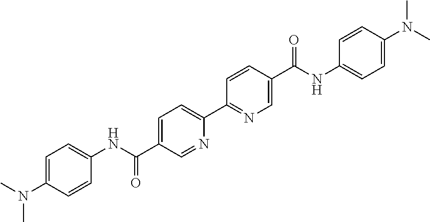

### Example 72

N⁵,N^{5′}-bis(4-(dimethylamino)phenyl)-[2,2′-bipyridine]-5,5′-dicarboxamide (Example 72): General Procedure J [2,2′-bipyridine]-5,5′-dicarboxylic acid (100 mg, 0.4 mmol) and N¹,N¹-dimethylbenzene-1,4-diamine (166 mg, 1.2 mmol) were mixed in dry DMF (0.7 mL) then added DIPEA (520 mg, 4.0 mmol) then added T3P (propanephosphonic acid anhydride, 50% solution in DMF) (510 mg, 1.0 mL, 1.6 mmol) under argon and stirred at RT for overnight. Ether was added to the reaction mixture and filtered to collect the pure product (green solid, 0.146 g, 76%). ¹H NMR (400 MHz, DMSO) δ 10.28 (s, 2H), 9.23 (s, 2H), 8.58 (d, J=8.3 Hz, 2H), 8.48 (dd, J=8.4, 1.9 Hz, 2H), 7.61 (d, J=8.9 Hz, 4H), 6.76 (d, J=9.0 Hz, 4H), 2.89 (s, 12H). LCMS: 1.714 min, MS: ES+481.2.

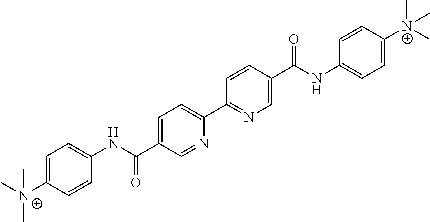

### Example 73

4,4′-(([2,2′-bipyridine]-5,5′-dicarbonyl)bis(azanediyl))bis(N,N,N-trimethylbenzenaminium) (Example 73): Prepared from N⁵, N^{5′}-bis(4-(dimethylamino)phenyl)-[2,2′-bipyridine]-5,5′-dicarboxamide and methyl p-toluenesulfonate following the procedure D (white solid, 0.037 g, 61%). ¹H NMR (400 MHz, DMSO) δ 10.94 (s, 2H), 9.30 (d, J=1.7 Hz, 2H), 8.64 (d, J=8.3 Hz, 2H), 8.57 (dd, J=8.3, 2.2 Hz, 2H), 8.07-7.96 (m, 8H), 3.62 (s, 18H). LCMS: 1.681 min, MS: ES+255.2.

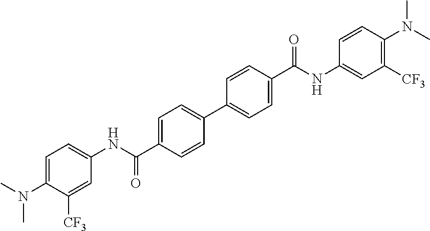

### Example 74

N⁴,N^{4′}-bis(4-(dimethylamino)-3-(trifluoromethyl)phenyl)-[1,1′-biphenyl]-4,4′-dicarboxamide (Example 74): Prepared from [1,1′-biphenyl]-4,4′-dicarboxylic acid and N¹, N¹-dimethyl-2-(trifluoromethyl)benzene-1,4-diamine following the procedure J (light yellow solid, 0.050 g, 30%). ¹H NMR (400 MHz, DMSO) δ 10.51 (s, 2H), 8.18 (d, J=2.5 Hz, 2H), 8.12 (d, J=8.5 Hz, 4H), 8.07 (dd, J=8.8, 2.4 Hz, 2H), 7.96 (d, J=8.5 Hz, 4H), 7.58 (d, J=8.9 Hz, 2H), 2.65 (s, 12H). ¹⁹F NMR (376 MHz, DMSO) δ −58.89, −74.53. LCMS: 3.201 min, MS: ES+615.2.

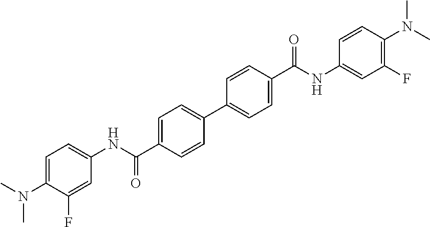

### Example 75

N⁴,N^{4′}-bis(4-(dimethylamino)-3-fluorophenyl)-[1,1′-biphenyl]-4,4′-dicarboxamide (Example 75): Prepared from [1,1′-biphenyl]-4,4′-dicarboxylic acid and 2-fluoro-N¹,N¹-dimethylbenzene-1,4-diamine following the procedure J (light yellow solid, 0.065 g, 63%). ¹H NMR (400 MHz, DMSO) δ 10.30 (s, 2H), 8.08 (d, J=8.4 Hz, 4H), 7.94 (d, J=8.5 Hz, 4H), 7.70 (dd, J=15.4, 2.3 Hz, 2H), 7.48 (dd, J=8.8, 1.7 Hz, 2H), 7.03-6.94 (m, 2H), 2.75 (s, 12H). ¹⁹F NMR (376 MHz, DMSO) d −121.35. LCMS: 2.072 min, MS: ES+515.2.

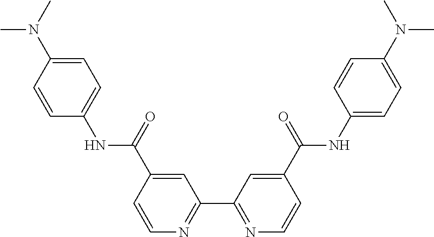

### Example 76

N⁴,N^{4′}-bis(4-(dimethylamino)phenyl)-[2,2′-bipyridine]-4,4′-dicarboxamide (Example 76): Prepared from [2,2′-bipyridine]-4,4′-dicarboxylic acid and N¹,N¹-dimethylbenzene-1,4-diamine following the procedure J (light green solid, 0.091 g, 95%). ¹H NMR (400 MHz, DMSO) δ 10.45 (s, 2H), 8.93 (d, J=5.0 Hz, 2H), 8.90 (s, 2H), 7.97 (dd, J=5.0, 1.5 Hz, 2H), 7.62 (d, J=9.0 Hz, 4H), 6.76 (d, J=9.1 Hz, 4H), 2.89 (s, 12H). LCMS: 1.714 min, MS: ES+481.2.

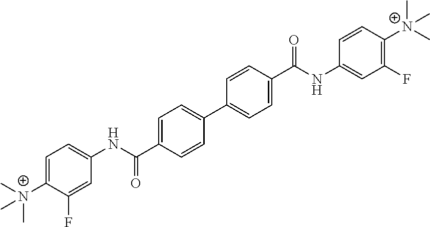

### Example 77

Synthesis of 2-fluoro-4-(4′-{[3-fluoro-4-(trimethylazaniumyl)phenyl]carbamoyl}-[1,1′-biphenyl]-4-amido)-N,N,N-trimethylanilinium (Example 77): Prepared from N⁴,N^{4′}-bis(4-(dimethylamino)-3-fluorophenyl)-[1,1′-biphenyl]-4,4′-dicarboxamide (SLUPP-2140) and methyl p-toluenesulfonate following the procedure D (white solid, 0.026 g, 44%). ¹H NMR (400 MHz, DMSO) δ 11.09 (s, 2H), 8.22 (d, J=8.3 Hz, 5H), 8.18 (d, J=2.0 Hz, 1H), 8.01-7.95 (m, 5H), 7.95-7.85 (m, 3H), 3.70 (s, 18H). LCMS: 1.948 min, MS: ES+272.2.

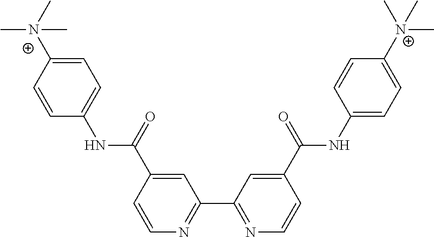

### Example 78

4,4′-(([2,2′-bipyridine]-4,4′-dicarbonyl)bis(azanediyl))bis(N,N,N-trimethylbenzenaminium) (Example 78): Prepared from example 76 and methyl p-toluenesulfonate following the procedure D (tan colored solid, 0.027 g, 49%). ¹H NMR (400 MHz, DMSO) δ 11.24 (s, 2H), 9.00-8.94 (m, 4H), 8.13 (dd, J=5.0, 1.6 Hz, 2H), 8.09 (d, J=9.5 Hz, 4H), 8.02 (d, J=9.6 Hz, 4H), 3.63 (s, 18H). LCMS: 1.650 min, MS: ES+255.2.

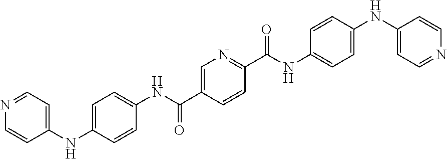 [SMILES: O=C(Nc1ccc(Nc2ccncc2)cc1)c1ccc(C(=O)Nc2ccc(Nc3ccncc3)cc2)nc1]

### Example 79

Synthesis of N²,N⁵-bis(4-(pyridin-4-ylamino)phenyl)pyridine-2,5-dicarboxamide (Example 79): Prepared from pyridine-2,5-dicarbonyl dichloride and N¹-(pyridin-4-yl)benzene-1,4-diamine following the procedure E (brown solid, 0.078 g, 36%). ¹H NMR (400 MHz, DMSO) δ 10.95 (s, 1H), 10.84 (s, 1H), 10.39 (s, 2H), 9.24 (d, J=1.8 Hz, 1H), 8.60 (dd, J=8.2, 2.0 Hz, 1H), 8.33 (d, J=8.2 Hz, 1H), 8.26 (dd, J=7.1, 1.7 Hz, 4H), 8.07 (d, J=8.8 Hz, 2H), 7.92 (d, J=8.8 Hz, 2H), 7.37 (dd, J=8.8, 4.4 Hz, 4H), 7.08 (d, J=7.1 Hz, 4H). ¹⁹F NMR (376 MHz, DMSO) δ −73.46. LCMS: 1.826 min, MS: ES+502.1.

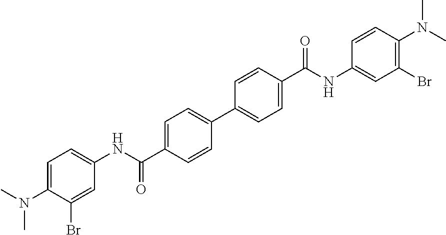

### Example 80

Synthesis of N⁴,N^{4′}-bis(3-bromo-4-(dimethylamino)phenyl)-[1,1′-biphenyl]-4,4′-dicarboxamide (Example 80): Prepared from [1,1′-biphenyl]-4,4′-dicarboxylic acid and 2-bromo-N¹,N¹-dimethylbenzene-1,4-diamine following procedure J (light brown solid, 0.137 g, 46%). ¹H NMR (400 MHz, DMSO) δ 10.34 (s, 2H), 8.13 (d, J=2.4 Hz, 2H), 8.09 (d, J=8.4 Hz, 4H), 7.94 (d, J=8.4 Hz, 4H), 7.76 (dd, J=8.7, 2.4 Hz, 2H), 7.22 (d, J=8.8 Hz, 2H), 2.71 (s, 12H). ¹⁹F NMR (376 MHz, DMSO) d −74.31. LCMS: 2.268 min, MS: ES+637.0.

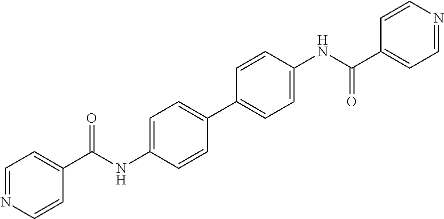 [SMILES: O=C(Nc1ccc(-c2ccc(NC(=O)c3ccncc3)cc2)cc1)c1ccncc1]

### Example 81

Synthesis of N,N′-([1,1′-biphenyl]-4,4′-diyl)diisonicotinamide (Example 81): Prepared from isonicotinoyl chloride and benzidine following the procedure A (light brown solid, 0.052 g, 88%). ¹H NMR (400 MHz, DMSO) δ 10.58 (s, 2H), 8.80 (dd, J=4.4, 1.6 Hz, 4H), 7.88 (dd, J=5.5, 3.9 Hz, 8H), 7.72 (d, J=8.7 Hz, 4H). LCMS: 1.800 min, MS: ES+395.1.

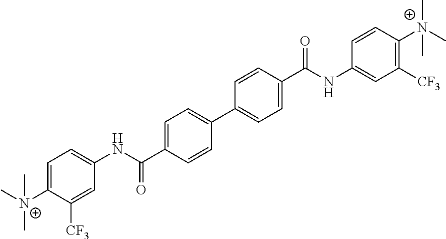

### Example 82

4,4′-(([1,1′-biphenyl]-4,4′-dicarbonyl)bis(azanediyl))bis(N,N,N-trimethyl-2-(trifluoromethyl)benzenaminium) (Example 82): Prepared from example 74 and methyl p-toluenesulfonate following procedure D (light green solid, 0.016 g, 26%). ¹H NMR (400 MHz, DMSO) δ 8.51 (d, J=2.5 Hz, 2H), 8.25 (dd, J=9.3, 2.5 Hz, 2H), 8.15 (d, J=9.5 Hz, 2H), 8.07 (d, J=8.4 Hz, 4H), 7.92 (d, J=8.5 Hz, 4H), 3.71 (s, 18H). LCMS: 2.149 min, MS: ES+322.1.

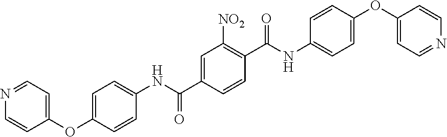 [SMILES: O=C(Nc1ccc(Oc2ccncc2)cc1)c1ccc(C(=O)Nc2ccc(Oc3ccncc3)cc2)c([N+](=O)[O-])c1]

### Example 83

Synthesis of 2-nitro-N¹,N⁴-bis(4-(pyridin-4-yloxy)phenyl)terephthalamide (Example 83): Prepared from 2-nitroterephthaloyl dichloride and N¹-(pyridin-4-yl)benzene-1,4-diamine following the procedure E (tan colored solid, 0.042 g, 32%). ¹H NMR (400 MHz, DMSO) δ 10.99 (s, 1H), 10.83 (s, 1H), 8.75 (d, J=1.6 Hz, 1H), 8.65 (d, J=6.1 Hz, 4H), 8.48 (dd, J=7.9, 1.6 Hz, 1H), 8.04-7.94 (m, 3H), 7.86-7.79 (m, 2H), 7.35-7.30 (m, 4H), 7.24 (dd, J=9.7, 6.8 Hz, 4H). ¹⁹F NMR (376 MHz, DMSO) δ −73.80. LCMS: 1.917 min, MS: ES+548.1.

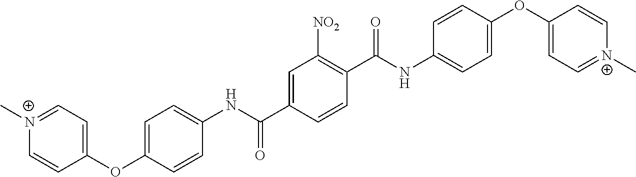

### Example 84

4,4′-((((2-nitroterephthaloyl)bis(azanediyl))bis(4,1-phenylene))bis(oxy))bis(1-methylpyridin-1-ium) (Example 84): Prepared from example 83 and methyl p-toluenesulfonate following the procedure D (light yellow solid, 0.047 g, 85%). ¹H NMR (400 MHz, DMSO) δ 11.27 (s, 1H), 11.20 (s, 1H), 8.85 (dd, J=7.0, 4.5 Hz, 4H), 8.79 (d, J=1.5 Hz, 1H), 8.58 (dd, J=7.9, 1.7 Hz, 1H), 8.10 (d, J=9.0 Hz, 2H), 7.99 (d, J=7.9 Hz, 1H), 7.93 (d, J=9.0 Hz, 2H), 7.57 (t, J=7.3 Hz, 4H), 7.42-7.32 (m, 4H), 4.23 (s, 6H). LCMS: 1.963 min, MS: ES+288.6.

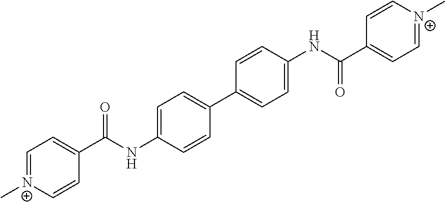

### Example 85

4,4′-(([1,1′-biphenyl]-4,4′-diylbis(azanediyl))bis(carbonyl))bis(1-methylpyridin-1-ium) (Example 85): Prepared from example 68 and methyl p-toluenesulfonate following the procedure D (orange solid, 0.061 g, 95%). ¹H NMR (400 MHz, DMSO) δ 9.08 (d, J=6.6 Hz, 4H), 8.48 (d, J=6.6 Hz, 4H), 7.86 (d, J=8.7 Hz, 4H), 7.76 (d, J=8.7 Hz, 4H), 4.39 (s, 6H). LCMS: 1.608 min, MS: ES+212.1.

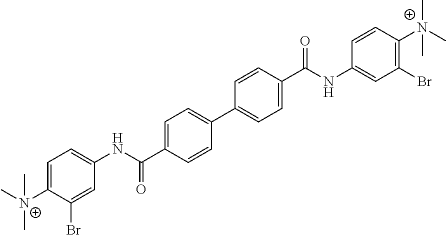

### Example 86

4,4′-(([1,1′-biphenyl]-4,4′-dicarbonyl)bis(azanediyl))bis(2-bromo-N,N,N-trimethylbenzenaminium) (Example 86): Prepared from example 80 and methyl p-toluenesulfonate following the procedure D (light grey solid, 0.055 g, 93%). ¹H NMR (400 MHz, DMSO) δ 8.43 (s, 2H), 8.09 (d, J=8.4 Hz, 4H), 8.01 (s, 4H), 7.94 (d, J=8.5 Hz, 4H), 3.79 (s, 18H). LCMS: 2.071 min, MS: ES+333.1.

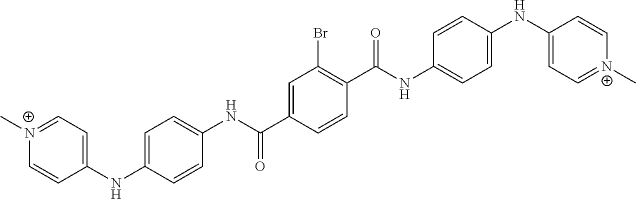

### Example 87

4,4′-((((2-bromoterephthaloyl)bis(azanediyl))bis(4,1-phenylene))bis(azanediyl))bis(1-methylpyridin-1-ium) (Example 87): Prepared from example 68 (SLUPP-2118) and methyl p-toluenesulfonate following procedure D (light yellow solid, 0.016 g, 30%). ¹H NMR (400 MHz, DMSO) δ 10.88 (s, 1H), 10.85 (s, 1H), 10.83 (s, 1H), 10.72 (s, 1H), 8.36 (d, J=1.4 Hz, 1H), 8.27 (dd, J=7.4, 2.1 Hz, 4H), 8.14 (dd, J=7.9, 1.5 Hz, 1H), 7.96 (d, J=8.9 Hz, 2H), 7.86 (d, J=8.8 Hz, 2H), 7.75 (d, J=7.9 Hz, 1H), 7.36 (dd, J=9.0, 2.5 Hz, 4H), 7.16 (d, J=5.6 Hz, 4H), 3.96 (s, 6H). LCMS: 1.970 min, MS: ES+304.1.

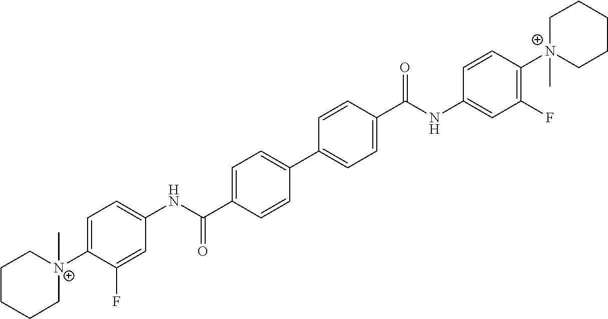 [SMILES: C[N+]1(c2ccc(NC(=O)c3ccc(-c4ccc(C(=O)Nc5ccc([N+]6(C)CCCCC6)c(F)c5)cc4)cc3)cc2F)CCCCC1]

### Example 88

1,1′-((([1,1′-biphenyl]-4,4′-dicarbonyl)bis(azanediyl))bis(2-bromo-4,1-phenylene))bis(1-methylpiperidin-1-ium) (Example 88): Prepared from N⁴,N^{4′}-bis(4-(piperidin-1-yl)phenyl)-[1,1′-biphenyl]-4,4′-dicarboxamide and methyl p-toluenesulfonate following the procedure D (tan color solid, 0.014 g, 27%). ¹H NMR (400 MHz, DMSO) δ 10.73 (s, 1H), 8.08 (d, J=8.4 Hz, 4H), 8.02 (dd, J=9.2, 1.7 Hz, 4H), 7.93 (d, J=8.4 Hz, 4H), 7.85 (d, J=9.4 Hz, 4H), 4.31 (d, J=10.7 Hz, 4H), 3.73 (t, J=10.0 Hz, 4H), 3.35 (s, 6H), 1.86 (t, J=14.6 Hz, 4H), 1.58 (s, 8H). LCMS: 2.125 min, MS: ES+294.2.

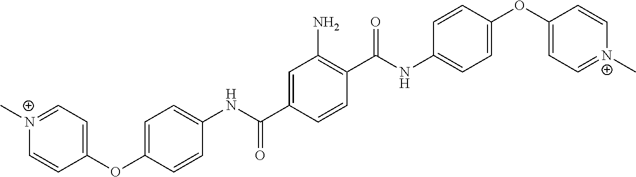

### Example 89

4,4′-((((2-aminoterephthaloyl)bis(azanediyl))bis(4,1-phenylene))bis(oxy))bis(1-methylpyridin-1-ium) (Example 89): Prepared from example 84 following procedure H (light yellow solid, 0.012 g, 29%). ¹H NMR (400 MHz, DMSO) δ 10.51 (s, 1H), 10.40 (s, 1H), 8.82 (d, J=7.4 Hz, 4H), 8.00 (d, J=9.1 Hz, 2H), 7.95 (d, J=9.1 Hz, 2H), 7.79 (d, J=8.3 Hz, 1H), 7.55 (d, J=7.0 Hz, 4H), 7.39-7.30 (m, 5H), 7.17 (dd, J=8.2, 1.6 Hz, 1H), 6.57 (s, 2H), 4.21 (s, 6H). ¹⁹F NMR (376 MHz, DMSO) δ −73.46. LCMS: 1.862 min, MS: ES+273.6.

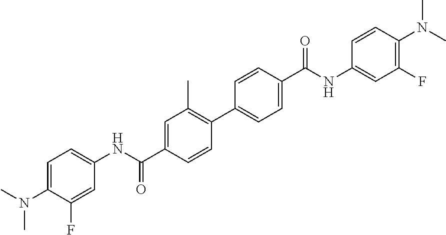

### Example 90

N⁴,N^{4′}-bis(4-(dimethylamino)-3-fluorophenyl)-2-methyl-[1,1′-biphenyl]-4,4′-dicarboxamide (Example 90): Prepared from 2-methyl-[1,1′-biphenyl]-4,4′-dicarboxylic acid and 2-fluoro-N¹,N¹-dimethylbenzene-1,4-diamine following the procedure C (tan colored solid, 0.123 g, 96%.). ¹H NMR (400 MHz, DMSO) δ 10.36 (s, 1H), 10.31 (s, 1H), 8.04 (d, J=8.3 Hz, 2H), 7.92 (d, J=1.1 Hz, 1H), 7.87 (dd, J=8.0, 1.4 Hz, 1H), 7.73 (dd, J=15.2, 1.1 Hz, 2H), 7.56 (d, J=8.3 Hz, 2H), 7.50 (d, J=8.8 Hz, 2H), 7.41 (d, J=7.9 Hz, 1H), 7.07 (t, J=9.5 Hz, 2H), 2.80 (s, 12H), 2.35 (s, 3H). ¹⁹F NMR (376 MHz, DMSO) δ −74.89, −121.28. LCMS: 2.193 min, MS: ES+529.2.

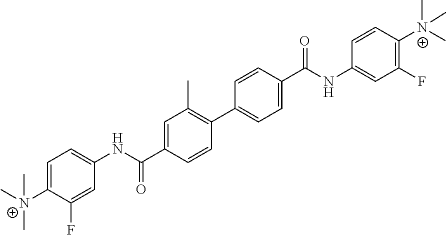

### Example 91

4,4′-((2-methyl-[1,1′-biphenyl]-4,4′-dicarbonyl)bis(azanediyl))bis(2-fluoro-N,N,N-trimethylbenzenaminium) (Example 91): Prepared from example 90 and methyl p-toluenesulfonate following the procedure D (white solid, 0.033 g, 67%). ¹H NMR (400 MHz, DMSO) δ 10.90 (s, 1H), 10.85 (s, 1H), 8.14-8.05 (m, 4H), 7.96 (s, 1H), 7.91 (dd, J=12.1, 7.0 Hz, 3H), 7.79 (d, J=9.2 Hz, 2H), 7.58 (d, J=8.3 Hz, 2H), 7.43 (d, J=8.0 Hz, 1H), 3.67 (s, 18H), 2.35 (s, 3H). LCMS: 2.110 min, MS: ES+279.2.

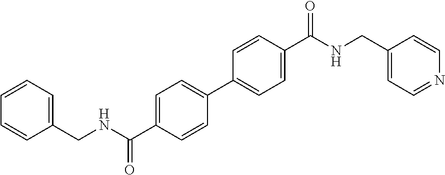 [SMILES: O=C(NCc1ccccc1)c1ccc(-c2ccc(C(=O)NCc3ccncc3)cc2)cc1]

### Example 92

N⁴,N^{4′}-bis(pyridin-4-ylmethyl)-[1,1′-biphenyl]-4,4′-dicarboxamide (Example 92): Prepared from [1,1′-biphenyl]-4,4′-dicarboxylic acid and pyridin-4-ylmethanamine following procedure C (white solid, 0.057 g, 53%.). ¹H NMR (400 MHz, DMSO) δ 9.36 (t, J=5.8 Hz, 2H), 8.73 (d, J=6.4 Hz, 4H), 8.05 (d, J=8.4 Hz, 4H), 7.91 (d, J=8.4 Hz, 4H), 7.73 (d, J=6.2 Hz, 4H), 4.68 (d, J=5.8 Hz, 4H). ¹⁹F NMR (376 MHz, DMSO) δ −74.17. LCMS: 1.602 min, MS: ES+423.1.

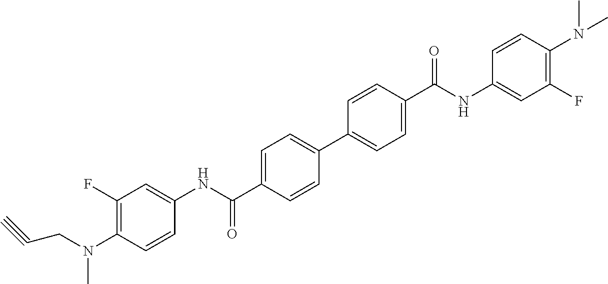 [SMILES: C#CCN(C)c1ccc(NC(=O)c2ccc(-c3ccc(C(=O)Nc4ccc(N(C)C)c(F)c4)cc3)cc2)cc1F]

### Example 93

### N⁴-(4-(dimethylamino)-3-fluorophenyl)-N^{4′}-(3-fluoro-4-(methyl(prop-2-yn-1-yl)amino)phenyl)-[1,1′-biphenyl]-4,4′-dicarboxamide (Example 93)

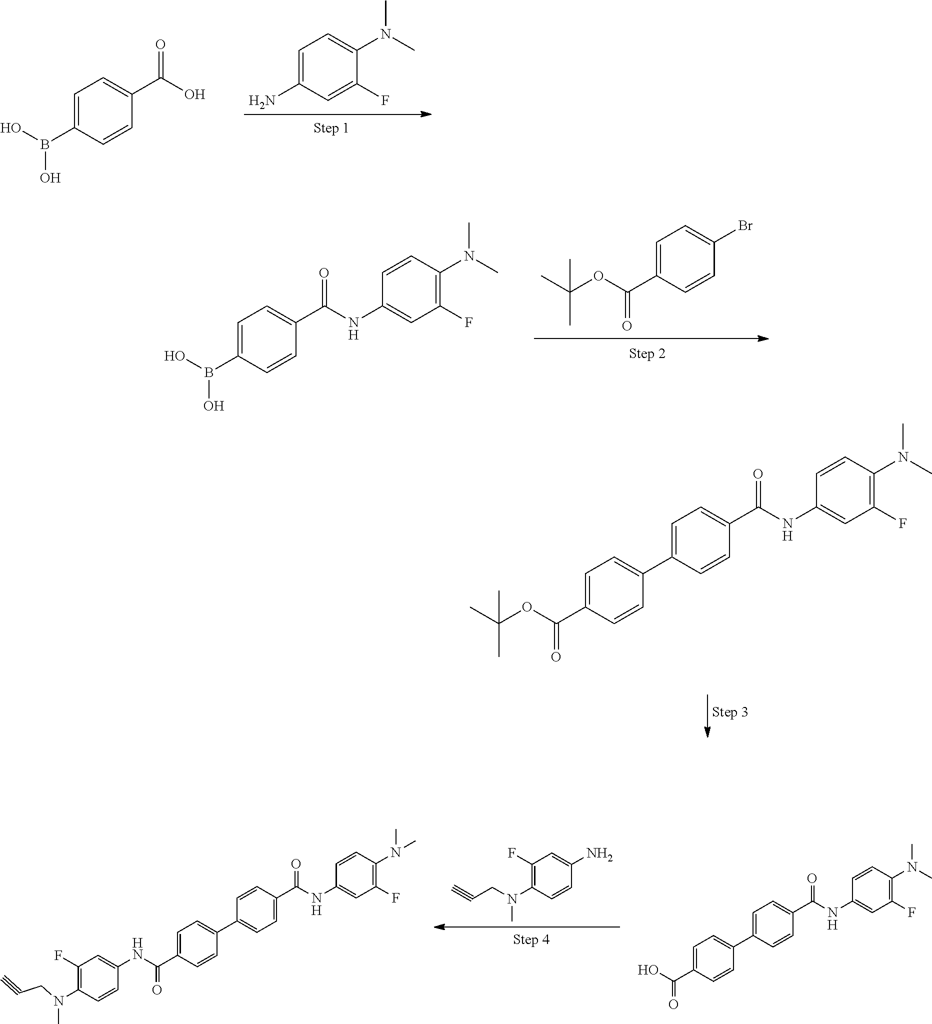

Step 1: To a mixture of 4-Boronic benzoic acid (1.00 g, 6.03 mmol) and 2-Fluoro-N-1-,N-1-dimethyl-1,4-benzenediamine (1.02 g, 6.63 mmol) in DMF (15 mL) at room temperature was added diisopropylethylamine (2.15 mL, 12.06 mmol) followed by TBTU (2.32 g, 7.24 mmol). The resulting mixture was stirred at room temperature overnight, at which time water was added to the reaction mixture. The precipitate that developed was collected by filtration. The solids were washed with water and then hexanes to give the product boronic acid (1.70 g, 93%) as a grey solid. ¹H-NMR (400 MHz, DMSO-d₆) δ ppm 10.26 (s, 1H), 8.29 (s, 2H), 7.96-7.91 (m, 4H), 7.73-7.69 (dd, J=2.3, 15.4 Hz, 1H), 7.51-7.48 (dd, J=1.8, 8.6 Hz, 1H), 7.03-6.98 (dd, J=9.0, 10.0 Hz, 1H), 2.78 (s, 6H). LCMS: 1.54 min, MS: ES+303.1.

Step 2: The above boronic acid (0.500 g, 1.66 mmol), tert-buty-4-bromobenzene (0.387 g, 1.50 mmol) and K₂CO₃ (0.688 g, 4.98 mmol) were combined in toluene (16 mL) and methanol (4.0 mL) and the mixture was purged with argon. To the mixture was added Pd(PPh₃)₄ (96 mg, 0.083 mmol). The resulting mixture was heated to 80° C. for 2 hours. After cooling to room temperature, the mixture was concentrated to dryness, then the residue was dissolved in hot chloroform and filtered through a pad of Celite®. The filtrate was concentrated and then treated with methanol, the solids were collected by filtration to give the bi-phenyl product (0.476 g, 73%) as a pale-yellow solid. A portion of crude product (200 mg) was dissolved in CHCl₃, absorbed onto silica gel and subjected to silica gel chromatography (dichloromethane:methanol, 5-10%) to give the product (0.190 g, 95%). ¹H-NMR (400 MHz, DMSO-d₆) δ ppm 10.30 (s, 1H), 8.08 (d, J=8.4 Hz, 2H), 8.02 (d, J=8.4 Hz, 2H), 7.92-7.89 (m, 4H), 7.75-7.71 (dd, J=2.3, 15.4 Hz, 1H), 7.49-7.46 (m, 1H), 7.01-6.96 (m, 1H), 2.76 (s, 6H), 1.58 (s, 9H). LCMS: 2.78 min, MS: ES+435.2

Step 3: The ester (0.300 g, 0.690 mmol) was taken up in dichloromethane (3.0 mL) and treated with trifluoroacetic acid (1.0 mL). After stirring at room temperature for 3.5 hours the reaction was concentrated to dryness. The resulting solids were washed with dichloromethane to give the desired acid (0.391 g, >100%) as an off-white solid. ¹H-NMR (400 MHz, DMSO-d₆) δ ppm 10.39 (s, 1H), 8.12-8.09 (m, 4H), 7.96-7.91 (m, 4H), 7.75-7.71 (dd, J=1.4, 15.4 Hz, 1H), 7.54 (d, J=8.8 Hz, 1H), 7.14-7.09 (m, 1H), 2.84 (s, 6H). LCMS: 2.07 min, MS: ES+379.1.

Step 4: To a suspension of the above acid (0.147 g, 0.299 mmol) in DMF (1.0 mL) was added diisopropylethylamine (0.267 mL, 1.49 mmol) followed by PyOxim (0.166 g, 0.314 mmol) and the resulting mixture was stirred at room temperature for 30 minutes at which time 2-fluoro-N¹-methyl-N¹-(prop-2-yn-1-yl)benzene-1,4-diamine (0.059 g, 0.329 mmol), made in 2 steps from 3,4-difluoro-nitrobenzene and N-methylpropargylamine, was added. After stirring at room temperature for 3.0 hours additional amine (0.027 g, 0.150 mmol). After 1 hour at room temperature additional PyOxim (0.079 g, 0.150 mmol) was added to the mixture which was stirred at room temperature overnight. Additional PyOxim (0.032 g, 0.060 mmol) was added to the mixture which was stirred at room temperature for 1 hour. Water was added to the reaction mixture and the precipitate that formed was collected by filtration, washing with water and then diethyl ether to give the coupled product (0.158 g, 98%) as a flesh colored solid. ¹H-NMR (400 MHz, DMSO-d₆) δ ppm 10.38 (s, 1H), 10.34 (s, 1H), 8.14-8.11 (m, 4H), 7.99-7.97 (m, 4H), 7.79-7.71 (m, 2H), 7.56-7.51 (m, 2H), 7.14-7.10 (m, 1H), 7.05-7.00 (m, 1H), 3.98 (br s, 2H), 3.20 (m, 1H), 2.82 (s, 3H), 2.79 (s, 6H). LCMS: 2.58 min, MS: ES+539.2.

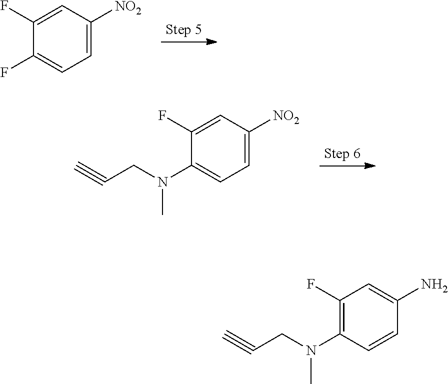

Step 5: 3,4-Difluoro-nitrobenzene (0.500 g, 3.14 mmol) and N-methylpropargylamine (0.435 g, 6.29 mmol) were combined and stirred at room temperature overnight. Additional N-methylpropargylamine (0.059 g, 0.854 mmol) was added, and the resulting mixture was stirred at room temperature for 24 hours. The reaction was diluted with water and extracted into DCM. The combined organics were dried (Na₂SO₄) filtered and concentrated to give the product (0.618 g, 94%) as a yellow foam. ¹H-NMR (400 MHz, DMSO-d₆) δ ppm 8.08-8.07 (m, 1H), 8.05-8.04 (m, 1H), 7.16 (t, J=9.0 Hz, 1H), 4.25 (d, J=2.4 Hz, 2H), 3.34 (t, J=2.4 Hz, 1H), 3.07 (d, J=1.7 Hz, 3H). LCMS: 2.56 min, MS: ES+209.0.

Step 6: To a mixture of nitrobenzene (0.600 g, 2.88 mmol) in THF (1 mL), ethanol (1 mL) and water (1 mL) was added iron (0.804 g, 14.40 mmol) and acetic acid (0.824 mL, 14.40 mmol). The mixture was heated to 50° C. for 2 hours. After cooling to room temperature, the reaction mixture was filtered through a pad of Celite® washing with ethanol and then saturated NaHCO₃ solution. The filtrate was partially concentrated, and water was added. The aqueous reaction mixture was extracted into ethyl acetate. The combined organics were washed with brine, dried (Na₂SO₄), filtered and concentrated. The resulting oil was purified by silica gel chromatography (hexanes-ethyl acetate, 1:0 to 7:3) to give the product (0.417 g, 81%) as a brown oil. ¹H-NMR (400 MHz, DMSO-d₆) δ ppm 6.87-6.83 (m, 1H), 6.38-6.31 (m, 2H), 5.04 (br s, 2H), 3.75 (d, J=2.3 Hz, 2H), 3.14 (t, J=2.3 Hz, 1H), 2.66 (s, 3H). LCMS: 1.15 min, MS: ES+179.1.

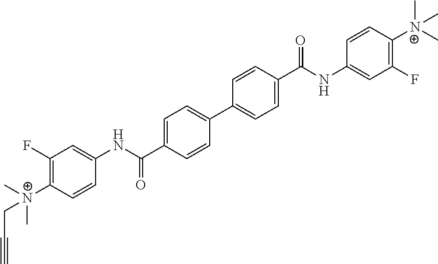 [SMILES: C#CC[N+](C)(C)c1ccc(NC(=O)c2ccc(-c3ccc(C(=O)Nc4ccc([N+](C)(C)C)c(F)c4)cc3)cc2)cc1F]

### Example 94

4-(4′-((4-(dimethyl(prop-2-yn-1-yl)ammonio)-3-fluorophenyl)carbamoyl)-[1,1′-biphenyl]-4-carboxamido)-2-fluoro-N,N,N-trimethylbenzenaminium (Example 94): To example 93 (0.059 g, 0.110 mmol) and Methyl p-toluenesulfonate (0.204 g, 1.10 mmol) were combined in DMF (0.5 mL) and heated to 80° C. overnight. Additional Methyl p-toluenesulfonate (0.102 g, 0.550 mmol) was added and the resulting mixture was stirred at 80° C. for 5 days. Additional Methyl p-toluenesulfonate (0.102 g, 0.550 mmol) was added and the resulting mixture was stirred at 80° C. for 3 days at which time the reaction mixture was concentrated by blowing air over the mixture at room temperature. Water was added to the residue and the precipitate was collected by filtration, washing with water and then diethyl ether. The product was dried under vacuum overnight to give the desired product (0.084 g, 84%) as a flesh colored solid. ¹H-NMR (400 MHz, DMSO-d₆) δ ppm 10.89 (s, 1H), 10.86 (s, 1H), 8.19-8.13 (m, 6H), 8.04-7.97 (m, 6H), 7.86-7.82 (m, 2H), 7.52-7.51 (d, J=8.0 Hz, 4H), 7.16-7.1 (d, J=7.8 Hz, 4H), 5.01 (m, 2H), 4.02 (m, 1H), 3.77 (s, 6H), 3.73 (s, 9H), 2.32 (s, 6H). LCMS: 2.11 min, MS: ES+284.2

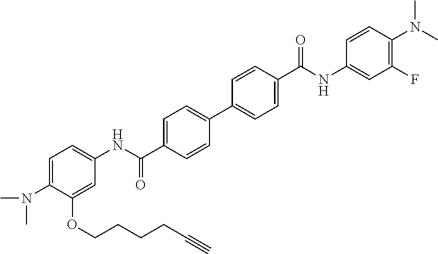

### Example 95

### N⁴-(4-(dimethylamino)-3-(hex-5-yn-1-yloxy)phenyl)-N^{4′}-(4-(dimethylamino)-3-fluorophenyl)-[1,1′-biphenyl]-4,4′-dicarboxamide (Example 95)

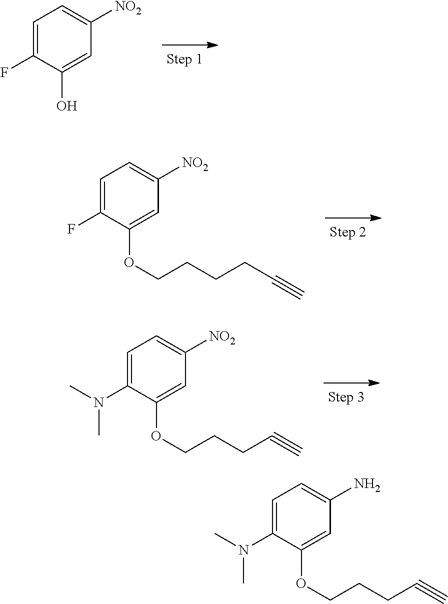

Step 1: 4-Fluoro-3-hydroxy-nitrobenzene (0.650 g, 4.14 mmol), Cs₂CO₃ (2.70 g, 8.28 mmol) and 1-bromo-5-hexyne (0.800 g, 4.97 mmol) were combined in DMF (10 mL) and stirred at room temperature overnight. The reaction was added to water and extracted into ethyl acetate. The combined organics were washed with brine, dried (Na₂SO₄) filtered and concentrated to give the product (0.972 g, 99%) as an amber oil. ¹H-NMR (400 MHz, DMSO-d₆) δ ppm 8.02-7.99 (m, 1H), 7.94-7.90 (m, 1H), 7.57-7.53 (dd, J=9.0, 10.5 Hz, 1H), 4.26 (t, J=6.4 Hz, 2H), 2.81 (t, J=2.6 Hz, 1H), 2.32-2.27 (dt, J=2.6, 7.1 Hz, 2H), 1.93-1.86 (m, 2H), 1.69-1.62 (m, 2H). LCMS: 2.81 min, MS: ES+ No ionization.

Step 2: To a solution of nitrobenzene compound (0.950 g, 4.00 mmol) in ethanol (5.0 mL), in a microwave vial was added a solution of N,N-dimethylamine in THF (6.00 mL, 2M in THF, 12.01 mmol). The vial was sealed and stirred at room temperature overnight. The reaction was partially concentrated then additional N,N-dimethylamine in THF (6.00 mL, 2M in THF, 12.01 mmol) was added and the vial was sealed again and stirred at room temperature overnight. The crude reaction mixture was concentrated and subjected to silica gel chromatography (hexanes-ethyl acetate, 1:0 to 4:1) to provide the product (0.851 g, 82%) as an orange oil. ¹H-NMR (400 MHz, DMSO-d₆) δ ppm 7.85-7.83 (dd, J=2.4, 9.0 Hz, 1H), 7.68 (d, J=2.5 Hz, 1H), 6.90 (d, J=9.0 Hz, 1H), 4.13 (t, J=6.3 Hz, 2H), 3.01 (s, 6H), 2.83 (t, J=2.6 Hz, 1H), 2.32-2.27 (td, J=2.6, 7.1 Hz, 2H), 1.95-1.88 (m, 2H), 1.72-1.65 (m, 2H). LCMS: 2.66 min, MS: ES+263.1.

Step 3: To a mixture of nitro aniline compound (0.262 g, 1.00 mmol) in THF (1 mL), ethanol (1 mL) and water (1 mL) was added iron (0.279 g, 5.00 mmol) and acetic acid (0.286 mL, 5.00 mmol). The mixture was heated to 50° C. for 1 hour. After cooling to room temperature, the reaction mixture was filtered through a pad of Celite® washing with methanol and then saturated NaHCO₃ solution. The filtrate was partially concentrated, and water was added. The aqueous reaction mixture was extracted into ethyl acetate. The combined organics were washed with brine, dried (Na₂SO₄), filtered and concentrated. The resulting oil was purified by silica gel chromatography (hexanes-ethyl acetate, 8:2 to 0:1) to give the product (0.106 g, 46%) as a dark red/purple oil. ¹H-NMR (400 MHz, DMSO-d₆) δ ppm 6.63 (d, J=8.3 Hz, 1H), 6.23 (d, J=2.3 Hz, 1H), 6.11-6.08 (dd, J=2.4, 8.3 Hz, 1H), 4.66 (br s, 2H), 3.90 (t, J=6.2 Hz, 2H), 2.82 (t, J=2.6 Hz, 1H), 2.58 (s, 6H), 2.30-2.26 (td, J=2.6, 7.1 Hz, 2H), 1.87-1.81 (m, 2H), 1.70-1.63 (m, 2H). LCMS: 1.56 min, MS: ES+233.2.

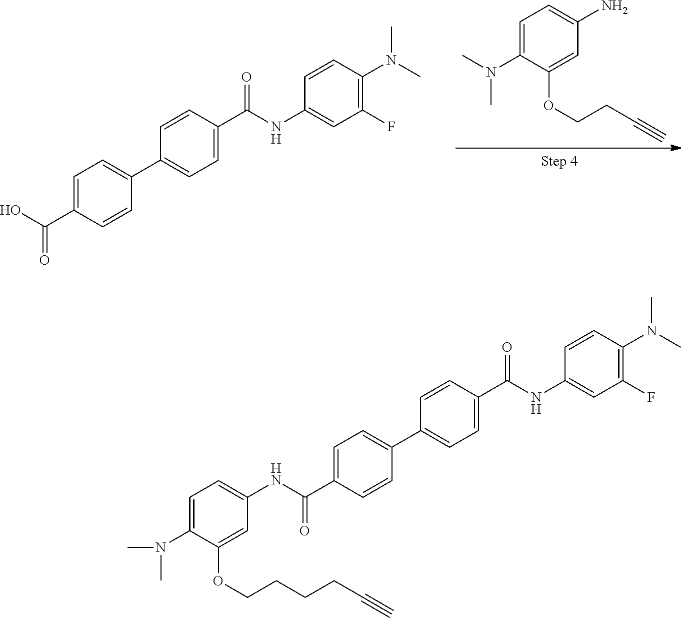

Step 4: Example 95 was prepared from the amine isolated in step 3 above and the acid obtained in step 3 in the preparation of example 93 according to the step 4 of the procedure outlined for example 93. The product (0.125 g, 61%) was isolated as a yellow/green solid. ¹H-NMR (400 MHz, DMSO-d₆) δ ppm 10.34 (s, 1H), 10.17 (s, 1H), 8.13-8.12 (m, 4H), 7.98-7.96 (m, 4H), 7.75-7.72 (d, J=15.0 Hz, 1H), 7.52-7.50 (m, 2H), 7.38 (d, J=8.5 Hz, 1H), 7.02 (t, J=9.4 Hz, 1H), 6.89 (d, J=8.5 Hz, 1H), 4.04-4.01 (m, 2H), 2.85-2.76 (m, 7H), 2.76-2.68 (m, 6H), 2.35-2.26 (m, 2H), 1.97-1.86 (m, 2H), 1.77-1.66 (m, 2H). LCMS: 2.34 min, MS: ES+593.2.

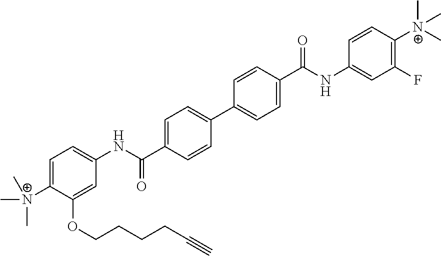

### Example 96

2-fluoro-4-(4′-((3-(hex-5-yn-1-yloxy)-4-(trimethylammonio)phenyl)carbamoyl)-[1,1′-biphenyl]-4-carboxamido)-N,N,N-trimethylbenzenaminium (Example 96): The desired product was prepared from example 95 according to the procedure outlined for example 94. The product (0.087 g, 86%) was isolated as a pale pink solid. ¹H-NMR (400 MHz, DMSO-d₆) δ ppm 10.86 (s, 1H), 10.65 (s, 1H), 8.25-8.10 (m, 5H), 8.07-7.95 (m, 6H), 7.87-7.78 (m, 2H), 7.69-7.63 (d, J=7.8 Hz, 2H), 7.51 (d, J=7.8 Hz, 1H), 7.14 (d, J=7.6 Hz, 1H), 4.32-4.24 (m, 2H), 3.72 (s, 9H), 3.70 (s, 9H), 2.90-2.84 (m, 1H), 2.39-2.26 (m, 8H), 2.10-1.99 (m, 2H), 1.77-1.66 (m, 2H). LCMS: 2.36 min, MS: ES+311.2.

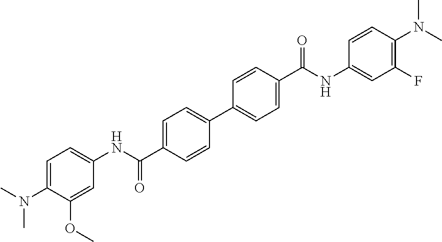

### Example 97

N⁴-(4-(dimethylamino)-3-fluorophenyl)-N^{4′}-(4-(dimethylamino)-3-methoxyphenyl)-[1,1′-biphenyl]-4,4′-dicarboxamide (Example 97): was prepared from the acid obtained in step 3 of example 93 and commercially available 2-methoxy-N¹,N¹-dimethylbenzene-1,4-diamine dihydrochloride according to the step 4 of the procedure outlined for example 94. The product (0.045 g, 70%) was isolated as a yellow/green solid. ¹H-NMR (400 MHz, DMSO-d₆) δ ppm 10.34 (s, 1H), 10.20 (s, 1H), 8.14-8.11 (m, 4H), 7.99-7.96 (m, 4H), 7.76-7.71 (m, 1H), 7.52 (br m, 2H), 7.40-7.37 (m, 1H), 7.05-7.00 (m, 1H), 6.89 (d, J=8.6 Hz, 1H), 3.84 (s, 3H), 2.79 (s, 6H), 2.71 (s, 6H). LCMS: 2.06 min, MS: ES+527.2.

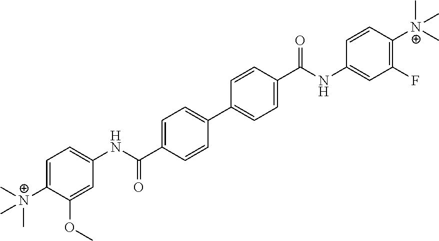

### Example 98

2-fluoro-4-(4′-((3-methoxy-4-(trimethylammonio)phenyl)carbamoyl)-[1,1′-biphenyl]-4-carboxamido)-N,N,N-trimethylbenzenaminium (Example 98): was prepared from example 97 according to the procedure outlined for example 93. The product (0.032 g, 63%) was isolated as a pink solid. ¹H-NMR (400 MHz, DMSO-d₆) δ ppm 10.86 (s, 1H), 10.67 (s, 1H), 8.19-8.12 (m, 5H), 8.04-7.95 (m, 6H), 7.84-7.81 (m, 2H), 7.68-7.65 (m, 1H), 7.51 (d, J=8.0 Hz, 1H), 7.14 (d, J=7.8 Hz, 1H), 4.07 (s, 3H), 3.72 (s, 9H), 3.70 (s, 9H). LCMS: 2.09 min, MS: ES+278.2.

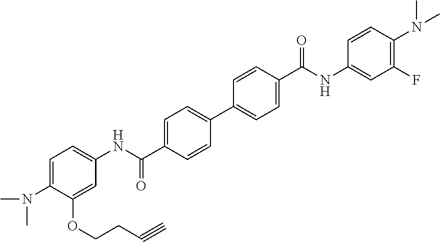

### Example 99

### N⁴-(3-(but-3-yn-1-yloxy)-4-(dimethylamino)phenyl)-N^{4′}-(4-(dimethylamino)-3-fluorophenyl)-[1,1′-biphenyl]-4,4′-dicarboxamide (Example 99)

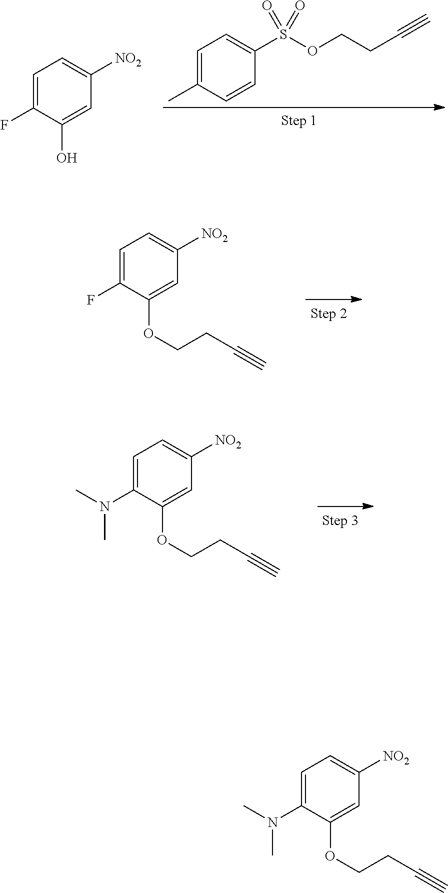 [SMILES: O=[N+]([O-])c1ccc(F)c(O)c1 ; C#CCCOS(=O)(=O)c1ccc(C)cc1 ; C#CCCOc1cc([N+](=O)[O-])ccc1F ; C#CCCOc1cc([N+](=O)[O-])ccc1N(C)C ; C#CCCOc1cc([N+](=O)[O-])ccc1N(C)C]

Step 1: 4-Fluoro-3-hydroxy-nitrobenzene (0.5000 g, 3.18 mmol), Cs₂CO₃ (2.07 g, 6.36 mmol), KI (0.053 g. 0.318 mmol) and but-3-yn-1-yl 4-methylbenzenesulfonate, prepared according to the procedure described in U.S. Patent Application No. 2016/0347717 (0.856 g, 3.82 mmol) were combined in DMF (7.0 mL) and stirred at 80° C. for 1.5 h. The reaction was added to water and the resulting mixture was stirred at room temperature overnight. The precipitate that formed was collected by filtration to give the crude product (0.435 g, 65%) as a yellow solid which was used without further purification. LCMS: 2.48 min. MS ES+ No ionization.

Step 2: To a solution of nitrobenzene (0.430 g, 2.06 mmol) in ethanol (2.5 mL), in a microwave vial was added a solution of N,N-dimethylamine in THF (3.08 mL, 2M in THF, 6.17 mmol). The vial was sealed and stirred at room temperature overnight. After stirring at room temperature overnight, by TLC the reaction was complete. The crude reaction mixture was concentrated. DCM was added to the resulting residue, which was filtered to remove the insoluble material. The filtrate was concentrated to provide the crude product (0.341 g, 70%) as a yellow/orange solid, which was used without further purification. LCMS: 2.31 min, MS: ES+235.1.

Step 3: To a mixture of nitroaniline (0.341 g, 146 mmol) in THF (1 mL), ethanol (1 mL) and water (1 mL) was added iron (0.407 g, 7.28 mmol) and acetic acid (0.416 mL, 7.28 mmol). The mixture was heated to 50° C. for 1 hour. After cooling to room temperature, the reaction mixture was filtered through a pad of Celite® washing with DCM, then methanol and then saturated NaHCO₃ solution. The filtrate was partially concentrated, and water was added. Extraction of the aqueous reaction mixture was attempted with ethyl acetate however the dark layers did not separate. The mixture was concentrated to remove the organics and chloroform was added. The mixture was filtered through a pad of Celite® washing with chloroform and the aqueous layer failed to elute through the pad of Celite®. The filtrate was concentrated and the resulting residue was purified by silica gel chromatography (dichloromethane:methanol, 0 to 10%) to give the product TB-A0037-041-1 (0.160 g, 63%) as a dark red/purple oil. ¹H-NMR (400 MHz, DMSO-d₆) δ ppm 6.64 (d, J=8.3 Hz, 1H), 6.25 (d, J=2.3 Hz, 1H), 6.15-6.11 (dd, J=2.4, 8.3 Hz, 1H), 4.70 (br s, 2H), 3.99 (t, J=6.5 Hz, 2H), 2.91 (t, J=2.6 Hz, 1H), 2.68-2.64 (td, J=2.6, 6.5 Hz, 2H), 2.59 (s, 6H). LCMS: 0.71 min, MS: ES+205.1.

Step 4: example 99 was prepared from the acid obtained in step 3 of example 93 and the above 1,4-diamine according to the procedure outlined for example 94. The product (0.115 g, 50%) was isolated as a mustard colored solid. ¹H-NMR (400 MHz, DMSO-d₆) δ ppm 10.33 (s, 1H), 10.18 (s, 1H), 8.14-8.11 (m, 4H), 7.99-7.97 (m, 4H), 7.76-7.71 (m, 1H), 7.53-7.51 (m, 2H), 7.41 (d, J=8.4 Hz, 1H), 7.05-7.00 (m, 1H), 6.90 (d, J=8.6 Hz, 1H), 4.10 (t, J=6.5 Hz, 2H), 2.95-2.93 (m, 1H), 2.79-2.75 (m, 14H). LCMS: 2.22 min, MS: ES+565.2.

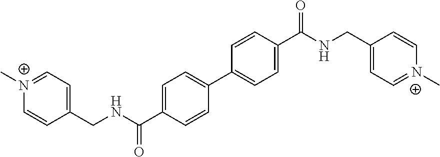

### Example 100

4,4′-((([1,1′-biphenyl]-4,4′-dicarbonyl)bis(azanediyl))bis(methylene))bis(1-methylpyridin-1-ium) (Example 100): Prepared from example 92 and methyl p-toluenesulfonate following procedure D (light yellow solid, 0.035 g, 70%). ¹H NMR (400 MHz, DMSO) δ 9.67 (t, J=5.7 Hz, 2H), 8.90 (d, J=6.6 Hz, 4H), 8.10 (d, J=8.4 Hz, 4H), 8.04 (d, J=6.6 Hz, 4H), 7.91 (d, J=8.4 Hz, 4H), 4.75 (d, J=5.5 Hz, 4H), 4.31 (s, 6H). LCMS: 1.624 min, MS: ES+226.1.

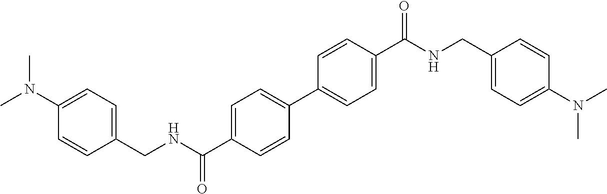

### Example 101

N⁴,N^{4′}-bis(4-(dimethylamino)benzyl)-[1,1′-biphenyl]-4,4′-dicarboxamide (Example 101): Prepared from [1,1′-biphenyl]-4,4′-dicarboxylic acid and 4-(aminomethyl)-N,N-dimethylaniline following procedure J (white solid, 0.035 g, 35%). ¹H NMR (400 MHz, DMSO) δ 8.97 (t, J=5.6 Hz, 2H), 7.99 (d, J=8.1 Hz, 4H), 7.83 (d, J=8.1 Hz, 4H), 7.16 (d, J=8.4 Hz, 4H), 6.69 (d, J=8.4 Hz, 4H), 4.38 (d, J=5.6 Hz, 4H), 2.85 (s, 12H). LCMS: 1.837 min, MS: ES+254.2.

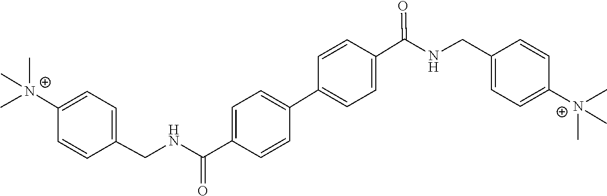

### Example 102

4,4′-((([1,1′-biphenyl]-4,4′-dicarbonyl)bis(azanediyl))bis(methylene))bis(N,N,N-trimethylbenzenaminium) (Example 102): Prepared from example 102 and methyl p-toluenesulfonate following the procedure D (white solid, 0.047 g, 78%). ¹H NMR (400 MHz, DMSO) δ 9.30 (t, J=6.0 Hz, 2H), 7.99 (d, J=8.4 Hz, 4H), 7.87 (dd, J=13.5, 8.7 Hz, 8H), 7.55 (d, J=8.9 Hz, 4H), 4.54 (d, J=4.9 Hz, 4H), 3.56 (s, 18H). LCMS: 1.818 min, MS: ES+268.2.

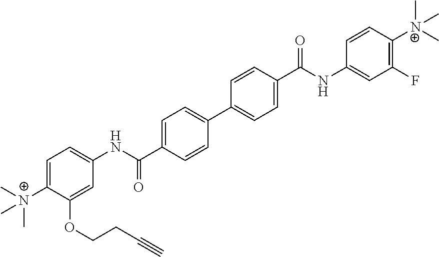

### Example 103

2-(but-3-yn-1-yloxy)-4-(4′-((3-fluoro-4-(trimethylammonio)phenyl)carbamoyl)-[1,1′-biphenyl]-4-carboxamido)-N,N,N-trimethylbenzenaminium (Example 103): Prepared from example 99 according to the procedure outlined for example 93. The product (0.135 g, 78%) was isolated as a flesh colored solid. ¹H-NMR (400 MHz, DMSO-d₆) δ ppm 10.86 (s, 1H), 10.66 (s, 1H), 8.19-8.13 (m, 5H), 8.04-7.97 (m, 5H), 7.92 (m, 1H), 7.86-7.82 (m, 2H), 7.69-7.66 (m, 1H), 7.51 (d, J=8.0 Hz, 4H), 7.14 (d, J=7.9 Hz, 4H), 4.34 (t, J=6.0 Hz, 2H), 3.74 (s, 9H), 3.73 (s, 9H), 3.06 (t, J=2.5 Hz, 1H), 2.95-2.92 (m, 2H), 2.32 (s, 6H). LCMS: 2.21 min, MS: ES+297.2, 579.2.

### Additional Compounds

TABLE 1
Additional Compounds

| Example | |
|---|---|
| # | Structure |
| 104 | 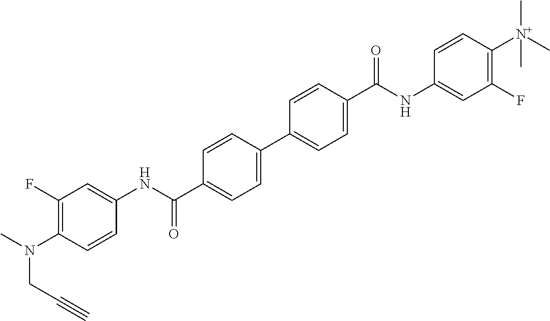 |
| 105 | 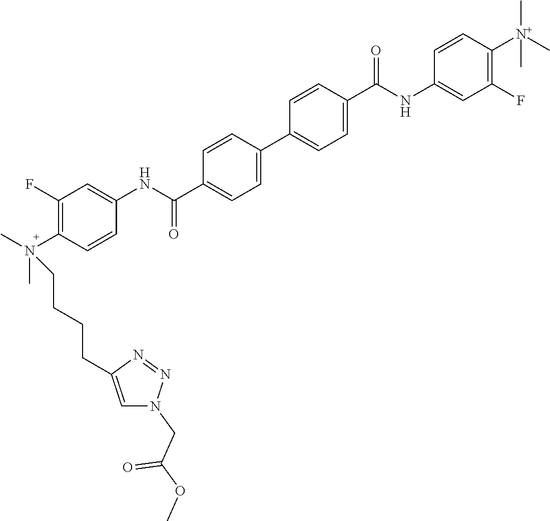 |
| 106 | 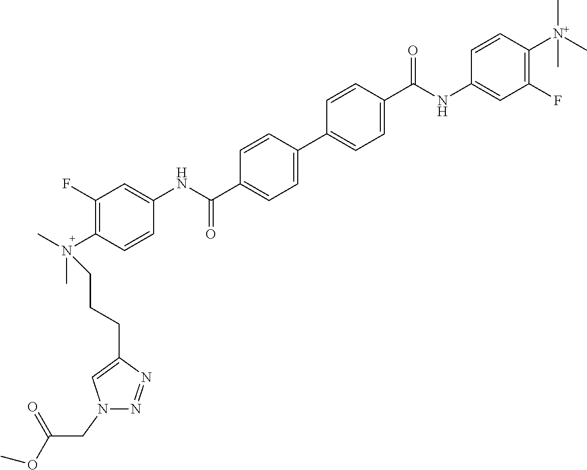 |
| 107 | 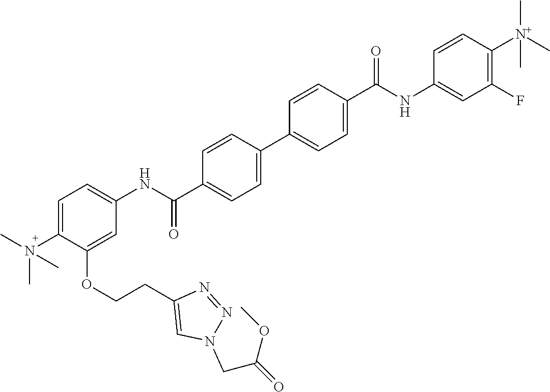 |
| 108 | 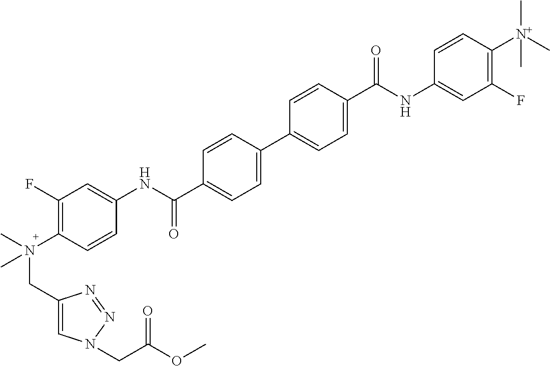 |
| 109 | 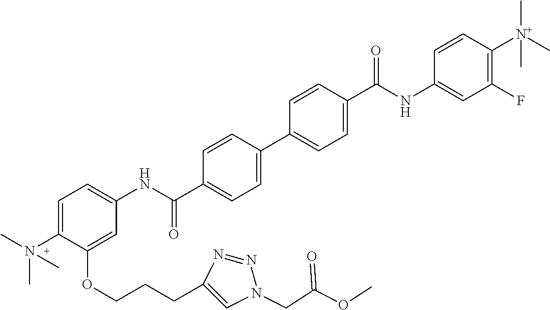 |
| 110 | 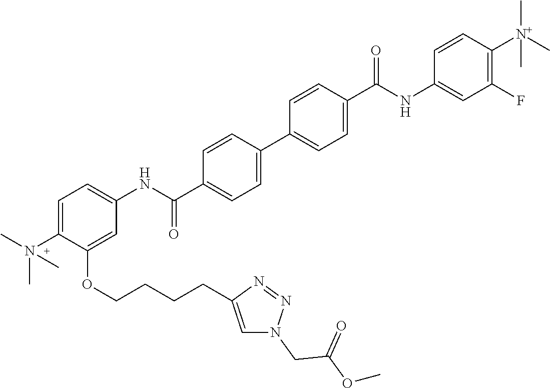 |
| 111 | 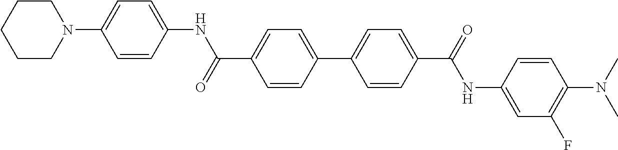 [SMILES: CN(C)c1ccc(NC(=O)c2ccc(-c3ccc(C(=O)Nc4ccc(N5CCCCC5)cc4)cc3)cc2)cc1F] |
| 112 | 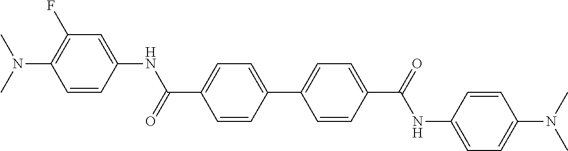 |
| 113 | 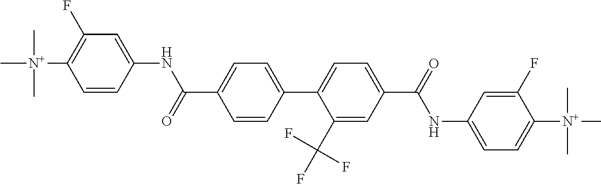 |
| 114 | 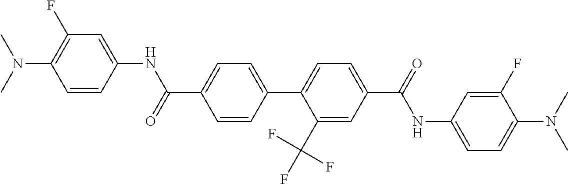 |
| 115 | 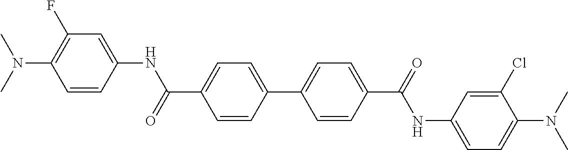 |
| 116 | 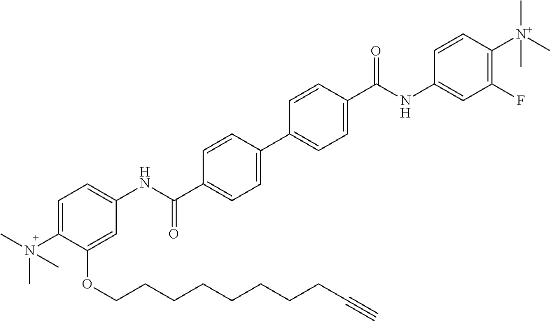 |
| 117 | 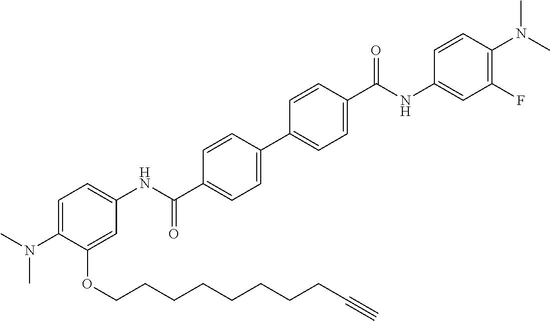 |
| 118 | 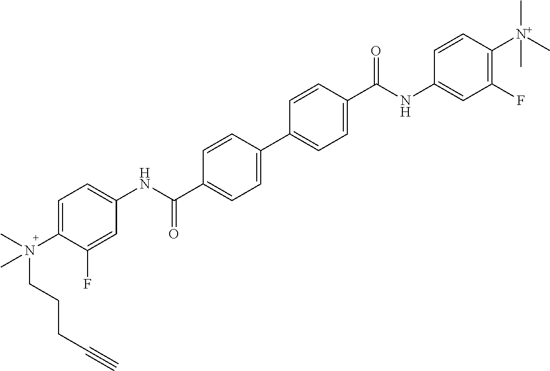 |
| 119 | 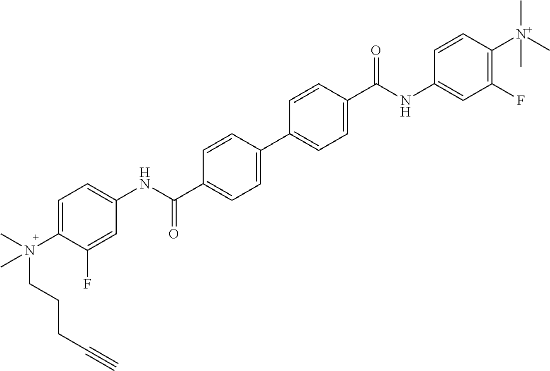 |
| 120 | 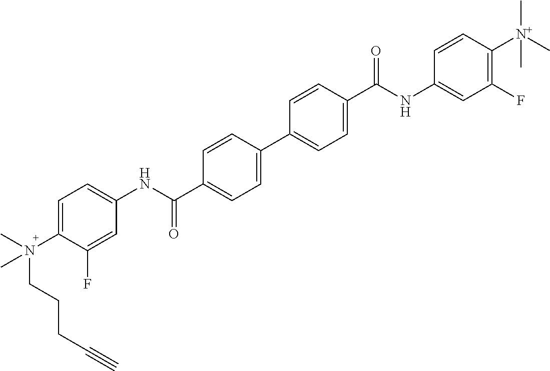 |
| 121 | 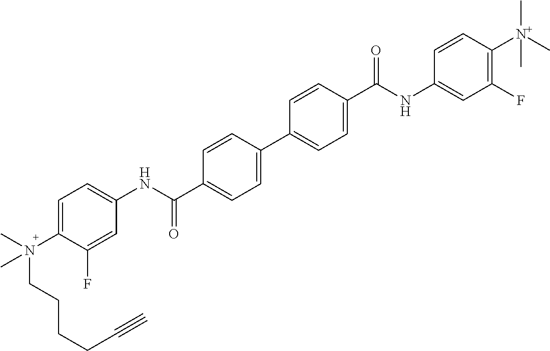 |
| 122 | 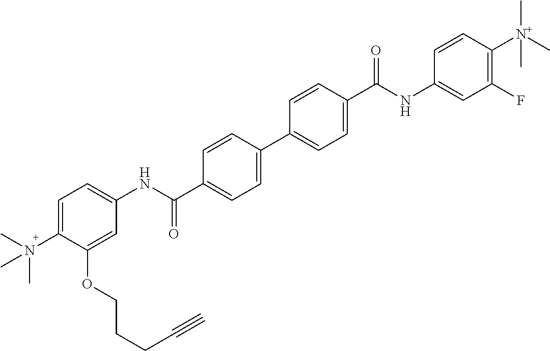 |
| 123 | 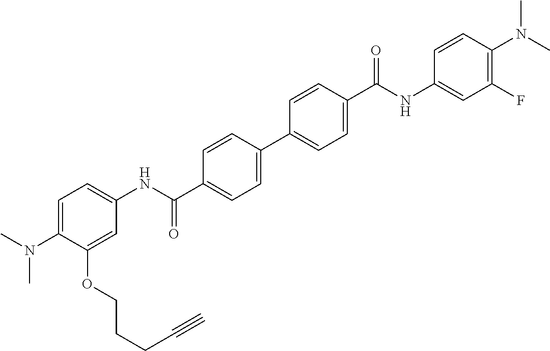 |
| 124 | 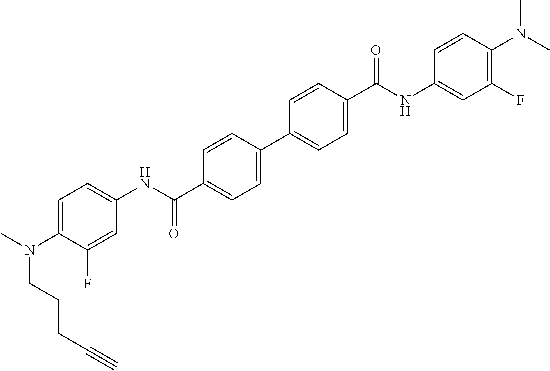 |
| 125 | 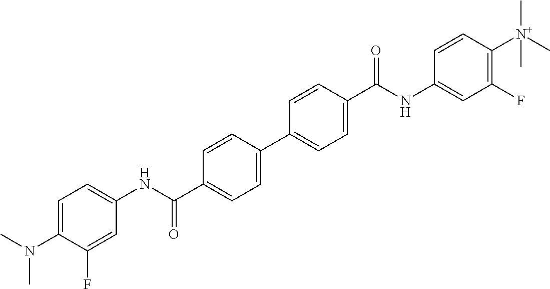 |
| 126 | 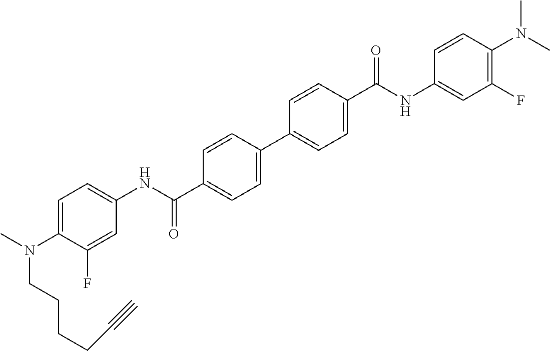 |
| 127 | 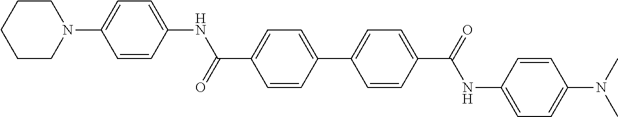 [SMILES: CN(C)c1ccc(NC(=O)c2ccc(-c3ccc(C(=O)Nc4ccc(N5CCCCC5)cc4)cc3)cc2)cc1] |
| 128 | 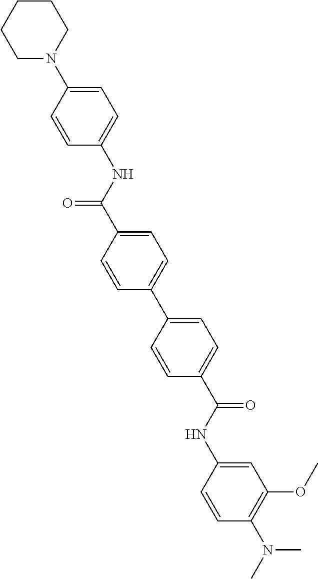 [SMILES: COc1cc(NC(=O)c2ccc(-c3ccc(C(=O)Nc4ccc(N5CCCCC5)cc4)cc3)cc2)ccc1N(C)C] |
| 129 | 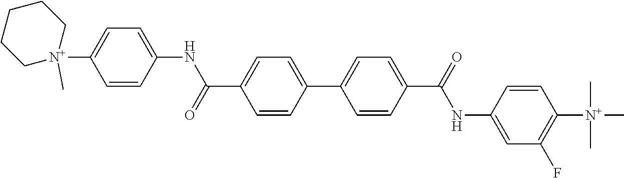 [SMILES: C[N+](C)(C)c1ccc(NC(=O)c2ccc(-c3ccc(C(=O)Nc4ccc([N+]5(C)CCCCC5)cc4)cc3)cc2)cc1F] |
| 130 | 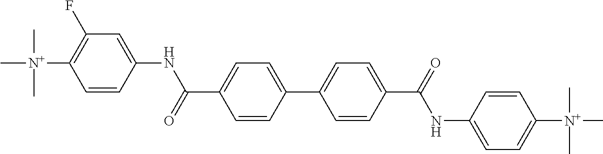 |
| 131 | 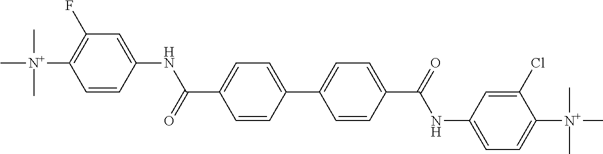 |
| 132 | 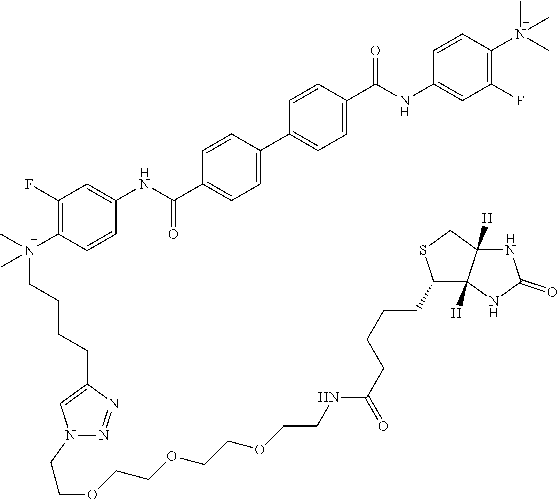 |
| 133 | 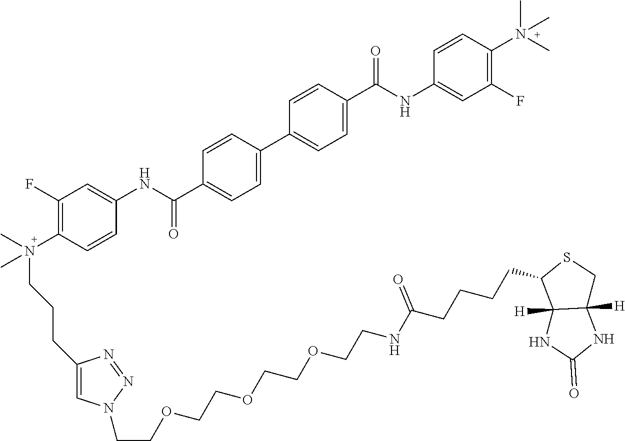 |
| 134 | 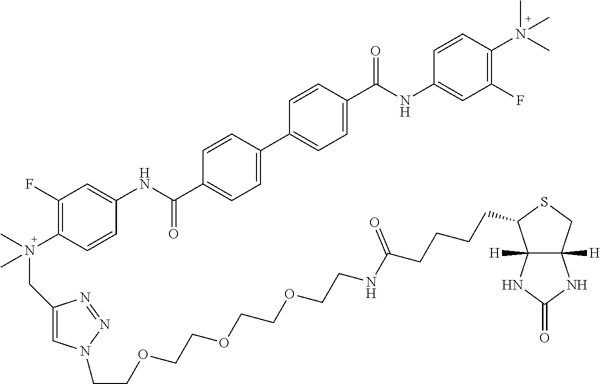 |
| 135 | 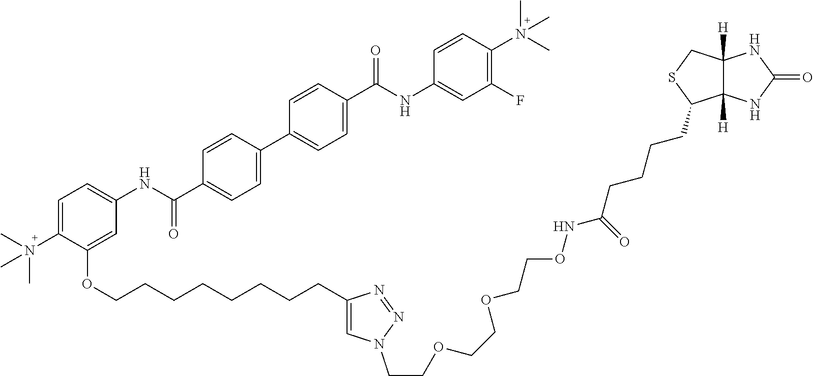 |
| 136 | 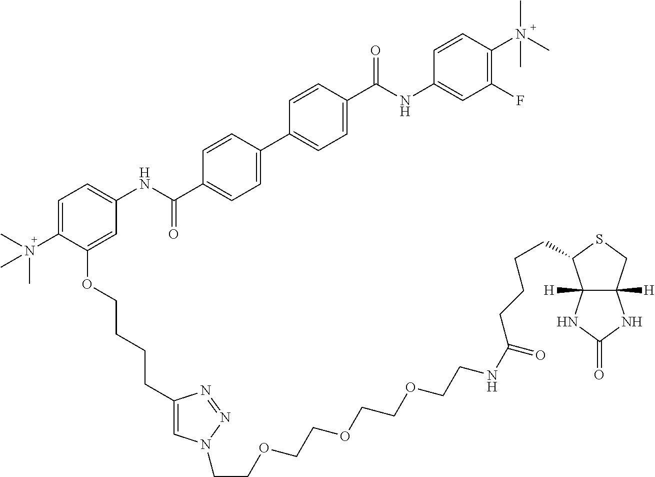 |
| 137 | 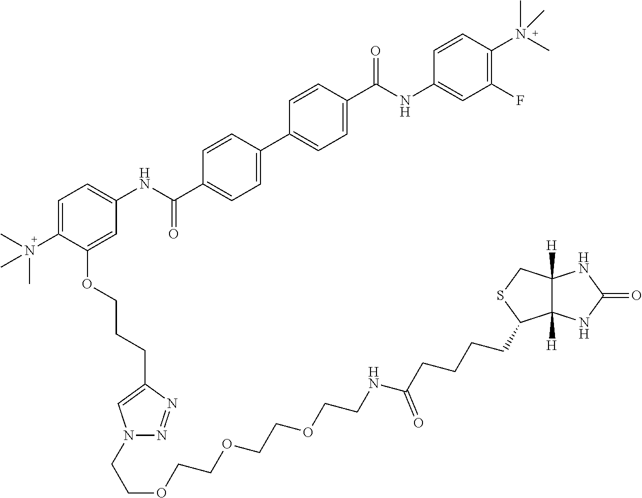 |
| 138 | 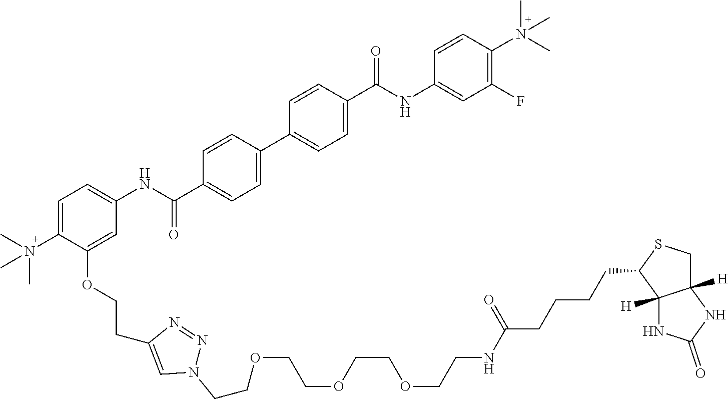 |
| 139 | 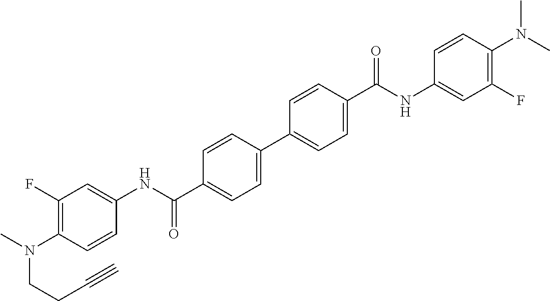 |
| 140 | 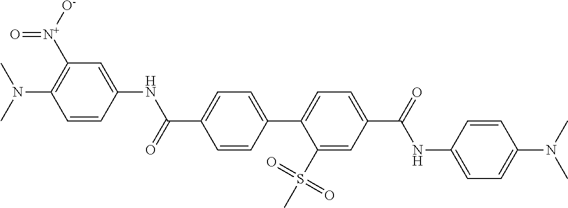 |
| 141 | 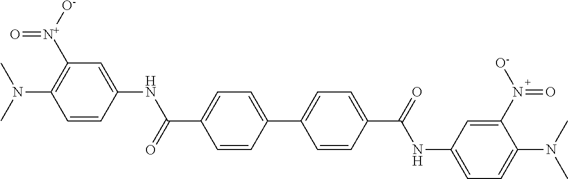 |
| 142 | 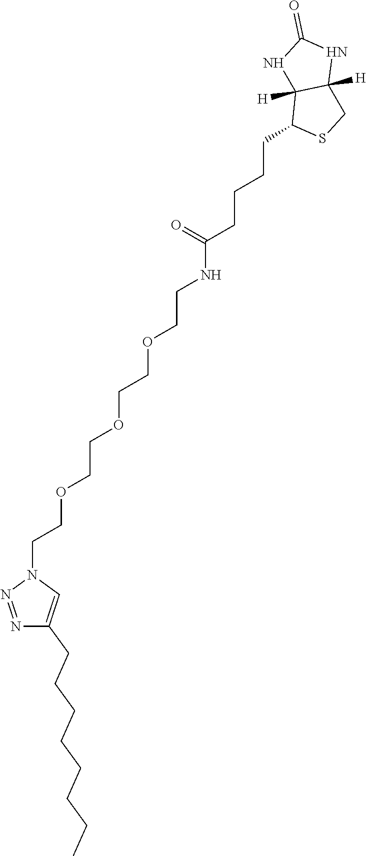 [SMILES: CCCCCCCCc1cn(CCOCCOCCOCCNC(=O)CCCC[C@H]2SC[C@H]3NC(=O)N[C@H]32)nn1] |
| 143 | 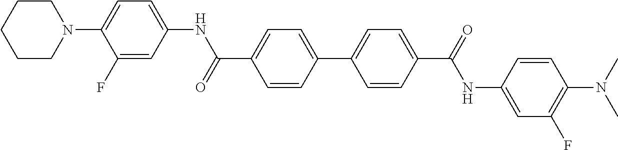 [SMILES: CN(C)c1ccc(NC(=O)c2ccc(-c3ccc(C(=O)Nc4ccc(N5CCCCC5)c(F)c4)cc3)cc2)cc1F] |
| 144 | 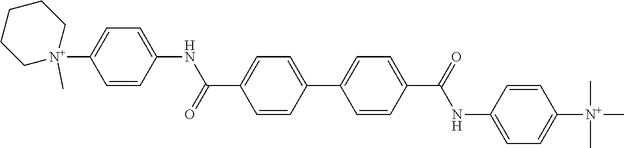 [SMILES: C[N+](C)(C)c1ccc(NC(=O)c2ccc(-c3ccc(C(=O)Nc4ccc([N+]5(C)CCCCC5)cc4)cc3)cc2)cc1] |
| 145 | 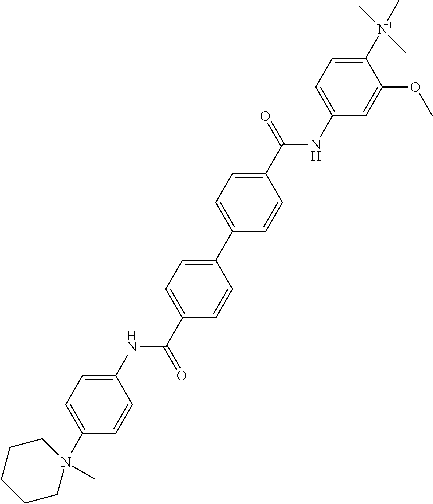 [SMILES: COc1cc(NC(=O)c2ccc(-c3ccc(C(=O)Nc4ccc([N+]5(C)CCCCC5)cc4)cc3)cc2)ccc1[N+](C)(C)C] |
| 146 | 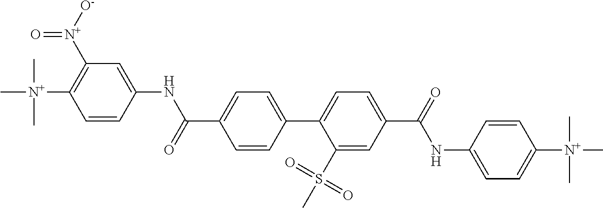 |
| 147 | 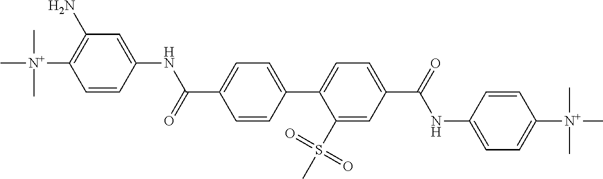 |
| 148 | 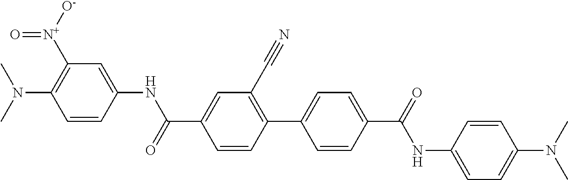 |
| 149 | 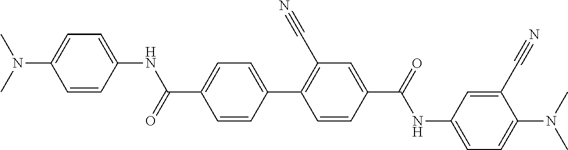 |
| 150 | 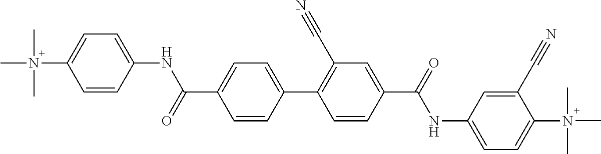 |
| 151 | 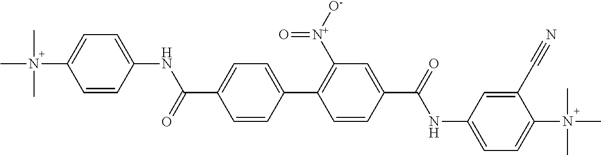 |
| 152 | 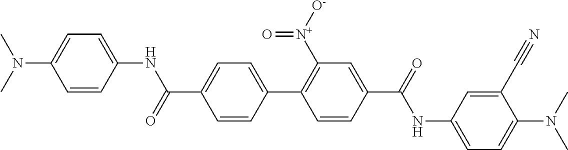 |
| 153 | 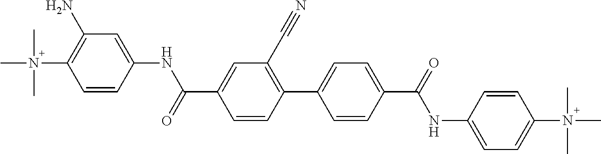 |
| 154 | 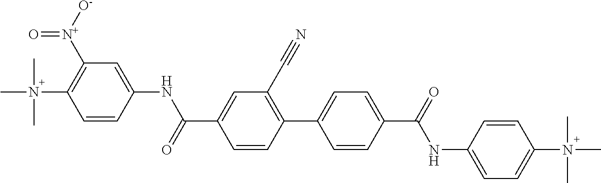 |
| 155 | 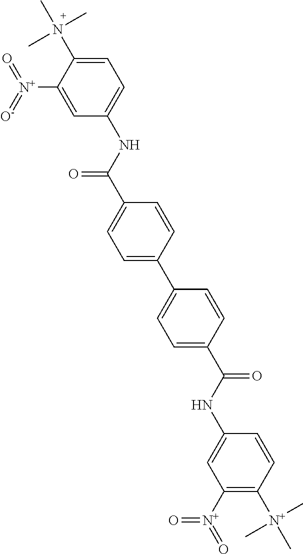 |
| 156 | 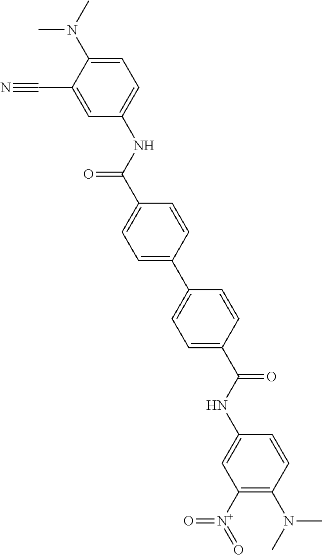 |
| 157 | 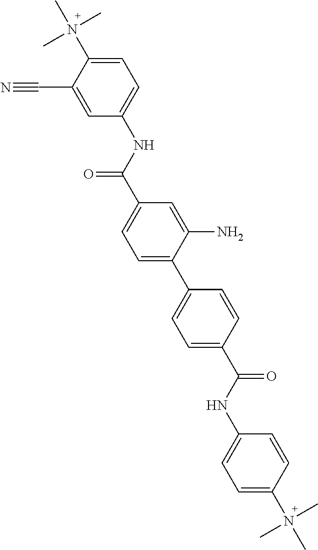 |

### Example 2 Biologic Studies

A. Microbiology Methods

i. Bacterial Strains

The Escherichia coli strains used in this study are listed in Table 1. Other strains used to assess the microbiological spectrum of activity are listed in Table 2.

B. Bacterial Culture and Minimum Inhibitory Concentration

Luria-Bertani (LB) broth (10 g of Bacto tryptone, 5 g of yeast extract, and 5 g of NaCl per liter; pH 7.0) was used for bacterial growth. The bacterial strains were inoculated overnight at 37° C. Cells were 1:100 subcultured into a fresh 30-ml volume of LB medium and grown at 37° C. to an optical density at 600 nm (OD600) of 0.3. When appropriate, the cells cultures were supplemented with 0.1% L-arabinose or 0.1 mM isopropyl β-d-1-thiogalactopyranoside (IPTG), to induce expression of the “Pore” protein. The cells were cultured up to OD600 of 1.0. The test compounds were dissolved in dimethylsulfoxide (DMSO) to make the final 10 mM stock solution. The minimal inhibitory concentrations (MIC) were determined using the 2-fold broth dilution method (1). The OD600 was measured using a Tecan Spark 10 M microplate reader.

C. Cytotoxicity Assay

Cytotoxicity of the compounds was evaluated using the CellTiter AQueous One Solution Cell Proliferation Assay (MTS; Promega) as previously described (2). HEK293 cells were incubated with a 2-fold serially diluted compounds for 24 h at 37° C. with 5% CO₂, followed by addition of 20 μL of CellTiter AQueous and further incubated for 4 h at 37° C. The absorbance at 490 nm was recorded using a Tecan Spark 10 M plate reader. All measurements were done in three replicas. The growth curves were then fitted to a sigmoidal equation to determine the cytotoxicity concentration CC₅₀.

D. Peritonitis Model of Infection

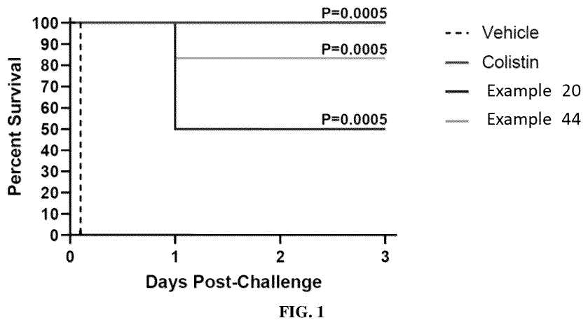

ICR mice were infected with NDM1 E. coli (ATCC BAA-2469) via IP injection at a targeted challenge of 7.6 log 10 CFU with 5% mucin. Mice were treated with test articles via IP injection at 30 minutes post challenge, or colistin sulfate via SC injection 30 min following challenge. Mice were monitored for survival through Day 3. Results are shown in FIG. 1.

TABLE 2
Escherichia coli strains used in this study

| | | Source |
|---|---|---|
| | | or |
| Strain | Relevant genotype or description | reference |
| GKCW101 | BW25113 attTn7::mini-Tn7T Km^{r} araC ParaBAD MCS | (1) |
| (WT) | | |
| GKCW102 | BW25113 attTn7::mini-Tn7T Km^{r} araC ParaBAD | (1) |
| (WT-Pore) | fhuAΔC/Δ4L | |
| GKCW103 | BW25113 ΔtolC ygiBC attTn7::mini-Tn7T Km^{r} araC | (1) |
| (ΔTolC) | ParaBAD MCS | |
| GKCW104 | BW25113 ΔtolC ygiBC attTn7::mini-Tn7T Km^{r} araC | (1) |
| (ΔTolC-Pore) | ParaBAD fhuAΔC/Δ4L | |

TABLE 3
Strains used in this study

| | | | Source or |
|---|---|---|---|
| Strain | Permeability | MDR | reference |
| Klebsiella pneumoniae ATCC13883 | WT | | ATCC |
| Klebsiella aerogenes ATCC13048 | WT | | ATCC |
| Klebsiella oxytoca ATCC700324 | WT | | ATCC |
| Klebsiella oxytoca ATCC700324 Pore | Permeable | | Dr. Zgurskaya |
| Enterobacter cloacae ATCC13047 | WT | | ATCC |
| Pseudomonas aeruginosa MP 2019 | WT | MDR | ATCC |
| Pseudomonas aeruginosa BAA 2108 | WT | MDR | ATCC |
| Pseudomonas aeruginosa PAO1 MCS | WT | | (3) |
| PAO1 Δ6-Pore | Permeable | | (3) |
| Pseudomonas aeruginosa 273 | WT | Clinical non | Dr. Callegan |
| | | MDR | |
| Pseudomonas aeruginosa 462 | WT | Clinical non | Dr. Callegan |
| | | MDR | |
| Pseudomonas aeruginosa 515 | WT | Clinical non | Dr. Callegan |
| | | MDR | |
| Acinetobacter baumannii AYE | WT | MDR | (4) |
| Acinetobacter baumannii AYE ΔB | Permeable | MDR | (4) |
| ΔIJK | | | |
| Acinetobacter baumannii AYE ΔB- | Permeable | MDR | (4) |
| Pore | | | |
| Acinetobacter baumannii Ab5075 | WT | MDR | (4) |
| Acinetobacter baumannii Ab5075 | Permeable | MDR | (4) |
| ΔIJK | | | |
| Acinetobacter baumannii ATCC17978 | WT | | ATCC |
| Acinetobacter baumannii Δ3-Pore | Permeable | | (5) |
| Burkholderia thailandensis E264 | WT | | (5) |
| Burkholderia thailandensis Δ2-Pore | WT | | (5) |
| Yersinia pestis KIM6+ | WT | | Dr. Rybenkov |
| Staphylococcus aureus | WT | non MDR | ATCC |
| (ATCC 25923) | | | |
| Staphylococcus aureus | WT | MDR | ATCC |
| ATCC 700787 | | | |

TABLE 4
MIC of compounds for E. coli BW25113

| | |
|---|---|
| Example | MIC, μM |

| # | <1 | 1-10 | 10-50 | >50 |
|---|---|---|---|---|
| 1 | | | | * |
| 2 | | | | * |
| 3 | | | | * |
| 4 | | | | * |
| 5 | | | | * |
| 6 | | | | * |
| 7 | | | | * |
| 8 | | | * | |
| 9 | | | | * |
| 10 | | | | * |
| 11 | | | | * |
| 12 | | | | * |
| 13 | | | * | |
| 14 | | | | * |
| 15 | | | | * |
| 16 | | | | * |
| 17 | * | | | |
| 18 | | | | * |
| 19 | | | | * |
| 20 | | * | | |
| 21 | | | | * |
| 22 | | | | * |
| 23 | | | * | |
| 24 | | | | * |
| 25 | | * | | |
| 26 | | | | * |
| 27 | | | | * |
| 28 | | | | * |
| 29 | | | * | |
| 30 | | * | | |
| 31 | | | | * |
| 32 | | | | * |
| 33 | | | | * |
| 34 | | | | * |
| 35 | | | * | |
| 36 | | | | * |
| 37 | | | | * |
| 38 | | | | * |
| 39 | | * | | |
| 40 | | | | * |
| 41 | | | | * |
| 42 | | | | * |
| 43 | | | | * |
| 44 | | * | | |
| 45 | | | | * |
| 46 | | | | * |
| 47 | | | * | |
| 48 | | | | * |
| 49 | | | | * |
| 50 | | | | * |
| 51 | | | | * |
| 52 | | * | | |
| 53 | | | | * |
| 54 | | | | * |
| 55 | | | * | |
| 56 | | * | | |
| 57 | | | | * |
| 58 | | * | | |
| 59 | | | | * |
| 60 | | | | * |
| 61 | | | | * |
| 62 | | | * | |
| 63 | | | | * |
| 64 | | | | * |
| 65 | | | | |
| 66 | | | * | |
| 67 | | | | * |
| 68 | | | | * |
| 69 | | | | * |
| 70 | | | | * |
| 71 | | | | * |
| 72 | | | | * |
| 73 | | | | * |
| 74 | | | | * |
| 75 | | | | * |
| 76 | | | | * |
| 77 | | * | | |
| 78 | | | | * |
| 79 | | | | * |
| 80 | | | | * |
| 81 | | | | * |
| 82 | | | | * |
| 83 | | | | * |
| 84 | | | * | |
| 85 | | | | * |
| 86 | | | * | |
| 87 | | * | | |
| 88 | | * | | |
| 89 | | | * | |
| 90 | | | | * |
| 91 | | | | * |
| 92 | | | | * |
| 93 | | | | * |
| 94 | | * | | |
| 95 | | | | * |
| 96 | | * | | |
| 97 | | | | * |
| 98 | | * | | |
| 99 | | | | * |
| 100 | | | | * |
| 101 | | | | * |
| 102 | | | | * |
| 103 | | * | | |
| 104 | | | | * |
| 105 | | | | |
| 106 | | | | * |
| 107 | | | | * |
| 108 | | | | * |
| 109 | | | | |
| 110 | | | | |
| 111 | | | | * |
| 112 | | | | * |
| 113 | | | | |
| 114 | | | | * |
| 115 | | | | * |
| 116 | | * | | |
| 117 | | | | * |
| 118 | | | * | |
| 119 | | | * | |
| 120 | | * | | |
| 121 | | | | * |
| 122 | | | | * |
| 123 | | | | * |
| 124 | | | | * |
| 125 | | | | * |
| 126 | | | | * |
| 127 | | * | | |
| 128 | | * | | |
| 129 | | * | | |
| 130 | | | | * |
| 131 | | | | * |
| 132 | | | | * |
| 133 | | | | * |
| 134 | | | | * |
| 135 | | | | * |
| 136 | | | | * |
| 137 | | | | * |
| 138 | | | | * |
| 139 | | | | * |
| 140 | | | | * |
| 141 | | | | * |
| 142 | | * | | |
| 143 | | * | | |
| 144 | | | | * |
| 145 | | | | * |
| 146 | | | | * |
| 147 | | | | * |
| 148 | | | | * |
| 149 | | | | * |
| 150 | | | | * |
| 151 | | | | |
| 152 | | | | |
| 153 | | * | | |
| 154 | | | | * |
| 155 | | | | |
| 156 | | | | * |
| 157 | | | | |

TABLE 5
MIC (μM) of select compounds for wild
type and multidrug resistant bacteria

| Strain | 20 | 17 | 44 |
|---|---|---|---|
| Klebsiella pneumoniae ATCC13883 | 12.5 | 3.1 | 25 |
| Klebsiella aerogenes ATCC13048 | 12.5 | 6.2 | 50 |
| Klebsiella oxytoca ATCC700324 | 25 | >100 | 100 |
| Enterobacter cloacae ATCC13047 | 12.5 | 50 | 25 |
| Pseudomonas aeruginosa MP 2019 | 12.5 | >100 | 25 |
| Pseudomonas aeruginosa BAA 2108 | >100 | >100 | 100 |
| Pseudomonas aeruginosa PAO1 | 25 | 6.2 | 25 |
| Pseudomonas aeruginosa 273 | 12.5 | 12.5 | 25 |
| Pseudomonas aeruginosa 462 | 12.5 | 12.5 | 50 |
| Pseudomonas aeruginosa 515 | 25 | >100 | 50 |
| Acinetobacter baumannii AYE | 25 | >100 | 25 |
| Acinetobacter baumannii Ab5075 | 25 | 12.5 | 25 |
| Acinetobacter baumannii ATCC17978 | 3.1 | 6.2 | 3.1 |
| Burkholderia thailandensis | >100 | >100 | >100 |
| Yersinia pestis KIM6+ | 1.6 | 100 | 1.6 |
| Staphylococcus aureus | 0.8 | 0.8 | 0.4 |
| ATCC 25923 | | | |
| Staphylococcus aureus | 1.6 | 0.4 | 0.2 |
| ATCC 700787 | | | |

TABLE 6
MIC (μM) of select compounds for permeabilized bacteria

| Strain | 20 | 17 | 44 |
|---|---|---|---|
| Klebsiella oxytoca ATCC700324 Pore | 25 | >100 | 50 |
| Pseudomonas aeruginosa PAO1 Δ6-Pore | 0.2 | 0.2 | 0.4 |
| Acinetobacter baumannii AYE ΔB ΔIJK | 0.2 | 0.2 | 0.1 |
| Acinetobacter baumannii Ab5075 ΔIJK | 25 | 6.2 | 12.5 |
| Acinetobacter baumannii ATCC17978 Δ3-Pore | 0.8 | 0.8 | 0.4 |
| Burkholderia thailandensis Δ2-Pore | 6.2 | 0.8 | 6.2 |

TABLE 7
Cytotoxicity of select compounds

| Example | CC₅₀, μM |
|---|---|
| 17 | >1000 |
| 20 | >1000 |
| 25 | 350 |
| 30 | 480 |
| 35 | 650 |
| 39 | 330 |
| 44 | 290 |
| 47 | 350 |

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference:
Bilici et al., Front. Chem. 9, 2021.
Chapkin et al., Proc. Natl. Acad. Sci., 115:9134-39, 2018.
Cui et al., Nano Lett., 16:6390-95, 2016.
Lange et al., Pharmaceutics, 13, 2021.
Li et al., Bioact. Mater. 6:794-809, 2021.
Liu et al., ACS Nano, 13:6813-23, 2019.
Mishra et al., Chem. Rev., 100:1973-2012, 2000.
Mustroph, Phys. Sci. Rev, https://doi.org/10.1515/psr-2020-0145, 2021.
Mustroph & Towns, ChemPhysChem, 19:1016-23, 2018.
Orlandi & Siebrand, J. Chem. Phys., 58:4513, 2003.
Qian et al., J. Phys. Chem. A, 124:9156-65, 2020.
Shi et al., J. Biomed. Opt., 21:050901, 2016.
Štacková et al., J. Org. Chem., 85:9776-90, 2020.
Weissleder, Nat. Biotechnol., 19(4):316-17, 2001.
Mishra et al., Chem. Rev., 100:1973-2012, 2000.
Zhao et al., ACS Infect. Dis., 4; 185-195, 2018.
Krishnamoorthy et al., Antimicrob. Agents Chemother., 60:7372-7381, 2016.
Krishnamoorthy et al., mBio, 8, 2017.
Leus et al., Mol. Microbiol., 114(6):1049-1065, 2020.
Wolloscheck et al., ACS Infect. Dis., 4:185-195, 2018.

1. A compound of the formula: 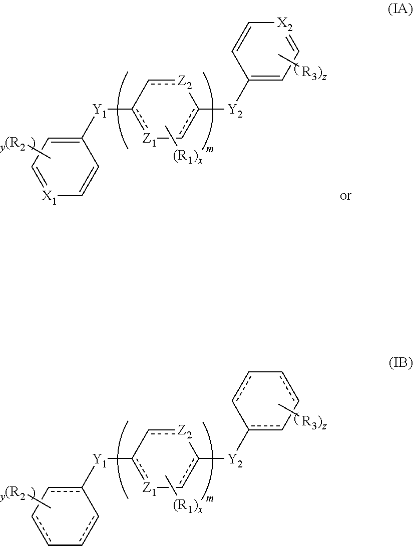
wherein:
m is 1 or 2;
X₁ and X₂ are each C, N, or ⁺NRₐ; wherein:
Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
R_{b} is hydrogen, alkyl_{(C≤6)}, or substituted alkyl_{(C≤6)}; and
a is 0, 1, or 2;
Each R₁ is amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, alkylsulfonyl_{(C≤8)}, substituted alkylsulfonyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x is 0, 1, 2, 3, or 4;
each Z₁ and Z₂ in each ring are each independently C, N, S, or O;
R₂ and R₃ are each independently amino, cyano, halo, hydroxy, nitro, aminosulfonyl, hydroxysulfonyl, alkyl_{(C≤12)}, substituted alkyl_{(C≤12)}, alkenyl_{(C≤12)}, substituted alkenyl_{(C≤12)}, alkynyl_{(C≤12)}, substituted alkynyl_{(C≤12)}, heterocycloalkyl_{(C≤8)}, substituted heterocycloalkyl_{(C≤8)}, alkoxy_{(C≤8)}, substituted alkoxy_{(C≤8)}, alkenyloxy_{(C≤8)}, substituted alkenyloxy_{(C≤8)}, alkynyloxy_{(C≤8)}, substituted alkynyloxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}; or
a group of the formula: ⁺N(R_{c})₃ wherein:
each R_{c} are each independently alkyl_{(C≤6)} or substituted alkyl_{(C≤6)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
or a pharmaceutically acceptable salt thereon.

2. The compound of claim 1 further defined as: 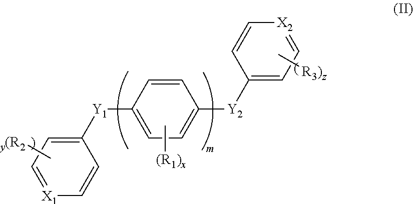
wherein:
m is 1 or 2;
X₁ and X₂ are each CH, N, or NRₐ; wherein:
Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
R_{b} is hydrogen, alkyl_{(C≤6)}, or substituted alkyl_{(C≤6)}; and
a is 0, 1, or 2;
Each R₁ is amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x is 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
or a pharmaceutically acceptable salt thereon.

3. The compound of claim 1 further defined as: 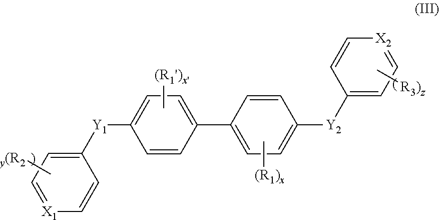
wherein:
X₁ and X₂ are each CH, N, or NRₐ; wherein:
Rₐ is alkyl_{(C≤6)} or substituted alkyl_{(C≤6)};
Y₁ and Y₂ are each C(O)O, C(O)NR_{b}, S(O)ₐ, or S(O)ₐNR_{b}; wherein:
R_{b} is hydrogen, alkyl_{(C≤6)}, or substituted alkyl_{(C≤6)}; and
a is 0, 1, or 2;
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:
—X₃—Y₃—R₄
wherein:
X₃ is a covalent bond, O, NR, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
or a pharmaceutically acceptable salt thereon.

4. The compound of claim 1 further defined as: 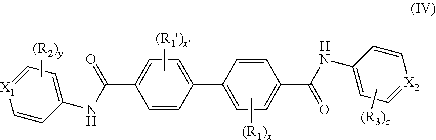
wherein:
X₁ and X₂ are each CH, N, or NRₐ; wherein:
Rₐ is alkyl_{(C≤6)} or substituted alkyl_{(C≤6)};
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
or a pharmaceutically acceptable salt thereon.

5. The compound of claim 1 further defined as: 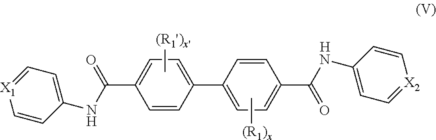
wherein:
X₁ and X₂ are each CH, N, or NRₐ; wherein:
Rₐ is alkyl_{(C≤6)} or substituted alkyl_{(C≤6)};
Each R₁ and R₁′ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)},
x and x′ are each independently 0, 1, 2, 3, or 4;
R₂ and R₃ are each independently amino, cyano, halo, hydroxy, nitro, aminosulfonyl, hydroxysulfonyl, alkyl_{(C≤8)}, substituted alkyl_{(C≤8)}, alkoxy_{(C≤8)}, substituted alkoxy_{(C≤8)}, acyl_{(C≤8)}, substituted acyl_{(C≤8)}, amido_{(C≤8)}, substituted amido_{(C≤8)}, alkylamino_{(C≤8)}, substituted alkylamino_{(C≤8)}, dialkylamino_{(C≤8)}, or substituted dialkylamino_{(C≤8)}, or a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}; and
y and z are each independently 1, 2, 3, 4, or 5
provided that if X₁ or X₂ is CH, then at least one or R₂ or R₃ is a group of the formula: —X₃—Y₃—R₄;
wherein if the compound contains one or more cationic groups; the compound further comprises one or more anionic groups to balance the charge of the cationic groups;
or a pharmaceutically acceptable salt thereon.

6.-11. (canceled)

12. The compound of claim 1, wherein y is 0, 1, or 2.

13.-18. (canceled)

19. The compound wherein R₂ is a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}.

20.-26. (canceled)

27. The compound of claim 19, wherein Y₃ is arenediyl_{(C≤12)} or substituted arenediyl_{(C≤12)}.

28.-29. (canceled)

30. The compound of claim 19, wherein Y₃ is heteroarenediyl_{(C≤12)} or substituted heteroarenediyl_{(C≤12)}.

31.-32. (canceled)

33. The compound of claim 19, wherein R₄ is alkyl_{(C≤12)} or substituted alkyl_{(C≤12)}.

34. The compound of claim 33, wherein R₄ is on a nitrogen atom of a heteroarenediyl group at Y₃.

35.-36. (canceled)

37. The compound of claim 19, wherein R₄ is ⁺N(R₅)₃.

38.-41. (canceled)

42. The compound of claim 1, wherein R₃ is alkyl_{(C≤8)} or substituted alkyl_{(C≤8)}.

43.-47. (canceled)

48. The compound of claim 1, wherein R₃ is a group of the formula:
—X₃—Y₃—R₄
wherein:
X₃ is a covalent bond, O, NR_{c}, S, alkanediyl_{(C≤6)}, or substituted alkanediyl_{(C≤6)};
Y₃ is arenediyl_{(C≤12)}, heteroarenediyl_{(C≤12)}, or a substituted version of either of these groups; and
R₄ is alkyl_{(C≤12)}, substituted alkyl_{(C≤12)}, or ⁺N(R₅)₃, wherein:
R₅ is hydrogen, alkyl_{(C≤8)}, or substituted alkyl_{(C≤8)}.

49.-55. (canceled)

56. The compound of claim 48, wherein Y₃ is arenediyl_{(C≤12)} or substituted arenediyl_{(C≤12)}.

57.-58. (canceled)

59. The compound of claim 48, wherein Y₃ is heteroarenediyl_{(C≤12)} or substituted heteroarenediyl_{(C≤12)}.

60.-61. (canceled)

62. The compound of claim 48, wherein R₄ is alkyl_{(C≤12)} or substituted alkyl_{(C≤12)}.

63.-69. (canceled)

70. The compound of claim 1, wherein the compound is further defined as:

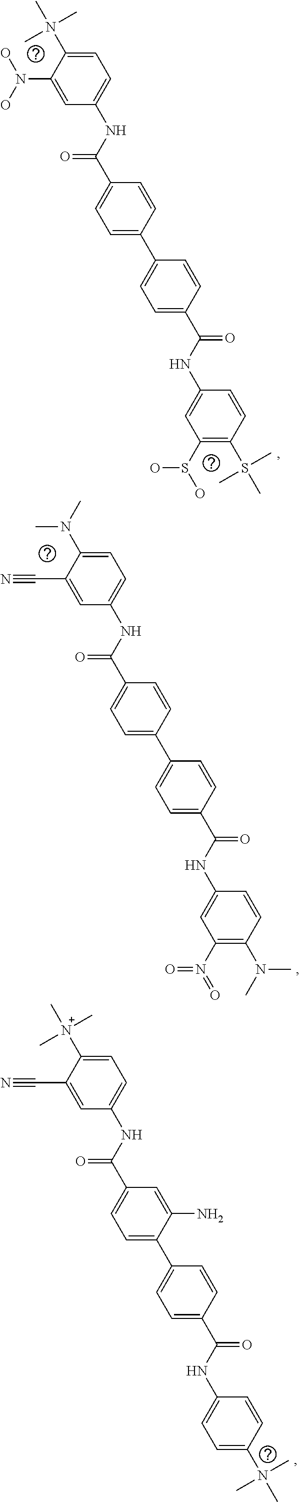

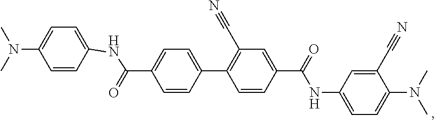

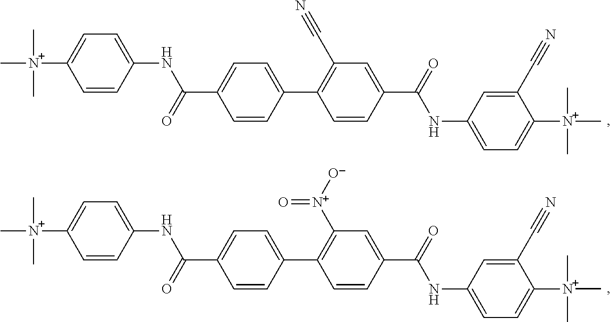

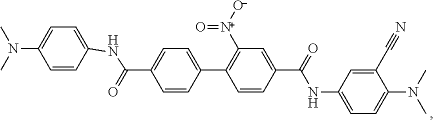

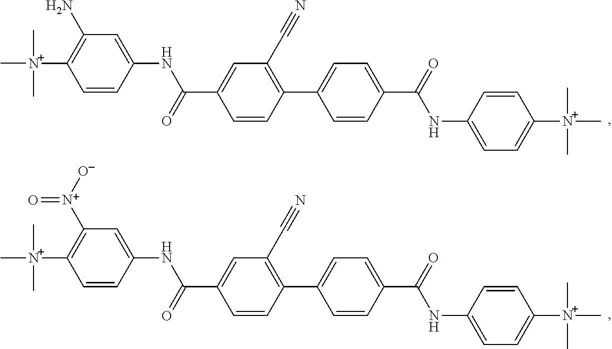

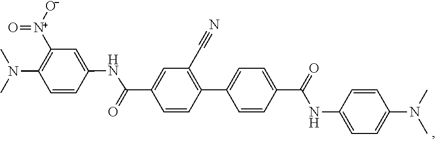

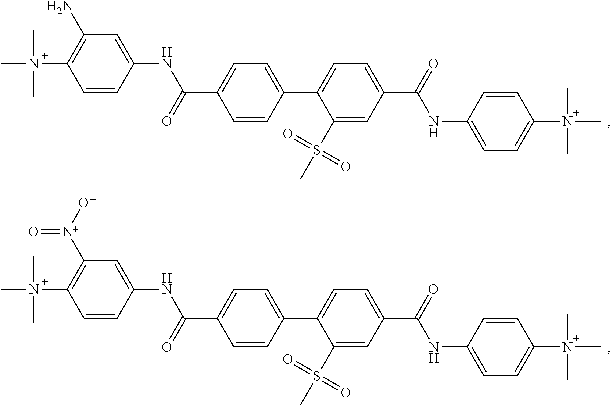

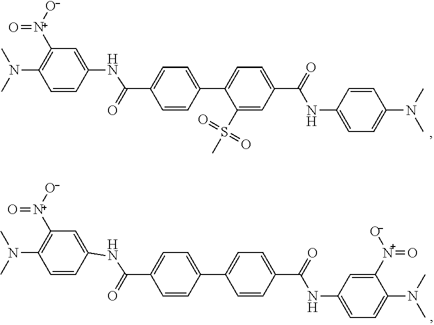

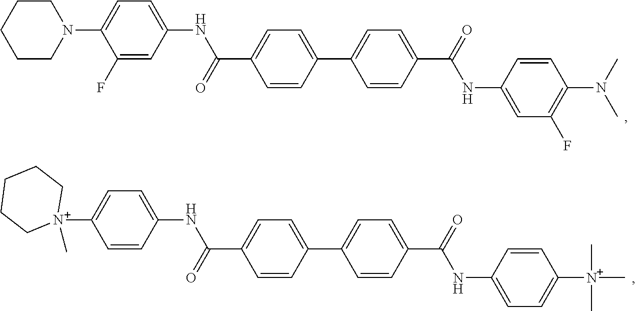 [SMILES: CN(C)c1ccc(NC(=O)c2ccc(-c3ccc(C(=O)Nc4ccc(N5CCCCC5)c(F)c4)cc3)cc2)cc1F ; C[N+](C)(C)c1ccc(NC(=O)c2ccc(-c3ccc(C(=O)Nc4ccc([N+]5(C)CCCCC5)cc4)cc3)cc2)cc1]

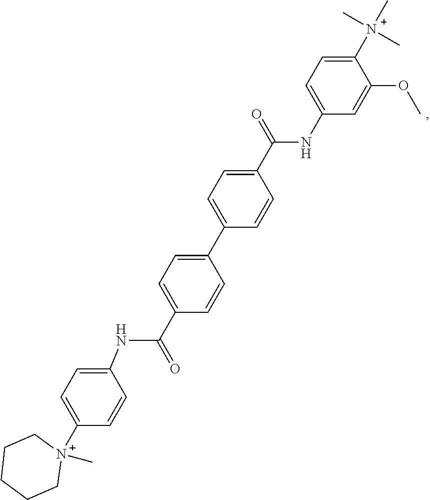 [SMILES: COc1cc(NC(=O)c2ccc(-c3ccc(C(=O)Nc4ccc([N+]5(C)CCCCC5)cc4)cc3)cc2)ccc1[N+](C)(C)C]

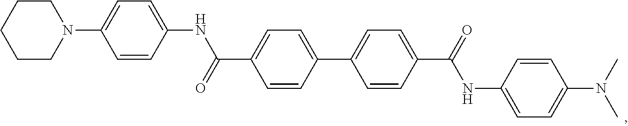 [SMILES: CN(C)c1ccc(NC(=O)c2ccc(-c3ccc(C(=O)Nc4ccc(N5CCCCC5)cc4)cc3)cc2)cc1]

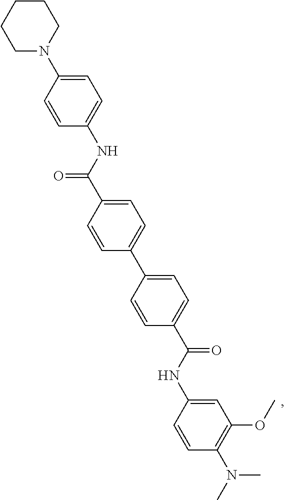 [SMILES: COc1cc(NC(=O)c2ccc(-c3ccc(C(=O)Nc4ccc(N5CCCCC5)cc4)cc3)cc2)ccc1N(C)C]

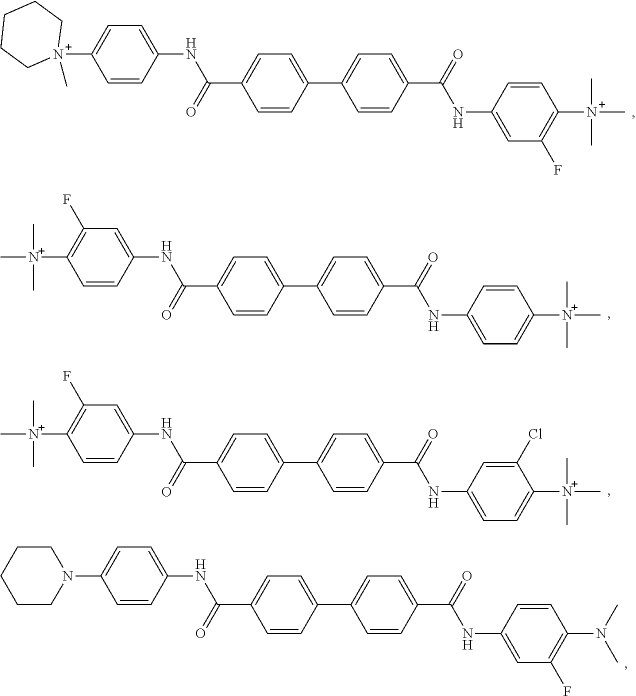

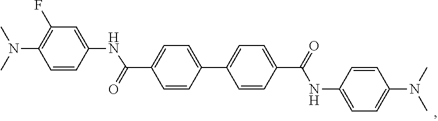

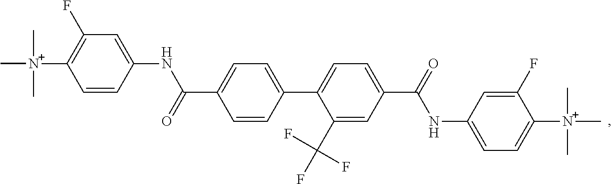

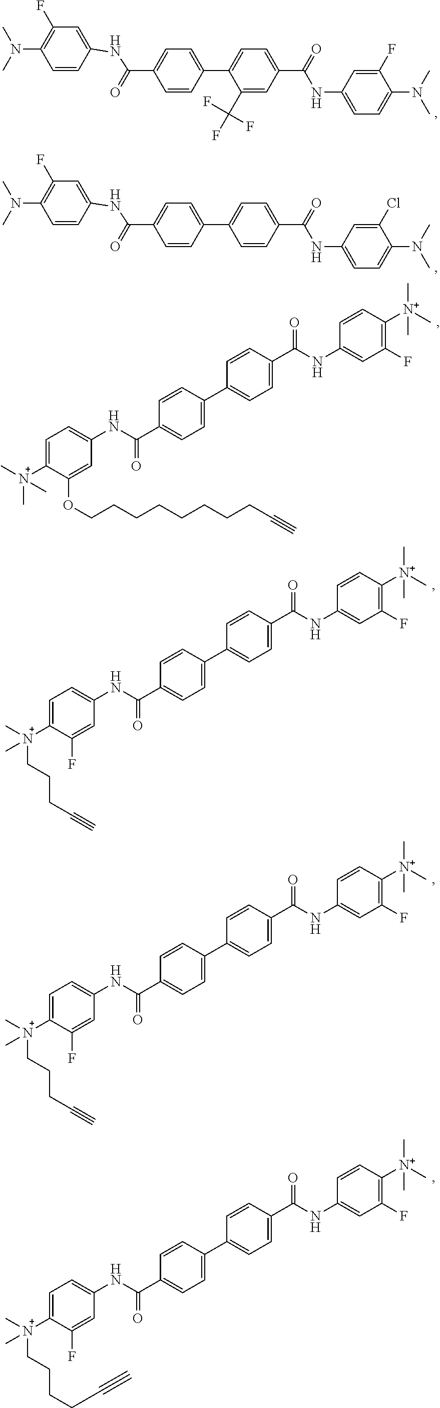

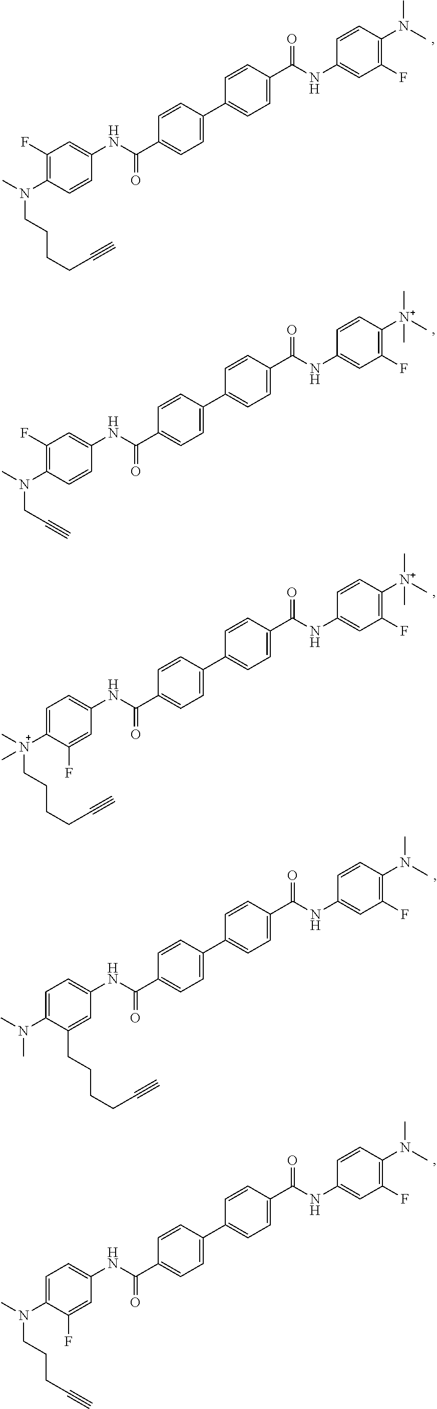

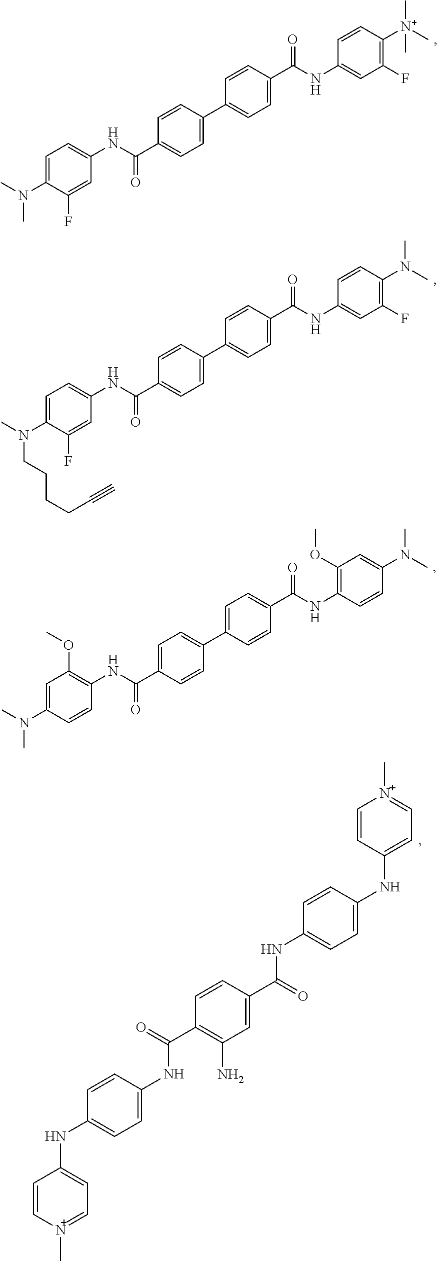

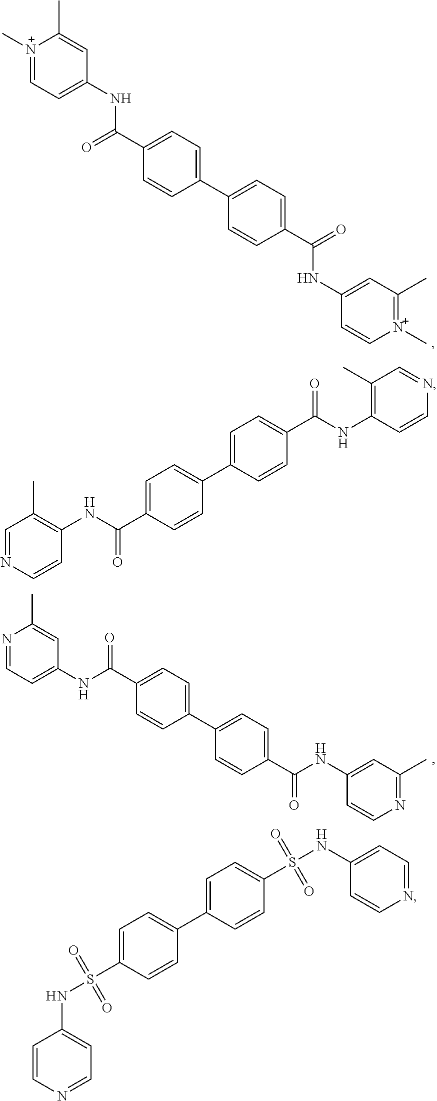

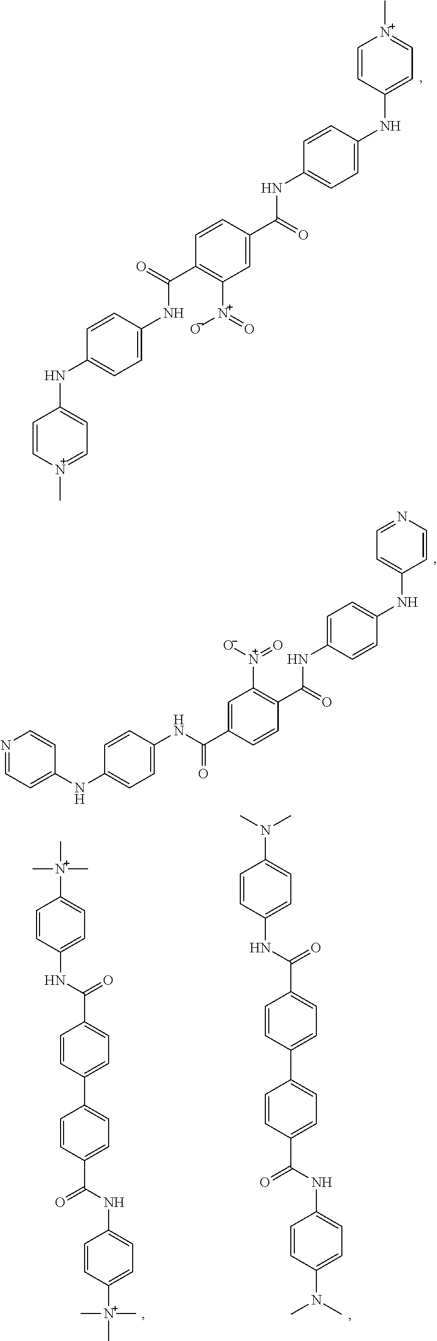

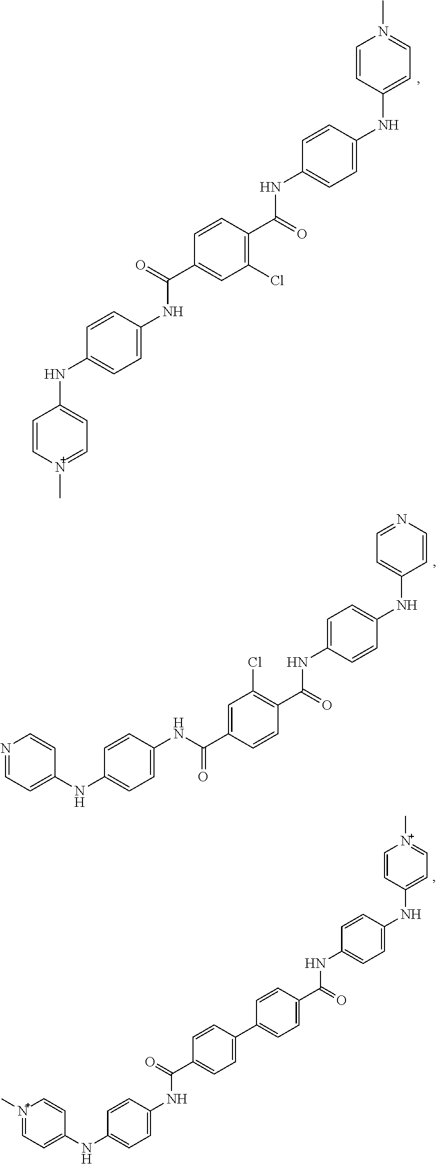

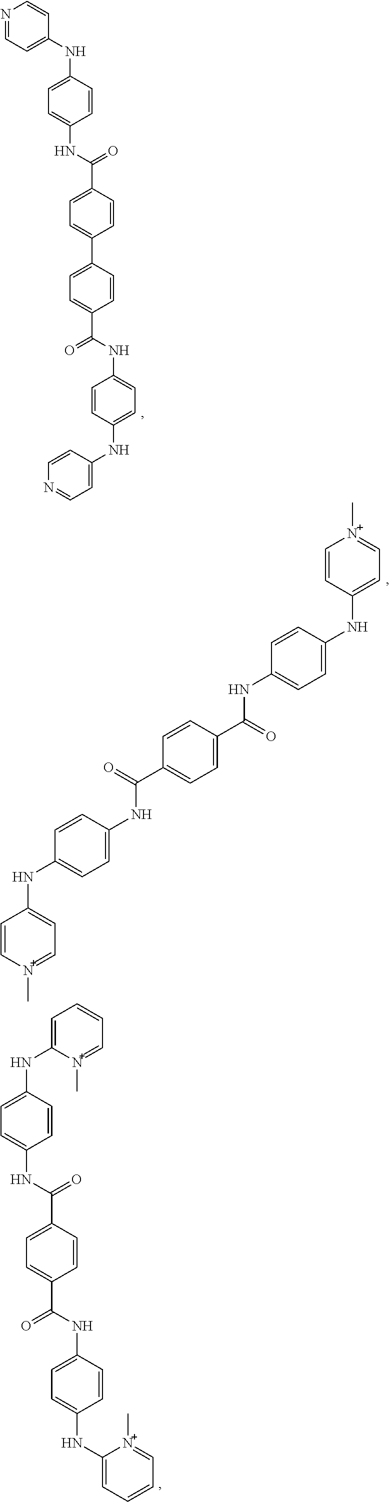 [SMILES: O=C(Nc1ccc(Nc2ccncc2)cc1)c1ccc(-c2ccc(C(=O)Nc3ccc(Nc4ccncc4)cc3)cc2)cc1 ; C[n+]1ccc(Nc2ccc(NC(=O)c3ccc(C(=O)Nc4ccc(Nc5cc[n+](C)cc5)cc4)cc3)cc2)cc1 ; C[n+]1ccccc1Nc1ccc(NC(=O)c2ccc(C(=O)Nc3ccc(Nc4cccc[n+]4C)cc3)cc2)cc1]

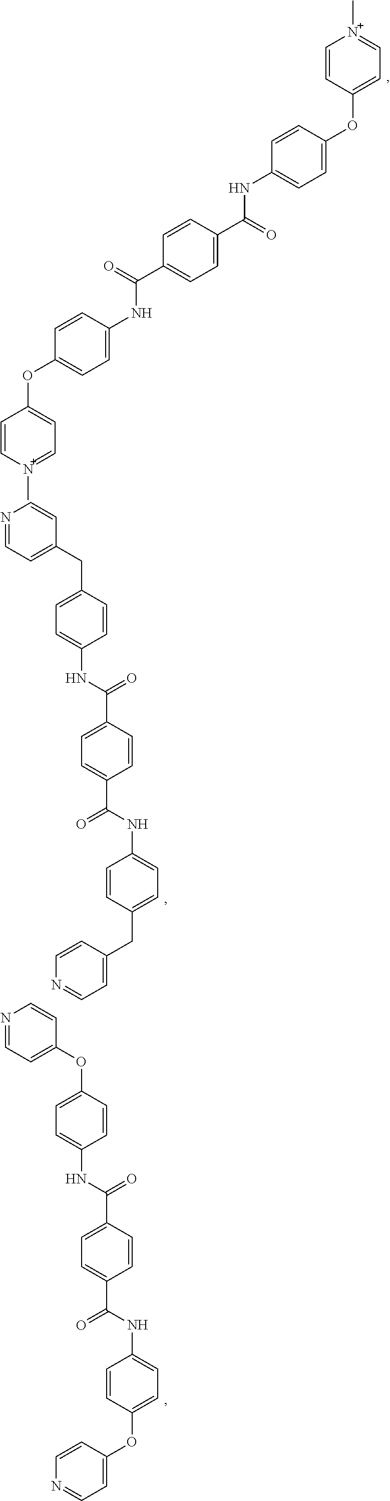

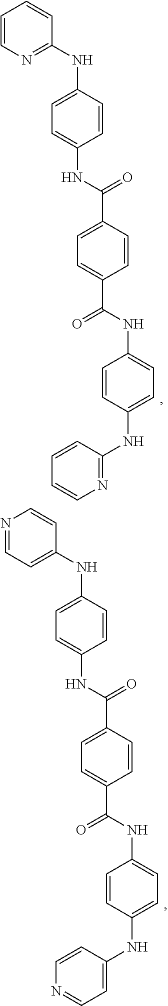

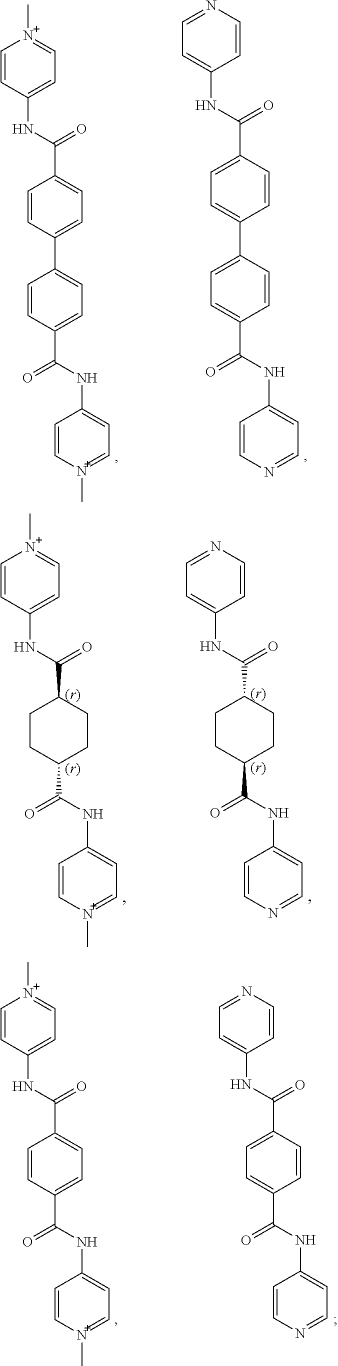 [SMILES: C[n+]1ccc(NC(=O)c2ccc(-c3ccc(C(=O)Nc4cc[n+](C)cc4)cc3)cc2)cc1 ; O=C(Nc1ccncc1)c1ccc(-c2ccc(C(=O)Nc3ccncc3)cc2)cc1 ; C[n+]1ccc(NC(=O)[C@H]2CC[C@H](C(=O)Nc3cc[n+](C)cc3)CC2)cc1 ; O=C(Nc1ccncc1)[C@H]1CC[C@H](C(=O)Nc2ccncc2)CC1 ; C[n+]1ccc(NC(=O)c2ccc(C(=O)Nc3cc[n+](C)cc3)cc2)cc1 ; O=C(Nc1ccncc1)c1ccc(C(=O)Nc2ccncc2)cc1]

71.-74. (canceled)

75. A pharmaceutical composition comprising:
(A) a compound of claim 1; and
(B) an excipient.

76.-77. (canceled)

78. A method of treating a disease or disorder in a patient comprising administering to the patient in need thereof a therapeutically effective amount of a compound of claim 1.

79.-90. (canceled)
